(19) United States
(12) Patent Application Publication
(10) Pub. No.: US 2021/0348120 A1
(43) Pub. Date: Nov. 11, 2021

(54) Polarised Three-Dimensional Cellular Aggregates

(71) Applicants: CAMBRIDGE ENTERPRISE LIMITED, Cambridgeshire (GB); ECOLE POLYTECHNIQUE FEDERALE DE LAUSANNE (EPFL), Lausanne (CH)

(72) Inventors: Alfonso Martinez Arias, Cambridgeshire (GB); Matthias Lutolf, St-Sulpice (CH); Cristina Pina, Cambridgeshire (GB); Giuliana Rossi, Lausanne (CH); Naomi Moris, Cambridgeshire (GB)

(21) Appl. No.: 17/277,192

(22) PCT Filed: Sep. 23, 2019
(86) PCT No.: PCT/GB2019/052668

§ 371 Date: Mar. 17, 2021

(30) Foreign Application Priority Data
Sep. 21, 2018 (GB) 1815438.5

(51) Int. Cl. C12N 5/00; C12N 5/074; C12N 5/073; C12N 5/077

(52) U.S. Cl. CPC C12N 5/0062; C12N 5/0696; C12N 5/0603; C12N 5/0657; C12N 2513/00; C12N 2506/45; C12N 2501/115; C12N 2501/165; C12N 2501/16; C12N 2506/02; C12N 2501/415

(57) ABSTRACT

Polarised three-dimensional cellular aggregates (or gastmloids) generated in vitro from one or more pluripotent stem cells are provided. Methods for obtaining polarised three-dimensional cellular aggregates and cells (e.g. progenitor cells and derivatives thereof) obtained from the polarised three-dimensional cellular aggregates are also provided.

### TECHNICAL FIELD

The present invention relates to polarised three-dimensional cellular aggregates generated in vitro from one or more pluripotent stem cells, methods for obtaining polarised three-dimensional cellular aggregates and cells obtained from the polarised three-dimensional cellular aggregates.

### BACKGROUND

The emergence of asymmetries within a mass of otherwise equivalent cells is the starting event in the development and patterning of all embryos, and results in the establishment of a coordinate system that cells use as a reference to generate the main axes of an organism. In chordate embryos the axial organisation acts as a reference for the process of gastrulation, a choreographed sequence of cell movements that transforms an epithelium into a three-layered structure endowed with a blueprint for the organism: a head at the anterior pole and, in vertebrates, the ectoderm, that will give rise to the nervous system on the dorsal side and the endoderm and the mesoderm on the ventral side. The process of gastrulation is driven by coordinated movements of groups of cells that interpret the global coordinate system of the embryo and give rise to the endoderm and the mesoderm.

Mouse embryonic stem cells (mESCs) are derived from the early mouse embryo and retain their ability to contribute to all embryonic tissues on reintroduction to a host embryo. Whereas mESCs have long been used as an in vitro model of embryogenesis when grown in the form of 3D embryoid bodies (EBs), these structures are typically formed from many hundreds to thousands of cells, and while they form many different cell types, their overall organisation is typically disordered with no reports of axial structures emerging (although polarisation in gene expression has been reported (Berge et al., 2008).

3D culturing protocols for aggregating mouse embryonic stem cells that display key features of early postimplantation mouse development have been reported (Baillie-Johnson et a., 2015, van den Brink et al., 2014, Turner et al., 2014 and Turner et al., 2017). These structures, known as gastruloids, exhibit an embryo-like spatiotemporal organization suggested to correspond to early post-implantation events in the embryo (Turner et al., 2014, Turner et al., 2017¹, Turner et al., 2017², and van den Brink et al., 2014). However, the lifespan of these gastruloids is limited with decreasing survival after 120 hours post-aggregation. In addition, the gastruloids described to date have not undergone organogenesis. These factors have hindered further development of gastruloids into developmentally and translationally relevant models of organ formation.

### DESCRIPTION

The invention provides polarised three-dimensional cellular aggregates (or gastruloids) generated in vitro from one or more pluripotent stem cells, methods for obtaining polarised three-dimensional cellular aggregates and cells (e.g. progenitor cells and derivatives thereof) obtained from the polarised three-dimensional cellular aggregates.

The methods of the invention enable the in vitro generation of polarised three-dimensional cellular aggregates (or gastruloids) that mimic the early post-implantation development of the embryo. When cultured according to the methods of the invention, gastruloids break radial symmetry, polarise their gene expression, and specify the major body axes. They further undergo a gastrulation-like process and axial elongation, and follow a temporal programme of gene expression that corresponds to post-occipital embryonic development, exemplified by the collinear expression of Hox genes along the developing anteroposterior axis. A key strength of this system is the ability to reproducibly and robustly generate large numbers of gastruloids under defined conditions that will enable demanding experimental approaches that would be very difficult or impossible to accomplish in the embryo alone.

Unlike existing methods of cell culture in which cells are kept as disorganised cellular aggregates (embryoid bodies) or artificial 2D cell layers, the polarised three-dimensional cellular aggregates of the invention promote cell-cell interactions in scaffold-free cell culture leading to the emergence of rare and mature cell types (e.g. primordial germ cells (PGCs), haematopoietic stem cells (HSCs) or haematopoietic progenitor cells, cardiac progenitor cells and neuromesodermal progenitors (NMps)) that form integrated tissue and organ structures.

Polarised three-dimensional cellular aggregates (or gastruloids) have a wide range of applications including: Antibody validation (the spatial localisation of a signal allows tests for specificity and background:noise validation for high-throughput early antibody screening); disease modelling and knockout (e.g. gene-editing) modelling (patient-specific or disease-relevant cell lines may be used to generate polarised three-dimensional cellular aggregates to model disease situations); providing a powerful in vitro research tool to gain insights into mechanistic events during post-implantation development including events that co-ordinate organ development with the potential to reduce, refine or replace embryonic material in research (the 3Rs); generation of cell types for in vitro toxicology studies; generation of cell types and tissue primordia for drug screening; induced pluripotent stem cell validation (since polarised three-dimensional cellular aggregates generate 3 germ layers with embryo-like organization, the aggregates may be used as a ‘read out’ assay of iPSC developmental potential, effectively bypassing the need for expensive and ethically-difficult mouse teratoma assays); generation of functional cell types, organs and tissues for regenerative medicine; generation of functional cell types as disease models for use in personalized medicine studies and drug discovery; and non-genetic prenatal diagnostics/testing (with the ability to start from one/few cells, polarised three-dimensional cellular aggregates may be used as a functional assay of developmental potential from blastomere cells of early IVF-embryos)

The polarised three-dimensional cellular aggregates are, like embryos, dynamic entities. These entities have emergent, embryo-like characteristics, in that over time they exhibit temporal sequences of the different combination of markers, gene expression patterns and morphological changes described herein.

The invention provides a polarised three-dimensional cellular aggregate generated in vitro from one or more pluripotent stem cells, wherein:
(a) the polarised three-dimensional cellular aggregate comprises
   i. cells comprising one or more markers characteristic of endodermal cells or derivatives thereof,
   ii. cells comprising one or more markers characteristic of mesodermal cells or derivatives thereof, and
   iii. cells comprising one or more markers characteristic of ectodermal cells or derivatives thereof; and
(b) the polarised three-dimensional cellular aggregate is polarised along the anterior-posterior, dorsal-ventral and medio-lateral axes, and wherein
   i. the anterior-posterior axis is defined by at least an anterior region of cells and a posterior region of cells, wherein the cells of the anterior region express a higher or lower level of one or more genes than the cells of the posterior region,
   ii. the dorsal-ventral axis is defined by at least a dorsal region of cells and a ventral region of cells, wherein the cells of the dorsal region express a higher or lower level of one or more genes than the cells of the ventral region, and
   iii. the medio-lateral axis is defined by at least a medial region of cells and two lateral regions of cells, wherein the cells of the medial region express a higher or lower level of one or more genes than the cells of the lateral regions.

The invention provides a polarised three-dimensional cellular aggregate generated in vitro from one or more pluripotent stem cells, wherein:
(a) the polarised three-dimensional cellular aggregate comprises cells comprising one or more markers characteristic of primordial germ cells or derivatives thereof; and
(b) the polarised three-dimensional cellular aggregate is polarised along the anterior-posterior the anterior-posterior axis, wherein the anterior-posterior axis is defined by at least an anterior region of cells and a posterior region of cells, and wherein the cells of the anterior region express a higher or lower level of one or more genes than the cells of the posterior region.

The polarised three-dimensional cellular aggregate may be polarised along the dorsal-ventral axis, wherein the dorsal-ventral axis is defined by at least a dorsal region of cells and a ventral region of cells, wherein the cells of the dorsal region express a higher or lower level of one or more genes than the cells of the ventral region.

The polarised three-dimensional cellular aggregate may be polarised along the medio-lateral, wherein the medio-lateral axis is defined by at least a medial region of cells and two lateral regions of cells, wherein the cells of the medial region express a higher or lower level of one or more genes than the cells of the lateral regions.

The invention provides a polarised three-dimensional cellular aggregate generated in vitro from one or more pluripotent stem cells, wherein:
(a) the polarised three-dimensional cellular aggregate comprises cells comprising one or more markers characteristic of primordial germ cells or derivatives thereof; and
(b) the polarised three-dimensional cellular aggregate is polarised along the anterior-posterior, dorsal-ventral and medio-lateral axes, and wherein
   i. the anterior-posterior axis is defined by at least an anterior region of cells and a posterior region of cells, wherein the cells of the anterior region express a higher or lower level of one or more genes than the cells of the posterior region,
   ii. the dorsal-ventral axis is defined by at least a dorsal region of cells and a ventral region of cells, wherein the cells of the dorsal region express a higher or lower level of one or more genes than the cells of the ventral region, and
   iii. the medio-lateral axis is defined by at least a medial region of cells and two lateral regions of cells, wherein the cells of the medial region express a higher or lower level of one or more genes than the cells of the lateral regions.

The invention provides a polarised three-dimensional cellular aggregate generated in vitro from one or more pluripotent stem cells, wherein:
(a) the polarised three-dimensional cellular aggregate comprises
   i. cells comprising one or more markers characteristic of endodermal cells or derivatives thereof,
   ii. cells comprising one or more markers characteristic of mesodermal cells or derivatives thereof,
   iii. cells comprising one or more markers characteristic of ectodermal cells or derivatives thereof, and
   iv. one or more markers characteristic of primordial germ cells or derivatives thereof; and
(b) the polarised three-dimensional cellular aggregate is polarised along the anterior-posterior, dorsal-ventral and medio-lateral axes, and wherein
   i. the anterior-posterior axis is defined by at least an anterior region of cells and a posterior region of cells, wherein the cells of the anterior region express a higher or lower level of one or more genes than the cells of the posterior region,
   ii. the dorsal-ventral axis is defined by at least a dorsal region of cells and a ventral region of cells, wherein the cells of the dorsal region express a higher or lower level of one or more genes than the cells of the ventral region, and
   iii. the medio-lateral axis is defined by at least a medial region of cells and two lateral regions of cells, wherein the cells of the medial region express a higher or lower level of one or more genes than the cells of the lateral regions.

The one or more markers may be gDNA, RNA, polypeptide or other molecules. Preferably, the one or more markers are genes the expression of which is characteristic of the specified cell type.

The one or more markers characteristic of primordial germ cells may be one or more genes the expression of which is characteristic of primordial germ cells. The one or more markers characteristic of primordial germ cells may be one or more genes the expression of which is characteristic of primordial germ cells, optionally wherein the one or more genes are selected from Prdm1, Prdm14, Dazl, Tfap2c, Nanos3. The one or more markers characteristic of primordial germ cells may be one or more markers characteristic of primordial germ cell derivatives.

The cells of the anterior region may express a lower level of one or more genes than the cells of the posterior region, and wherein the one or more genes are selected from Bra, Cdx1, Cdx2, Cdx4, Wnt3a, Cyp26a1, Fgf8, Wnt5a Tbx6, Msgn, Hes3, Chrd, Greb1, Rspo, Notum, Sall3, Sp5, Sp8 and Fgf4.

The cells of the anterior region may express a higher level of one or more genes than the cells of the posterior region, and wherein the one or more genes are selected from Gata6, Raldh2, Otx2, Pax3, Tbx1, Uncx4.1, Pax1, Six1, Meis1, Crabp1, Foxc2, Eya1, Flk1 and Lmo4. Flk1 may be expressed asymmetrically in the anterior region.

The cells of the anterior region may express a lower level of Bra than the cells of the posterior region, and wherein the cells of the anterior region express a higher level of Gata6 than the cells of the posterior region.

The cells of the anterior region may express a lower level of Bra, Cdx2, Wnt3a, Wnt5a Tbx6, Msgn, Hes3, Chrd, Greb1, Rspo, Notum, Sall3, Sp5, Sp8 and/or Fgf4 than the cells of the posterior region, and wherein the cells of the anterior region express a higher level of Gata6 and or Otx2 than the cells of the posterior region.

The cells of the anterior region may express a lower level of Bra, Cdx2, Wnt3a, Wnt5a Tbx6, Msgn, Hes3, Chrd, Greb1, Rspo, Notum, Sall3, Sp5, Sp8 and/or Fgf4 than the cells of the posterior region, and wherein the cells of the anterior region express a higher level of Tbx1 than the cells of the posterior region.

The cells of the anterior region express a lower level of Bra, Cdx2, Wnt3a, Wnt5a Tbx6, Msgn, Hes3, Chrd, Greb1, Rspo, Notum, Sall3, Sp5, Sp8 and/or Fgf4 than the cells of the posterior region, and wherein the cells of the anterior region express a higher level of Six1 than the cells of the posterior region.

The cells of the anterior region may express a lower level of Bra than the cells of the posterior region, and wherein the cells of the anterior region express a higher level of Gata6, Raldh2, Pax3, Tbx1, Uncx4.1, Pax1, Six1, Meis1, Crabp1, Foxc2, Eya1 and/or Lmo4 than the cells of the posterior region.

The cells of the anterior region may express a lower level of Wnt3a than the cells of the posterior region, and wherein the cells of the anterior region express a higher level of Gata6, Raldh2, Pax3, Tbx1, Uncx4.1, Pax1, Six1, Meis1, Crabp1, Foxc2, Eya1 and/or Lmo4 than the cells of the posterior region.

The cells of the anterior region may express a lower level of Tbx6 than the cells of the posterior region, and wherein the cells of the anterior region express a higher level of Gata6, Raldh2, Pax3, Tbx1, Uncx4.1, Pax1, Six1, Meis1, Crabp1, Foxc2, Eya1 and/or Lmo4 than the cells of the posterior region.

The anterior-posterior axis may be further defined by a central region of cells between the anterior region of cells and the posterior region of cells, wherein the cells of the central region express a higher or lower level of one or more genes than the cells of the anterior or posterior regions. The cells of the central region may express a higher level of one or more genes than the cells of the anterior or posterior regions, and wherein the one or more genes are selected from Cer1, Sox1, Sox2, Lnfg, Jag2, Lefty1, Utf1, Tbx3, Ripply2, Mesp1, and Mesp2.

The polarised three-dimensional cellular aggregate may exhibit spatial collinearity of Hox gene expression along the anterior-posterior axis. The polarised three-dimensional cellular aggregate may exhibit spatial and temporal collinearity of Hox gene expression along the anterior-posterior axis. The spatial collinearity of Hox gene expression along the anterior-posterior axis may comprise the sequential and ordered expression along this axis of Hox 1-13 from each of the a, b, c and d clusters. The spatial collinearity of Hox gene expression along the anterior-posterior axis may comprise the temporally sequential and ordered expression along this axis of Hox 1-13 from each of the a, b, c and d clusters.

The anterior region may consist of at least 2%, at least 5%, at least 10%, at least 15%, at least 20%, at least 25%, at least 30%, at least 35%, at least 40%, at least 45% or 50% of the polarised three-dimensional cellular aggregate. Preferably, the anterior region consists of at least 5% of the polarised three-dimensional cellular aggregate

The posterior region may consist of at least 2%, at least 5%, at least 10%, at least 15%, at least 20%, at least 25%, at least 30%, at least 35%, at least 40%, at least 45% or 50% of the polarised three-dimensional cellular aggregate. Preferably, the posterior region consists of at least 5% of the polarised three-dimensional cellular aggregate.

The central region may consist of at least 2%, at least 5%, at least 10%, at least 15%, at least 20%, at least 25%, at least 30%, at least 35%, at least 40%, at least 45% or 50% of the polarised three-dimensional cellular aggregate. Preferably, the central region consists of at least 5% of the polarised three-dimensional cellular aggregate.

The polarised three-dimensional cellular aggregate may comprise two or more of:
a. a region of cells expressing at least Bra,
b. a region of cells expressing at least Tbx6,
c. a region of cells expressing at least Meox1,
d. a region of cells expressing at least Mesp2,
e. a region of cells expressing at least Tcf15;
f. a region of cells expressing at least Gata6; and
g. a region of cells expressing at least Bmp2;
wherein (a)-(g) are arranged from posterior to anterior in the polarised three-dimensional cellular aggregate.

The cells of the dorsal region may express a lower level of one or more genes than the cells of the ventral region, and wherein the one or more genes are selected from Shh, Krt18, Pgg, Nedd9, Nodal, Lefty1, 2, Tbx6, Msgn FoxA2, and Kdr. The cells of the dorsal region may express a higher level of one or more genes than the cells of the ventral region, and wherein the one or more genes are selected from Sox2, Lnfg, Irx3, Sox1, and Pax7. The cells of the dorsal region may express a lower level of Shh than the cells of the ventral region, and wherein the cells of the dorsal region express a higher level of Sox2 than the cells of the ventral region.

The dorsal region may consist of at least 2%, at least 5%, at least 10%, at least 15%, at least 20%, at least 25%, at least 30%, at least 35%, at least 40%, at least 45% or 50% of the polarised three-dimensional cellular aggregate. Preferably, the dorsal region consists of at least 5% of the polarised three-dimensional cellular aggregate

The ventral region may consist of at least 2%, at least 5%, at least 10%, at least 15%, at least 20%, at least 25%, at least 30%, at least 35%, at least 40%, at least 45% or 50% of the polarised three-dimensional cellular aggregate. Preferably, the ventral region consists of at least 5% of the polarised three-dimensional cellular aggregate.

The cells of the medial region may express a lower level of one or more genes than the cells of the lateral regions, and wherein the one or more genes are selected from Osr1, Pecam, Meox1, Kdr, Meox1, Bmp4, Tbx6, Pax2, Lefty1, and Pitx2.

The cells of the medial region may express a higher level of one or more genes than the cells of the lateral regions, and wherein the one or more genes are selected from Sox2, Lfng, FoxA2, and Noto1.

The cells of the medial region may express a lower level of Meox1 and/or Pax2 than the cells of the lateral regions, and wherein the cells of the medial region express a higher level of Sox2 than the cells of the lateral regions. The cells of the medial region may express a lower level of Meox1 and/or Pax2 than the cells of the lateral regions, and wherein the cells of the medial region express a higher level of Sox1 than the cells of the lateral regions.

The medial region may consist of at least 2%, at least 5%, at least 10%, at least 15%, at least 20%, at least 25%, at least 30%, at least 35%, at least 40%, at least 45% or 50% of the polarised three-dimensional cellular aggregate. Preferably, the medial region consists of at least 5% of the polarised three-dimensional cellular aggregate

The lateral regions may consist of at least 2%, at least 5%, at least 10%, at least 15%, at least 20%, at least 25%, at least 30%, at least 35%, at least 40%, at least 45% or 50% of the polarised three-dimensional cellular aggregate. Preferably, the lateral regions consist of at least 5% of the polarised three-dimensional cellular aggregate.

The invention provides a polarised three-dimensional cellular aggregate generated in vitro from one or more pluripotent stem cells, wherein:
(a) the polarised three-dimensional cellular aggregate comprises
   i. cells comprising one or more markers characteristic of endodermal cells or derivatives thereof,
   ii. cells comprising one or more markers characteristic of mesodermal cells or derivatives thereof, and
   iii. cells comprising one or more markers characteristic of ectodermal cells or derivatives thereof; and
(b) the polarised three-dimensional cellular aggregate is polarised along the anterior-posterior, dorsal-ventral and medio-lateral axes, and wherein
   i. the anterior-posterior axis is defined by at least an anterior region of cells and a posterior region of cells, wherein the cells of the posterior region express a higher level of Bra, Wnt3a and/or Cyp26a1 than the cells of the anterior region,
   ii. the dorsal-ventral axis is defined by at least a dorsal region of cells and a ventral region of cells, wherein the cells of the ventral region express a higher level of Shh than the cells of the dorsal region, and
   iii. the medio-lateral axis is defined by at least a medial region of cells and two lateral regions of cells, wherein the cells of the lateral regions express a higher level of Meox1 and/or Pax2 than the cells of the medial region.

The one or more markers characteristic of endodermal cells or derivatives thereof may be one or more genes the expression of which is characteristic of endodermal cells or derivatives thereof. The one or more genes the expression of which is characteristic of endodermal cells or derivatives thereof may be selected from Gsc, Cdx2, Nedd9, Pyy, Shh, Sores, Cer1, Sox17 and FoxA1, FoxA2. The one or more genes the expression of which is characteristic of endodermal cells or derivatives thereof may be Sox17, Gsc and Cdx2.

The one or more genes the expression of which is characteristic of endodermal cells or derivatives thereof may be Shh and Sorcs2.

The one or more markers characteristic of derivatives of endodermal cells or derivatives thereof may be one or more genes the expression of which is characteristic of gut cells, optionally wherein the gut cells are foregut cells, midgut and/or hindgut cells. The one or more markers characteristic of derivatives of endodermal cells or derivatives thereof may be one or more genes the expression of which is characteristic of oesophagus, lung, trachea, pancreas, liver, stomach, intestine and/or colon cells.

The three-dimensional cellular aggregate comprises an endoderm-like field of cells. The cells of the endoderm-like field of cells may express one or more of Gsc, Cdx2, Nedd9, Pyy Shh, Sorcs, Cer1, Sox17 and FoxA1. The cells of the endoderm-like field of cells may express Sox17, optionally wherein the cells of the endoderm-like field of cells express one or more of Gsc, Cdx2, Nedd9, Pyy, Shh, Sorcs, Cer1, and FoxA1. The endoderm-like field of cells may be arranged in one or more epithelial sheets or tube-like structures.

The one or more markers characteristic of mesodermal cells may be one or more genes the expression of which is characteristic of mesodermal cells. The one or more markers characteristic of mesodermal cells may be selected from, Bra, Meox1, Msgn, Osr1, Pax2, Raldh2, Ripply1/2, Tbx6, Tcf15, Uncx4.1, Kdr, and Pecam.

The one or more genes the expression of which is characteristic of mesodermal cells may be one or more genes the expression of which is characteristic of axial mesoderm, optionally wherein the one or more genes are selected from Bra, Chrd, FoxA2, Noto1 and Noggin.

The polarised three-dimensional cellular aggregate may comprise an axial mesoderm-like field of cells, optionally wherein the cells of the axial mesoderm-like field of cells express one or more of Bra, Chrd, FoxA2, Noto1 and Noggin.

The one or more genes the expression of which is characteristic of mesodermal cells may be one or more genes the expression of which is characteristic of paraxial mesoderm, optionally wherein the one or more genes are selected from Meox1, Msgn1, Tbx6, Tcf15, and Raldh2.

The polarised three-dimensional cellular aggregate may comprise a paraxial mesoderm-like field of cells, optionally wherein the cells of the paraxial mesoderm-like field of cells express one or more of Meox1, Msgn1, Tbx6, Tcf15, and Raldh2.

The polarised three-dimensional cellular aggregate may comprise neuromesodermal progenitor cells (NMPs), optionally wherein the neuromesodermal progenitor cells co-express Sox2, Bra and Nkx1.2.

The polarised three-dimensional cellular aggregate may comprise a tailbud-like region of cells in the posterior region, optionally wherein the cells of the tailbud-like region of cells express one or more of Bra, Cdx2, Cyp26a1, Fgf8, Fgf4, Wnt3a, and Wnt5a.

The one or more genes the expression of which is characteristic of mesodermal cells may be one or more genes the expression of which is characteristic of somitic mesoderm, optionally wherein the one or more genes are selected from Tcf15, Ripply1/2, Mesp1/2, Meox1, and Uncx4.

The polarised three-dimensional cellular aggregate may comprise a somitic mesoderm-like field of cells, optionally wherein the cells of the somitic mesoderm-like field of cells express one or more of Tcf15, Ripply1/2, Mesp1/2, Meox1, and Uncx4.

The polarised three-dimensional cellular aggregate may comprise one or more blocks of mesoderm, optionally wherein the cells of the mesodermal blocks express one or more of Tcf15, Ripply1/2, Mesp1/2, Meox1, and Uncx4.

The one or more genes the expression of which is characteristic of mesodermal cells may be one or more genes the expression of which is characteristic of intermediate mesoderm, optionally wherein the one or more genes are selected from Osr1 and Pax2.

The polarised three-dimensional cellular aggregate may comprise an intermediate mesoderm-like field of cells, optionally wherein the cells of the intermediate mesoderm-like field of cells express one or more of Osr1 and Pax2.

The one or more genes the expression of which is characteristic of mesodermal cells may be one or more genes the expression of which is characteristic of notochord, optionally wherein the one or more genes are selected from Bra, Noggin, Noto1, and FoxA2.

The three-dimensional cellular aggregate may comprise a cavitated structure, optionally wherein the cells of the cavitated structure express Gata6.

The three-dimensional cellular aggregate may comprise node-like cells, optionally wherein the node-like cells express one or more of Chordin, Gsc, Nodal, Lefty1/2, Noggin, Noto1, and FoxA2.

The polarised three-dimensional cellular aggregate may comprise a cluster of cells and wherein the cells of the cluster of cells express Nodal.

The one or more genes the expression of which is characteristic of mesodermal cells may be one or more genes the expression of which is characteristic of lateral plate mesoderm, optionally wherein the one or more genes are selected from Kdr, Pecam, Lefty 1/2, and Pitx2.

The polarised three-dimensional cellular aggregate may comprise a lateral plate mesoderm-like field of cells, optionally wherein the cells of the lateral plate mesoderm-like field of cells express one or more of Kdr, Pecam, Lefty 1/2, and Pitx2.

The polarised three-dimensional cellular aggregate may comprise a cranial mesoderm-like field of cells, optionally wherein the cells of the cranial mesoderm-like field of cells express one or more of Tbx1,

The polarised three-dimensional cellular aggregate may comprise a cardiac-like region of cells, optionally wherein the cells of the cardiac-like region of cells express one or more of Gata4, Gata6, Mesp1, Flk1, MIc2a IsI1, Pecam, cTnT, and Nkx2.5, optionally wherein the cardiac-like region of cells is located asymmetrically in the anterior region of the three-dimensional cellular aggregate.

The three-dimensional cellular aggregate may comprise a midline structure, optionally wherein the cells of the midline structure express Nodal.

The one or more markers characteristic of ectodermal cells may be one or more genes the expression of which is characteristic of ectodermal cells. The one or more markers characteristic of ectodermal cells may be one or more markers characteristic of neural or pre-neural cells. The one or more markers characteristic of neural or pre-neural cells may be one or more genes the expression of which is characteristic of neural or pre-neural cells, optionally wherein the one or more genes are selected from DII1, Hes5, Lnfg, Olig2, Pax3, Pax7, Sox1, Sox2, Irx3, Mnx1, Phox2a, Evx2, Ascii, Id2, and Lhx9.

The one or more markers characteristic of neural cells may be one or more markers characteristic of neural precursors. The one or more markers characteristic of neural precursors may be one or more genes the expression of which is characteristic of neural precursors, optionally wherein the genes are selected from DII1, Hes5, Olig2, Pax3, Pax7, Sox1 and Sox2

The one or more markers characteristic of neural cells may be one or more markers characteristic of differentiated neural precursor cells and/or may be one or more genes the expression of which is characteristic of differentiated neural precursor cells, optionally wherein the one or more genes are selected from Phox2a, Mnx1, Lhx9, Sox5, Nes.

The one or more markers characteristic of neural cells may be one or more markers characteristic of neural derivatives. The neural derivatives may be neurons and/or glial cells.

The polarised three-dimensional cellular aggregate may comprise neural crest-like cells, optionally wherein the neural crest-like cells express one or more of Sox5, Sox9, and Sox10.

The polarised three-dimensional cellular aggregate may comprise neuroectoderm-like region of cells, optionally wherein the cells of the neuroectoderm-like region express one or more of Sox1, Sox2, Olig2, and Pax7.

The polarised three-dimensional cellular aggregate may comprise neuronal cells, optionally wherein the neuronal cells express one or more of Phox2a and Mnx1.

The polarised three-dimensional cellular aggregate may comprise epithelial tracks or tubes, optionally wherein the cells of the epithelial tracks or tubes express Sox1.

The polarised three-dimensional cellular aggregate may comprise one or more placades For example, one or more sensory placades e.g. otic placodes and/or nasal placodes.

The polarised three-dimensional cellular aggregate may comprise anteriorly located cells at the border of neural and epidermal cells that will become otic and nasal placodes.

The polarised three-dimensional cellular aggregate may comprise anteriorly located cells at the border of neural and epidermal cells that will become otic and nasal placodes, optionally wherein the cells of the neuroectoderm-like region express one or more of Six2, Six3, Six 6, FoxG1, Eya1, Eya2, DIx5, Otx2, Pax1, Foxi2, Foxi3.

The invention provides a polarised three-dimensional cellular aggregate generated in vitro from one or more pluripotent stem cells, wherein:
(a) the polarised three-dimensional cellular aggregate comprises
   i. cells expressing one or more of Gsc, Cdx2, Nedd9, Pyy, Shh, Sores, Cer1, Sox17 and FoxA1,
   ii. cells expressing one or more of Bra, Meox1, Msgn, Osr1, Pax2, Pecam, Raldh2, Ripply1/2, Tbx6, Tcf15, Uncx4.1, Kdr, and Pecam, and
   iii. cells expressing DII1, Hes5, Lnfg, Olig2, Pax3, Pax7, Sox1, Sox2, Irx3, Mnx1, Phox2a, Evx2, Ascii , Id2, and Lhx9; and
(b) the polarised three-dimensional cellular aggregate is polarised along the anterior-posterior, dorsal-ventral and medio-lateral axes, and wherein
   i. the anterior-posterior axis is defined by at least an anterior region of cells and a posterior region of cells, wherein the cells of the anterior region express a higher or lower level of one or more genes than the cells of the posterior region,
   ii. the dorsal-ventral axis is defined by at least a dorsal region of cells and a ventral region of cells, wherein the cells of the dorsal region express a higher or lower level of one or more genes than the cells of the ventral region, and
   iii. the medio-lateral axis is defined by at least a medial region of cells and two lateral regions of cells, wherein the cells of the medial region express a higher or lower level of one or more genes than the cells of the lateral regions.

The invention provides a polarised three-dimensional cellular aggregate generated in vitro from one or more pluripotent stem cells, wherein:
(c) the polarised three-dimensional cellular aggregate comprises
   i. cells expressing one or more of Gsc, Cdx2, Nedd9, Pyy, Shh, Sores, Cer1, Sox17 and FoxA1,
   ii. cells expressing one or more of Bra, Meox1, Msgn, Osr1, Pax2, Pecam, Raldh2, Ripply1/2, Tbx6, Tcf15, Uncx4.1, Kdr, and Pecam, and
   iii. cells expressing DII1, Hes5, Lnfg, Olig2, Pax3, Pax7, Sox1, Sox2, Irx3, Mnx1, Phox2a, Evx2, Ascii , Id2, and Lhx9; and
(d) the polarised three-dimensional cellular aggregate is polarised along the anterior-posterior, dorsal-ventral and medio-lateral axes, and wherein
   i. the anterior-posterior axis is defined by at least an anterior region of cells and a posterior region of cells, wherein the cells of the posterior region express a higher level of Bra, Wnt3a and/or Cyp26a1 than the cells of the anterior region,
   ii. the dorsal-ventral axis is defined by at least a dorsal region of cells and a ventral region of cells, wherein the cells of the ventral region express a higher level of Shh than the cells of the dorsal region, and
   iii. the medio-lateral axis is defined by at least a medial region of cells and two lateral regions of cells, wherein the cells of the lateral regions express a higher level of Meox1 and/or Pax2 than the cells of the medial region.

The polarised three-dimensional cellular aggregate may comprise primordial germ cell-like cells (PGCs), optionally wherein the PGCs express Blimp1 and/or AP2g.

The three-dimensional cellular aggregate may comprise clusters of cells expressing Blimp 1 in the anterior region.

The polarised three-dimensional cellular aggregate may comprise cells that are placodal-like optionally wherein these placodal-like cells are sensory placodal-like.

The polarised three-dimensional cellular aggregate may comprise cells that become placodes optionally wherein they express one or more of Six2, Six3, Six 6, FoxG1, Eya1, Eya2, DIx5, Otx2, Pax1, Foxi2 and Foxi3.

The polarised three-dimensional cellular aggregate may comprise one or more of axial mesodermal derivatives, paraxial mesodermal derivatives, intermediate mesodermal derivatives and lateral plate mesodermal derivatives. The paraxial mesodermal derivatives may comprise somite cells. The intermediate mesodermal derivatives may comprise kidney cells and/or gonadal cells. The lateral plate mesodermal derivatives may be selected from one or more of cardiac cells, haematopoietic cells and limb cells.

The polarised three-dimensional cellular aggregate may comprise somite cells, kidney cells, gonadal cells, cardiac cells, haematopoietic cells and limb cells.

The polarised three-dimensional cellular aggregate may comprise at least 50 cells, at least 100 cells, at least 200 cells, at least 300 cells, at least 400 cells, at least 500 cells, at least 600 cells, at least 800 cells, at least 900 cells, at least 1000 cells, at least 1500 cells, at least 2000, at least 2500 cells, at least 5000 cells, at least 10,000 cells, at least 15,000 cells, at least 20,000 cells, at least 30,000 cells, at least 40,000 cells or at least 50,000 cells. Preferably, the polarised three-dimensional cellular aggregate comprises at least 20,000 cells. The polarised three-dimensional cellular aggregate may comprise 50-100,000 cells, 100-75,000 cells, 200-50,000 cells, 300-25,000 cells, 400-10,000 cells, 500-5,000 cells, 750-2,500 cells or 1000-2,000 cells. Preferably, the polarised three-dimensional cellular aggregate comprises 20,000-75,000 cells.

The polarised three-dimensional cellular aggregate may have a length of at least 0.05 mm, at least 0.1 mm, at least 0.2 mm, 0.3 mm, at least 0.4 mm, at least 0.5 mm, at least 0.6 mm, at least 0.7 mm, at least 0.8 mm, at least 0.9 mm, at least 1 mm or at least 1.5 mm. Preferably the polarised three-dimensional cellular aggregate has a length of at least 0.2 mm. The polarised three-dimensional cellular aggregate may have a length of 0.05-2 mm, 0.1-2 mm, 0.2-2 mm, 0.3-1.9 mm, 0.5-1.8 mm, 0.6-1.7 mm, 0.7-1.6 mm, 0.8-1.5 mm, 0.9-1.4 mm, 1.0-1.3 mm or 1.1-1.2 mm. Preferably, the polarised three-dimensional cellular aggregate has a length of 0.2-2 mm.

The polarised three-dimensional cellular aggregate may be elongate along the anterior-posterior axis. The anterior-posterior axis may be at least 5%, at least 10%, at least 15%, at least 20%, at least 25%, at least 30%, at least 35%, at least 40%, at least 45% or at least 50% longer than the dorso-ventral axis. Preferably, the anterior-posterior axis is at least at least 10% longer than the dorso-ventral axis.

The polarised three-dimensional cellular aggregate may be elongated from anterior to posterior. The diameter of the polarised three-dimensional cellular aggregate at the anterior end may be greater than the diameter of the polarised three-dimensional cellular aggregate at the posterior end.

The polarised three-dimensional cellular aggregate may be elongated along the anterior-posterior axis, optionally wherein the cells of the posterior region express a higher level of Bra than the cells of the posterior region.

The polarised three-dimensional cellular aggregate may have undergone one or more morphological elongation, optionally wherein the morphological elongations are convergent-extension and proliferation.

The polarised three-dimensional aggregate may comprise, within a Bra expressing region, an oval, polarized structure with differential adhesion between its cells that acts as a source of axial mesoderm.

The polarised three-dimensional cellular aggregate may comprise one or more of cavities, tubular structures, cysts pores, lumens, folds, plates, tracts, and segments.

The polarised three-dimensional cellular aggregate may have undergone one or more morphological shape changes, optionally wherein the morphological shape changes are one or more of elongation, cavitation, cyst formation and epithelialisation or segmentation.

The polarised three-dimensional cellular aggregate may have undergone one or multiple segmentations along the anteroposterior axis. The segments may be somite-like. The polarised three-dimensional cellular aggregate may have undergone bilaterally symmetrical budding at defined positions along the anteroposterior axis. The budding may be limb buds.

The polarised three-dimensional cellular aggregate may comprise one or more stem or progenitor cells or derivatives thereof. As used herein the term “progenitors” or “progenitor cells” refer to both stem cells and progenitor cells.

The polarised three-dimensional cellular aggregate may comprise haematopoietic progenitors or derivatives thereof. The haematopoietic progenitors or derivatives thereof may express one or more of Flk1, Scl, Runx1, Gata2, Cxcr4, cKit and CD41.

The polarised three-dimensional cellular aggregate may comprise one or more progenitors of the vascular system or derivatives thereof. The progenitors of the vascular system or derivatives thereof may express one or more of Flk1, Scl, Runx1, Gata2, Cxcr4, cKit and CD41.

The polarised three-dimensional cellular aggregate may comprise haematopoietic progenitors or derivatives thereof, optionally wherein these haematopoietic cells are haematopoietic stem cells or derivatives thereof.

The polarised three-dimensional cellular aggregate may comprise a vascular system.

The polarised three-dimensional cellular aggregate may comprise a vascular system, optionally wherein this is part of an aortic cluster.

The polarised three-dimensional cellular aggregate may comprise endothelial cells, optionally wherein the endothelial cells express one or more of VE-Cadherin, Flk1, Pecam and Scl.

The polarised three-dimensional cellular aggregate may comprise cysts comprising clusters of endothelial cells expressing one or more of VE-Cadherin, CD41, CD43 and CD45.

The haematopoietic progenitors may express one or more haemogloblin genes, optionally wherein the haemoglobin is fetal haemoglobin (HbF) or adult haemoglobin (HbA and HbB). The one or more haemogloblin genes may be Hbb (e.g. Hbb-bh1 and Hbb-y) or Hba (e.g. Hba-x).

The haematopoietic progenitors may express one or more markers of haematopoietic genes, optionally wherein the haematopoietic genes are selected from Flk1, CD41, cKit, CD45, CD31, Vecadh, Runx1, Spfi1, Gata2, Gata1, Scl1 and Tal1.

The haematopoietic progenitors derived from the polarised three-dimensional cellular aggregate may be capable of generating differentiated blood cells in vitro (e.g. as determined by a colony forming cell (CFC) assay), optionally wherein the differentiated blood cells are myeloid cells and/or lymphoid cells.

The myeloid cells may be selected from one or more of monocytes, macrophages, neutrophils, basophils, eosinophils, erythrocytes, megakaryocytes and platelets.

The lymphoid cells may be selected from one or more of T cells, B cells, and natural killer cells.

The polarised three-dimensional cellular aggregate may comprise one or more cardiac progenitor cells or derivatives thereof. The cardiac progenitor cells express one or more cardiac specific genes.

The polarised three-dimensional cellular aggregate may comprise a cardiac structure. The cardiac structure may be located in the anterior region of the polarised three-dimensional cellular aggregate, optionally wherein the cardiac structure is asymmetrically located in the anterior region of the three-dimensional cellular aggregate. The cardiac structure may comprise components of a vascular system, optionally wherein the cardiac structure comprises one or more blood vessels. The cardiac structure may comprise one or more cavities. The cardiac structure may comprise one or more tubular structures. The cardiac structure may beat or contract spontaneously. The cardiac structure may beat or contract at 10-250 beats per minute, 20-200 beats per minute, 30-175 beats per minute, 40-120 beats per minute, 50-100 beats per minute. The cardiac structure may start beating 6 or 7 days after the formation of the three-dimensional cellular aggregate.

The cardiac progenitor cells, or derivatives thereof, or cells of the cardiac structure may express at any point in their development one or more cardiac specific genes. The cardiac progenitor cells, or derivatives thereof, or the cells of the cardiac structure may express one or more cardiac specific genes. The one or more cardiac specific genes may be selected from Flk1, cTnT, MIc2a CD31, Mesp1, Nkx2-5, Tbx5, Tbx1, IsI1 and Pitx2. The cardiac structure may comprise a domain of cells expressing Flk1. The cardiac structure may comprise cells co-expressing cTnT, MIc2a CD31, and Nkx2-5. The cells of the cardiac structure may express Gata4 and/or Gata6.

The polarised three-dimensional cellular aggregate may be generated in vitro from one or more embryonic stem cells (ESCs). The embryonic stem cells may be naiive type embryonic stem cells, optionally wherein the naiive type embryonic stem cells are prepared by the 2i culture method. The embryonic stem cells may be non-naiive type embryonic stem cells, optionally wherein the non naiive type embryonic stem cells are prepared in ESLIF or medium comprising BMP and LIF.

The polarised three-dimensional cellular aggregate may be generated in vitro from one or more induced pluripotent stem cells (iPSCs).

The polarised three-dimensional cellular aggregate may be generated in vitro from one or more epiblast stem cells (EpiSCs).

The polarised three-dimensional cellular aggregate may be generated in vitro from one or more epiblast-like stem cells (Epi-like SCs). The polarised three-dimensional cellular aggregate may be generated in vitro from a single pluripotent stem cell.

The polarised three-dimensional cellular aggregate may be generated in vitro from a single colony derived from a single pluripotent stem cell.

The polarised three-dimensional cellular aggregate may be generated in vitro from one or more blastomeres derived from a pre-implantation epiblast.

The pluripotent stem cells may be mammalian pluripotent stem cells e.g. mouse pluripotent stem cells. The pluripotent stem cells may not be human pluripotent stem cells.

The invention provides a method for obtaining a polarised three-dimensional cellular aggregate, the method comprising:
(a) obtaining a cell suspension, wherein the cell suspension comprises one or more disassociated pluripotent stem cells;
(b) culturing the cell suspension under conditions that promote the transformation of at least one of the disassociated pluripotent stem cells into a three-dimensional cellular aggregate; and
(c) culturing the three-dimensional cellular aggregate under conditions that promote the transformation of the three-dimensional cellular aggregate into a polarised three-dimensional cellular aggregate; and
   wherein the polarised three-dimensional cellular aggregate is a polarised three-dimensional cellular aggregate as herein.

The invention provides a method for obtaining a polarised three-dimensional cellular aggregate, the method comprising:
(a) obtaining a cell suspension, wherein the cell suspension comprises one or more disassociated pluripotent stem cells;
(b) culturing the cell suspension under conditions that promote the transformation of at least one of the disassociated pluripotent stem cells into a three-dimensional cellular aggregate; and
(c) culturing the three-dimensional cellular aggregate under conditions that promote the transformation of the three-dimensional cellular aggregate into a polarised three-dimensional cellular aggregate; and
(d) culturing the polarised three-dimensional cellular aggregate under conditions that promote the differentiation of one or more cells of the polarised three-dimensional cellular aggregate.

The invention provides a method for obtaining a polarised three-dimensional cellular aggregate, the method comprising:
(a) obtaining a cell suspension, wherein the cell suspension comprises one or more disassociated pluripotent stem cells;
(b) culturing the cell suspension under conditions that promote the transformation of at least one of the disassociated pluripotent stem cells into a three-dimensional cellular aggregate;
(c) culturing the three-dimensional cellular aggregate under conditions that promote the transformation of the three-dimensional cellular aggregate into a polarised three-dimensional cellular aggregate; and
(d) culturing the polarised three-dimensional cellular aggregate under conditions that promote the differentiation of one or more cells of the polarised-three dimensional cellular aggregate into progenitor cells or derivatives thereof.

The invention provides a method for obtaining a polarised three-dimensional cellular aggregate, the method comprising:
(a) obtaining a cell suspension, wherein the cell suspension comprises one or more disassociated pluripotent stem cells;
(b) culturing the cell suspension under conditions that promote the transformation of at least one of the disassociated pluripotent stem cells into a three-dimensional cellular aggregate;
(c) culturing the three-dimensional cellular aggregate under conditions that promote the transformation of the three-dimensional cellular aggregate into a polarised three-dimensional cellular aggregate; and
(d) culturing the polarised three-dimensional cellular aggregate under conditions that promote the differentiation of one or more cells of the polarised three-dimensional cellular aggregate into stem/progenitors cells or derivatives thereof, organised into spatially organized regions.

The invention provides a method for obtaining a polarised three-dimensional cellular aggregate, the method comprising:
(a) obtaining a cell suspension, wherein the cell suspension comprises one or more disassociated pluripotent stem cells;
(b) culturing the cell suspension under conditions that promote the transformation of at least one of the disassociated pluripotent stem cells into a three-dimensional cellular aggregate;
(c) culturing the three-dimensional cellular aggregate under conditions that promote the transformation of the three-dimensional cellular aggregate into a polarised three-dimensional cellular aggregate;
(d) culturing the polarised three-dimensional cellular aggregate under conditions that promote the differentiation of one or more cells of the polarised three-dimensional cellular aggregate into stem/progenitors cells or derivatives thereof, organised into spatially organized regions; and
(e) isolating from the polarised three-dimensional cellular aggregate one or more progenitor cells or derivatives thereof.

A “cell suspension” as used herein refers to a suspension comprising single disassociated pluripotent stem cells i.e. a single cell suspension, and/or to a suspension comprising disassociated colonies comprising pluripotent stem cells i.e. a colony suspension (wherein a colony is derived from a single pluripotent stem cell).

Step (e) may comprise isolating one or more stem or progenitor cells or derivatives thereof, either as individual disassociated cells or as collections of cells. These collections of cells may comprise combinations of differentiated cell types, primordia, tissues or organs.

Step (b) may comprise culturing the cell suspension until one or more three-dimensional cellular aggregates is/are formed. Step (b) may comprise culturing the cell suspension for 5 minutes-48 hours, 10 minutes-24 hours, 30 minutes-12 hours, 1-6 hours or 2-4 hours. Preferably, step (b) comprise culturing the cell suspension for 1-24 hours.

Step (b) may comprise sorting the cell suspension (e.g. by flow cytometry) until the three-dimensional cellular aggregate is formed.

Step (c) may comprise culturing the three-dimensional cellular aggregate until one or more polarised three-dimensional cellular aggregates is/are formed. Step (c) may comprise culturing the three-dimensional cellular aggregate for 1-96 hours, 6-90 hours, 12-85 hours, 24-80 hours or 48-72 hours. Preferably, step (c) comprises culturing the three-dimensional cellular aggregate for 24-72 hours.

Step (d) may comprise culturing the polarised three-dimensional cellular aggregate until one or more progenitor cells or derivatives thereof is/are formed. Step (d) may comprise culturing the polarised three-dimensional cellular aggregate for 24-360 hours, 48-336 hours, 72-288 hours, 96-264 hours, 120-240 hours, 144-216 hours or 168-192 hours. Preferably, step (d) comprises culturing the polarised three-dimensional cellular aggregate for 48-96 hours.

Step (b) may comprise embedding the cell suspension in a gel and/or a matrix and culturing the cell suspension under conditions that promote the transformation of at least one of the disassociated pluripotent stem cells into a three-dimensional cellular aggregate.

Step (c) may comprise embedding the three-dimensional cellular aggregate in a gel and/or a matrix and culturing the three-dimensional cellular aggregate under conditions that promote the transformation of the three-dimensional cellular aggregate into a polarised three-dimensional cellular aggregate.

Step (d) may comprise embedding the polarised three-dimensional cellular aggregate in a gel and/or a matrix and culturing the polarised three-dimensional cellular aggregate under conditions that promote the differentiation of one or more cells of the polarised three-dimensional cellular aggregate.

Preferably the step of embedding promotes the formation of somite-like structures or segments.

In the methods, the gel or matrix may comprise at least one extracellular matrix protein or analogue thereof. The extracellular matrix protein may be one or more of collagen (e.g.

collagen IV), laminin, fibronectin, vitronectin and/or gelatin. Preferably, the extracellular matrix protein is collagen (e.g. collagen IV) and/or laminin. The matrix may activate signalling through β-integrin receptors. The gel may be a hydrogel. The gel may comprise or consist substantially of basement membrane matrix. The basement membrane matrix may comprise one or more of laminin, collagen (e.g. collagen IV), heparan sulphate proteoglycan and entactin. The gel may be formed from basement membrane extract, which may be isolated from a suitable basement membrane-secreting cell type, such as Engelbreth-Holm-Swarm (EHS) mouse sarcoma cells. Basement membrane extracts produced from EHS cells are commercially available under the trade names Matrigel (BD Biosciences, Franklin Lakes, N.J., USA), Cultrex (Trevigen Inc., Gaithersburg, Md., USA) and Geltrex (Invitrogen). Their major component is laminin, followed by collagen IV, heparan sulphate proteoglycan and entactin. Alternatively, the gel may be a polyacrylamide gel, e.g. a gel comprising across-linked polymer matrix formed by polymerisation of acrylamide and bis-acrylamide (e.g. N,N′-methylenebisacrylamide). Other suitable gel types may include alginate gels, polyethylene glycol (PEG)based gels and agarose gels.

In the methods, the polarised three-dimensional cellular aggregate may be cultured in the absence of extra-embryonic cells or tissue including primitive endoderm, amnion and/or trophoblast.

The methods may comprise a further step, prior to step (a), of culturing the cell suspension in 2i.

In the methods, the steps (c) and/or (d) may be performed on ultra-low adherence plates.

The one or more progenitor cells or derivatives thereof may be:
a. haematopoietic progenitor cells and/or derivatives thereof;
b. cardiac progenitor cells and/or derivatives thereof;
c. paraxial mesoderm and/or derivatives thereof;
d. somites and/or derivatives thereof (e.g. dermatome, myotome and/or sclerotome cells);
e. neural crest and/or derivatives thereof;
f. neural ectoderm and/or derivatives thereof (e.g. neural plate/tube cells and/or neurons);
g. placodal ectoderm and/or derivatives thereof (e.g. otic and/or nasal primordia);
h. intermediate mesoderm progenitor cells and/or derivatives thereof (e.g. renal and/or gonadal primordia);
i. axial mesoderm progenitor cells;
j. neuromesodermal progenitor cells and/or derivatives thereof (e.g. spinal cord neural progenitors and/or derivatives thereof, and/or paraxial mesoderm and/or derivatives thereof);
k. lateral plate mesoderm and/or derivatives thereof;
l. primordial germ cells and/or derivatives thereof;

m. node cells and/or derivatives thereof; and/or
n. endoderm and/or derivatives thereof (e.g. primordia for the oesophagus, stomach, intestine, lungs, pancreas, liver, trachea, thymus and/or thyroid).

The methods may comprise any of the following conditions:
a. to promote generation of neuromesodermal progenitors or derivatives thereof, a pulse of Chiron between 48 and 72 hours after aggregation, followed by culture in either N2B27 and BMP inhibitors (DMH1 or LDN193189) or N2B27 and Nodal with BMP inhibitors (DMH1 or or LDN193189);
b. to promote generation of paraxial mesoderm and derivatives thereof, a pulse of Chiron between 48 and 72 hours after aggregation, followed by culture in either N2B27 or N2B27 with FGF2 or FGF8 or N2B27 with BMP inhibitors (DMH1 or LDN193189);
c. to promote generation of somites and derivatives thereof, a pulse of Chiron between 48 and 72 hours after aggregation, followed by culture in either N2B27 or N2B27 with FGF2/FGF8 or N2B27 with FGF2/FG8 and BMP inhibitors (DMH1 or LDN193189);
d. to promote generation of neural crest and derivatives thereof, a pulse of Chiron and the ALK4, 5, 7 inhibitor SB431542 between 48 and 72 hours after aggregation, followed by culture in either N2B27 or FGF2/FGF8 or Chiron and FGF2/FGF8;
e. to promote generation of neural ectoderm and derivatives thereof; a pulse of Chiron between 48 and 72 hours after aggregation, followed by culture in either N2B27 or FGF2 or FGF8;
f. to promote generation of placodal ectoderm and derivatives thereof, a pulse of Chiron and the ALK4, 5, 7 inhibitor SB431542 between 48 and 72 hours after aggregation, followed by culture in either N2B27 or FGF2/FGF8 or Chiron and FGF2/FGF8;
g. to promote generation of intermediate mesoderm progenitor cells and derivatives thereof, a pulse of Chiron between 48 and 72 hours after aggregation, followed by culture in either N2B27 and BMP4;
h. to promote generation of axial mesoderm progenitor cells, a pulse of Chiron between 48 and 72 hours after aggregation, followed by culture in N2B27;
i. to promote generation of lateral plate mesoderm and derivatives thereof, a pulse of Chiron between 48 and 72 hours after aggregation, followed by culture in N2B27 with BMP4;
j. to promote generation of primordial germ cells and derivatives thereof, a pulse of Chiron together with BMP4 between 48 and 72 hours after aggregation, followed by culture in N2B27;
k. to promote generation of node cells and derivatives thereof, a pulse of Chiron between 48 and 72 hours after aggregation;
;. to promote generation of endoderm and derivatives thereof, a pulse of Activin A and Chiron between 48 and 72 hours after aggregation, followed by culture in Activin A.

The step of culturing the polarised three-dimensional cellular aggregate may comprise culturing the polarised three-dimensional cellular aggregate in a medium comprising FGF2 and VEGF. The progenitor cells or derivatives thereof may be haematopoietic progenitor cells or derivatives thereof, or cardiac progenitor cells or derivatives thereof.

The invention provides a method for obtaining a polarised three-dimensional cellular aggregate, the method comprising:
(a) obtaining a cell suspension, wherein the cell suspension comprises one or more disassociated pluripotent stem cells;
(b) culturing the cell suspension under conditions that promote the transformation of at least one of the disassociated pluripotent stem cells into a three-dimensional cellular aggregate;
(c) culturing the three-dimensional cellular aggregate under conditions that promote the transformation of the three-dimensional cellular aggregate into a polarised three-dimensional cellular aggregate; and
(d) culturing the polarised three-dimensional cellular aggregate under conditions that promote the differentiation of one or more cells of the polarised three-dimensional cellular aggregate into haematopoietic progenitor cells or derivatives thereof.

The haematopoietic progenitor cells or derivatives thereof may be asymmetrically-distributed in the polarised three-dimensional cellular aggregate.

The method may further comprise a pre-treatment step (performed before step (a)). The pre-treatment step may comprise culturing pluripotent stem cells in a medium comprising an activator of Wnt signalling and Leukaemia Inhibitory Factor (LIF). Optionally wherein the medium further comprises PDO3.

The method may further comprise a pre-treatment step (performed before step (a)). The pre-treatment step may comprise culturing pluripotent stem cells in a medium comprising an activator of Wnt signalling and an inhibitor of FGF signalling (e.g. PD03). Optionally wherein the medium further comprises Leukaemia Inhibitory Factor (LIF).

Step (b) may comprise culturing the cell suspension until one or more three-dimensional cellular aggregates is/are formed. Step (b) may comprise culturing the cell suspension for 5 minutes-48 hours, 10 minutes-24 hours, 30 minutes-12 hours, 1-6 hours or 2-4 hours. Preferably, step (b) comprise culturing the cell suspension for 1-24 hours.

Step (c) may comprise culturing the three-dimensional cellular aggregate until one or more polarised three-dimensional cellular aggregates is/are formed. Step (c) may comprise culturing the three-dimensional cellular aggregate for 1-96 hours, 6-90 hours, 12-85 hours, 24-80 hours or 48-72 hours. Preferably, step (c) comprises culturing the three-dimensional cellular aggregate for 24-72 hours.

The step of culturing the three-dimensional cellular aggregate (step (c)) may comprise culturing the three-dimensional cellular aggregate in a medium comprising an activator of Wnt signalling. The step of culturing the three-dimensional cellular aggregate (step (c)) may comprise culturing the three-dimensional cellular aggregate in a medium comprising an activator of Wnt signalling and an activator of SMAD signalling. The step of culturing the three-dimensional cellular aggregate (step (c)) may comprise culturing the three-dimensional cellular aggregate in a medium comprising an activator of Wnt signalling and an activator of Nodal/Activin signalling (e.g. Activin A). Preferably this step is performed on day 2 after formation of the three-dimensional cellular aggregate.

The step of culturing the polarised three-dimensional cellular aggregate (step (d)) is performed after step (c). Step (d) may comprise culturing the polarised three-dimensional cellular aggregate in a medium comprising FGF (e.g. FGF2) and VEGF. Step (d) may comprise culturing the polarised three-dimensional cellular aggregate in a medium comprising FGF (e.g. FGF2), VEGF and a further agent, wherein the further agent is a Shh signalling agonist (e.g. Shh, SAG (Smoothened Agonist)) and/or a BMP signalling antagonist (e.g. Noggin).

Step (d) may comprise culturing the polarised three-dimensional cellular aggregate until one or more haematopoietic progenitor cells or derivatives thereof is/are formed.

Step (d) may comprise:
i. culturing the polarised three-dimensional cellular aggregate in a medium comprising FGF (e.g. FGF2) and VEGF, optionally wherein the medium comprises FGF and VEGF from day 3 after formation of the three-dimensional cellular aggregate; and then
ii. culturing the polarised three-dimensional cellular aggregate in a medium comprising FGF (e.g. FGF2), VEGF and a further agent, wherein the further agent is a Shh signalling agonist (e.g. Shh, SAG (Smoothened Agonist)) and/or a BMP signalling antagonist (e.g. Noggin), optionally wherein the medium comprises FGF (e.g. FGF2), VEGF and the further agent from day 5 after formation of the three-dimensional cellular aggregate.

Step (d)(i) may comprise culturing the polarised three-dimensional cellular aggregate for 15 mins-72 hours, 30 minutes-66 hours, 1-60 hours, 6-54 hours, 12-48 hours, 18-42 hours or 24-36 hours. Preferably, (d)(i) comprises culturing the polarised three-dimensional cellular aggregate for 24-168 hours. Step (d)(ii) may comprise culturing the polarised three-dimensional cellular aggregate for 15 mins-72 hours, 30 minutes-66 hours, 1-60 hours, 6-54 hours, 12-48 hours, 18-42 hours or 24-36 hours. Preferably, (d)(ii) comprises culturing the polarised three-dimensional cellular aggregate for 24-144 hours.

In the method that promote the differentiation of one or more cells of the polarised three-dimensional cellular aggregate into haematopoietic progenitor cells or derivatives thereof, the steps of culturing typically comprise changing the media every 24 hours. The steps of culturing the three-dimensional cellular aggregate (step (c)) and/or the step of culturing the polarised three-dimensional cellular aggregate (step (d) may comprise performing a modified change of 25-75%, 30-70%, 35-65%, 40-60%, 45-55% or 50% of the media. Preferably, step (c) and/or step (d) comprise performing a change of 45-55% of the media.

The change in media may performed be once every 12-36 hours, once every 18-30 hours, once every 20-28 hours, once every 22-26 hours or once every 24 hours. Preferably, the change in media is performed once every 22-26 hours. The modified change of the media may start 24 hours, 36 hours, 48 hours, 60 hours, 72 hours, 84 hours, or 96 hours, 108 hours, or 120 hours after he formation of the three-dimensional cellular aggregate.

Preferably, the modified change of the media is started 72 hours (i.e. day 3) after the formation of the three-dimensional cellular aggregate.

The invention provides a method for obtaining a polarised three-dimensional cellular aggregate, the method comprising:
(a) obtaining a cell suspension, wherein the cell suspension comprises one or more disassociated pluripotent stem cells;
(b) culturing the cell suspension under conditions that promote the transformation of at least one of the disassociated pluripotent stem cells into a three-dimensional cellular aggregate;
(c) culturing the three-dimensional cellular aggregate under conditions that promote the transformation of the three-dimensional cellular aggregate into a polarised three-dimensional cellular aggregate; and
(d) culturing the polarised three-dimensional cellular aggregate under conditions that promote the differentiation of one or more cells of the polarised three-dimensional cellular aggregate into cardiac progenitor cells or derivatives thereof.

The step of culturing the three-dimensional cellular aggregate (step (c)) may comprise culturing the three-dimensional cellular aggregate in a medium comprising an activator of Wnt signalling.

Step (b) may comprise culturing the cell suspension until one or more three-dimensional cellular aggregates is/are formed. Step (b) may comprise culturing the cell suspension for 5 minutes-48 hours, 10 minutes-24 hours, 30 minutes-12 hours, 1-6 hours or 2-4 hours. Preferably, step (b) comprise culturing the cell suspension for 1-24 hours.

Step (c) may comprise culturing the three-dimensional cellular aggregate until one or more polarised three-dimensional cellular aggregates is/are formed. Step (c) may comprise culturing the three-dimensional cellular aggregate for 1-96 hours, 6-90 hours, 12-85 hours, 24-80 hours or 48- 72 hours. Preferably, step (c) comprises culturing the three-dimensional cellular aggregate for 24-72 hours.

The step of culturing the polarised three-dimensional cellular aggregate (step (d)) is performed after step (c). Step (d) may comprise culturing the polarised three-dimensional cellular aggregate in a medium comprising FGF (e.g. FGF2), VEGF and Ascorbic Acid. The polarised three-dimensional cellular aggregate may be cultured in a medium comprising FGF (e.g. FGF2), VEGF and Ascorbic Acid from day 4 after formation of the three-dimensional cellular aggregate (i.e. day 4 after aggregation). This step may be performed for at least 24 hours, at least 48 hours, at least 72 hours, at least 96 hours or at least 120 hours.

Step (d) may comprise culturing the polarised three-dimensional cellular aggregate until one or more cardiac progenitor cells or derivatives thereof is/are formed. Step (d) may comprise culturing the polarised three-dimensional cellular aggregate for 1-72, 6-66 hours, 12-48 hours, 24-36 hours, 12-144 hours, 24-144 hours or 24-168 hours. Preferably, step (d) comprise culturing the three-dimensional cellular aggregate for 24-168 h.

The invention provides a method for obtaining one or more progenitor cells or derivatives thereof, the method comprising:
(a) obtaining a cell suspension, wherein the cell suspension comprises one or more disassociated pluripotent stem cells;
(b) culturing the cell suspension under conditions that promote the transformation of at least one of the disassociated pluripotent stem cells into a three-dimensional cellular aggregate;
(c) culturing the three-dimensional cellular aggregate under conditions that promote thetransformation of the three-dimensional cellular aggregate into a polarised three-dimensional cellular aggregate; and
(d) culturing the polarised three-dimensional cellular aggregate under conditions that promote the differentiation of one or more cells of the polarised-three dimensional cellular aggregate into progenitor cells or derivatives thereof; and
(e) isolating from the polarised three-dimensional cellular aggregate one or more progenitor cells or derivatives thereof.

The invention provides a method for obtaining one or more progenitor cells or derivatives thereof, the method comprising:
(a) obtaining a polarised three-dimensional cellular aggregate as defined herein; and
(b) isolating from the polarised three-dimensional cellular aggregate one or more progenitor cells or derivatives thereof.

The progenitor cells or derivatives thereof may be any of the progenitor cells or derivatives thereof described herein.

In any of the methods described herein, the polarised three-dimensional cellular aggregate may be a polarised three-dimensional cellular aggregate as defined herein.

The step of culturing the three-dimensional cellular aggregate may comprise culturing the three-dimensional cellular aggregate in a medium comprising an activator of Wnt signalling. The activator of Wnt signalling may be any agent or molecule that activates the Wnt signalling pathway including the downstream signalling network The activator of Wnt signalling may be an activator of Wnt/β-catenin signalling. The activator of Wnt signalling may be a soluble protein. The activator of Wnt signalling may be a GSK inhibitor. The GSK inhibitor may be a GSK3 inhibitor, optionally wherein the GSK3 inhibitor is CHI99021 (Chi or Chiron). The activator of Wnt signalling may be selected from one or more of Wnt3, Wnt3a, Wnt5, Wnt8 and Wnt11.

The step of culturing the three-dimensional cellular aggregate (step (c)) may comprise shaking the three-dimensional cellular aggregate. Additionally or alternatively the step of culturing the polarised three-dimensional cellular aggregate (step (d)) may comprise shaking the polarised three-dimensional cellular aggregate. The shaking may be started after the formation of the three-dimensional cellular aggregate. The shaking may be started 12 hours, 24 hours, 36 hours, 48 hours, 60 hours, 72 hours, 84 hours, or 96 hours after the formation of the three-dimensional cellular aggregate. Preferably, the shaking is started 96 hours (i.e. day 4) after the formation of the three-dimensional cellular aggregate.

The steps of culturing typically comprise changing the media every 24 hours. The steps of culturing the three-dimensional cellular aggregate (step (c)) and/or the step of culturing the polarised three-dimensional cellular aggregate (step (d) may comprise performing a modified change of 25-75%, 30-70%, 35-65%, 40-60%, 45-55% or 50% of the media. Preferably, step (c) and/or step (d) comprise performing a change of 45-55% of the media. The change in media may performed be once every 12-36 hours, once every 18-30 hours, once every 20-28 hours, once every 22-26 hours or once every 24 hours. Preferably, the change in media is performed once every 22-26 hours. The modified change of the media may start 24 hours, 36 hours, 48 hours, 60 hours, 72 hours, 84 hours, or 96 hours, 108 hours, or 120 hours after he formation of the three-dimensional cellular aggregate.

Preferably, the modified change of the media is started 120 hours (i.e. day 5) after the formation of the three-dimensional cellular aggregate.

The one or more disassociated pluripotent stem cells may be one or more embryonic stem cells (ESCs).

The one or more disassociated pluripotent stem cells may be one or more induced pluripotent stem cells (iPSCs).

The one or more disassociated pluripotent stem cells may be a single pluripotent stem cell.

The one or more disassociated pluripotent stem cells may be a colony from a single pluripotent stem cell.

The one or more disassociated pluripotent stem cells may be one or more blastomeres from a pre-implantation epiblast.

The pluripotent stem cells may be mouse pluripotent stem cells. The pluripotent stem cells may not be human pluripotent stem cells.

One or more of steps (a)-(e) may be performed with the pluripotent stem cells in suspension, three-dimensional cellular aggregates in suspension and/or polarised three-dimensional cellular aggregates in suspension.

Step (a) may comprise growing one or more pluripotent stem cells on a solid substrate and then disassociating the pluripotent stem cells to obtain the cell suspension. The solid substrate may be a gelatin-coated, fibronectin-coated tissue substrate or may be comprised of a feeder-layer of cells, optionally mouse embryonic fibroblasts. The cells may be grown to 20-80% confluency, 30-70% confluency, or 40-60% confluency. Preferably, the cells are grown to 40-60% confluency.

Prior to the step of disassociating the pluripotent stem cells, the method may further comprise the step of culturing the pluripotent stem cells in a medium comprising one or more of an inhibitor of ERK signalling, an inhibitor of FGF signalling, an inhibitor of MEK signalling (e.g. PD03), an activator of BMP signalling, an activator of Wnt signalling and/or Leukaemia Inhibitory Factor.

The cell suspension may comprise 1×10³-1×10⁵ cells/ml, 5×10³-5×10⁴ cells/ml or 7.5×10³-2.5×10⁴ cells/ml.

Step (b) may comprise centrifugation of the one or more disassociated pluripotent stem cells, optionally wherein centrifugation of the one or more disassociated pluripotent stem cells initiates the formation of the three-dimensional cellular aggregate.

One or more of steps of the method (e.g. one or more of steps (b)-(d)) may be performed in a low or ultra-low adherence plate e.g. a low adherence 96 well plate.

Step (b) may comprise culturing the cell suspension for 24-72 hours, 30-66 hours, 36-60 hours, 42-54 hours, 44-52 hours, 46-50 hours, 47-49 hours or 48 hours.

Step (c) may comprise culturing the three-dimensional cellular aggregate for 1-48 hours, 6-42 hours, 12-36 hours, 18-30 hours, 20-28 hours, 22-26 hours, 23-25 hours or 24 hours.

Step (d) may comprise culturing the polarised three-dimensional cellular aggregate for 24-120 hours, 30-96 hours, 36-72 hours, 42-54 hours, 44-52 hours, 46-50 hours, 47-49 hours or 48 hours.

Step (b) may comprise culturing the cell suspension for 44-52 hours, step (c) may comprise culturing the three-dimensional cellular aggregate for 20-28 hours, and step (d) may comprise culturing the polarised three-dimensional cellular aggregate for 44-52 hours.

Step (b) may comprise culturing the cell suspension for 46-50 hours, step (c) may comprise culturing the three-dimensional cellular aggregate for 22-26 hours, and step (d) may comprise culturing the polarised three-dimensional cellular aggregate for 46-50 hours.

Step (b) may comprise culturing the cell suspension for 47-49 hours, step (c) may comprise culturing the three-dimensional cellular aggregate for 23-25 hours, and step (d) may comprise culturing the polarised three-dimensional cellular aggregate for 47-49 hours.

The cell suspension, three-dimensional cellular aggregate and polarised three-dimensional cellular aggregate may be cultured for a total of at least 120 hours, at least 130 hours, at least 140 hours, at least 150 hours, at least 160 hours, at least 170 hours, at least 180 hours, at least 190 hours, at least 200 hours, at least 210 hours, at least 220 hours, at least 230 hours, at least 240 hours or at least 250 hours.

One or more of steps of the method (e.g. one or more of steps (b)-(d)) may further comprise shaking the three-dimensional cellular aggregate or polarised three-dimensional cellular aggregate.

Step (b) may further comprise transferring one or more of the disassociated pluripotent stem cells into a well of a plate. The number of disassociated pluripotent stem cells transferred into a well of the plate may be 50-1000 disassociated pluripotent stem cells, 200-800 disassociated pluripotent stem cells, 300-800 disassociated pluripotent stem cells, or 400-600 disassociated pluripotent stem cells.

Step (d) may further comprise transferring the polarised three-dimensional aggregate into a well of a plate. Optionally, this could be a 6-well plate, 12-well plate, 24-well plate or 48-well plate.

The invention provides a method for obtaining a polarised three-dimensional cellular aggregate, the method comprising:
(a) culturing one or more epiblast pluripotent stem cells, wherein the step of culturing comprises
   i. culturing the epiblast pluripotent stem cells in a medium comprising FGF and Activin,
   ii. culturing the epiblast pluripotent stem cells in a medium comprising FGF, and
   iii. culturing the epiblast pluripotent stem cells in a medium comprising FGF and an activator of Wnt signalling;
(b) generating a cell suspension from the cultured epiblast pluripotent stem cells, wherein the cell suspension comprises one or more disassociated epiblast pluripotent stem cells; and
(c) culturing the cell suspension under conditions that promote the transformation of at least one of the disassociated epiblast pluripotent stem cells into a polarised three-dimensional cellular aggregate.

Step (a)(i) may comprise culturing the epiblast pluripotent stem cells in a medium comprising FGF and Activin, wherein the medium does not comprise an activator of Wnt signalling.

Step (a) (ii) may comprise culturing the epiblast pluripotent stem cells in a medium comprising FGF, wherein the medium does not comprise Activin and/or an activator of Wnt signalling.

Step (a)(iii) may comprise culturing the epiblast pluripotent stem cells in a medium comprising FGF and an activator of Wnt signalling, wherein the medium does not comprise Activin.

Step (c) may comprise culturing the cell suspension in a medium comprising an activator of Wnt signalling.

Step (c) may comprise culturing the cell suspension in a medium comprising an activator of Wnt signalling and FGF.

The activator of Wnt signalling may be any agent or molecule that activates the Wnt signalling pathway including the downstream signalling network. The activator of Wnt signalling may be an activator of Wnt/β-catenin signalling. The activator of Wnt signalling may be a soluble protein. The activator of Wnt signalling may be a GSK inhibitor. The GSK inhibitor may be a GSK3 inhibitor, optionally wherein the GSK3 inhibitor is CHI99021 (Chi or Chiron). The activator of Wnt signalling may be selected from one or more of Wnt3, Wnt3a, Wnt5, Wnt8 and Wnt11.

FGF may be FGF2 (or bFGF) FGF4 or FGF8 and/or FGF10. Activin may be Activin A or Nodal.

Step (a)(i) may comprise culturing the epiblast pluripotent stem cells for 6-42 hours, 12-36 hours, 18-30 hours, 20-28 hours, 22-26 hours, 23-25 hours or 24 hours.

Step (a)(ii) may comprise culturing the epiblast pluripotent stem cells for 6-42 hours, 12-36 hours, 18-30 hours, 20-28 hours, 22-26 hours, 23-25 hours or 24 hours.

Step (a)(iii) may comprise culturing the epiblast pluripotent stem cells for 6-42 hours, 12-36 hours, 18-30 hours, 20-28 hours, 22-26 hours, 23-25 hours or 24 hours.

Steps (a)(i)(iii) may each comprise culturing the epiblast pluripotent stem cells for 6-42 hours, 12-36 hours, 18-30 hours, 20-28 hours, 22-26 hours, 23-25 hours or 24 hours.

Step (c) may comprise culturing the cell suspension one or more epiblast pluripotent stem cells for 24-240 hours, 36-228 hours, 48-216 hours, 60-204 hours, 72-192 hours, 84-180 hours, 96-168 hours, 108-156 hours or 120-144 hours.

The one or more epiblast pluripotent stem cells may be a single epiblast pluripotent stem cell. The one or more epiblast pluripotent stem cells may be a single colony derived from a single pluripotent stem cell.

The epiblast pluripotent stem cells may be mouse epiblast pluripotent stem cells or epiblast-like pluripotent stem cells. The epiblast pluripotent stem cells may not be human epiblast pluripotent stem cells.

The invention provides a polarised three-dimensional cellular aggregate obtainable by any one of the methods described herein.

The invention provides a progenitor cell or derivative thereof obtainable by any one of the methods described herein. The invention further provides an organ and/or tissue comprising one or more progenitor cell or derivative thereof. The progenitor cell or derivative thereof may be any one of more of the progenitor cells or derivatives thereof described herein. The organ or tissue may be blood, vascular tissue, kidney, heart, lungs, somites, dermatome, myotome, sclerotome, neural crest, neural tube, neurons, sensory placode, gonad, notochord, neural-mesodermal progenitors, primordial germ cells, node, oesophagus, stomach, intestine, lungs, pancreas, liver, trachea, thymus and/or thyroid.

The polarised three-dimensional cellular aggregate may not comprise extra-embryonic cells or tissue including primitive endoderm, amnion and/or trophoblast. The polarised three-dimensional cellular aggregate may not be associated with extra-embryonic cells or tissue including primitive endoderm, amnion and/or trophoblast. The polarised three-dimensional cellular aggregate may not be associated with extra-embryonic cells or tissue including primitive endoderm, amnion and/or trophoblast. The polarised three-dimensional cellular aggregate may be unable to form yolk sac or placenta. The polarised three-dimensional cellular aggregate may not comprise yolk sac or placenta. The polarised three-dimensional cellular aggregate may lack any anterior neural derivatives. The polarised three-dimensional cellular aggregate may be unable to form brain tissue. The polarised three-dimensional cellular aggregate may not comprise brain tissue. The polarised three-dimensional cellular aggregate does not have the inherent capacity of developing into a human being.

### BRIEF DESCRIPTION OF THE DRAWINGS

The present disclosure will now be described, by way of example only, with reference to the accompanying drawings in which:

FIG. 1: Elongation of gastruloids. a. Schematic of the culture protocol. 200 to 300 ESCs were allowed to aggregate. The Wnt agonist CHIR99201 (Chi) was added between 48 h and 72 h after aggregation. Organoids were kept in suspension until 120 h (grey rectangle) and transferred into shaking cultures until 168 h. b. Three dimensional renderings and confocal sections of gastruloids at different times showing the elongation and expression of BRA, SOX2 and Gata6^{H2B-Venus} (green). c-d. Three-dimensional rendering (c, left panel) and confocal sections (c, central and right panels and d, zoom of tail region) of gastruloids at 168 h, showing the localization of CDX2, SOX2, SOX1 and BRA proteins. Scale bar: 150 μm. For each time point analyzed, the results reported were scored in at least 80% of the cases (n≥20). e. PCA analysis of RNAseq datasets using time-pooled gastruloids from 24 h to 168 h (2 replicates per time-point) and pooled mouse embryos at E6,5 (3 replicates), E7.8 (3 replicates), E8.5 (12-14 somites, 2 replicates) and E9.5 (ca. 24 somites, 2 replicates). For E7.8 embryos, the posterior half was used. For E8.5 and E9.5, the post-occipital embryonic domain was used. The dissected portion is colored in pale green. All autosomal genes were considered for this analysis. Principal Component 1 (PC1) shows a strong temporal component while PC2 discriminates between gastruloids or embryonic samples.

FIG. 2: Temporal patterns of gene expression in gastruloids. a. PCA of either pooled gastruloids during temporal progression from 24 h to 168 h (left), or murine embryos from E6.5 to E9.5 (right). The 100 top contributing genes to the first two principal components are overlaid, with those observed in both gastruloids and embryonic datasets shown in red text. b. Heatmap of scaled expression of genes associated with development of different embryonic structures in pooled gastruloids and embryos over time.

FIG. 3: Multi-axial organization of gastruloids. a-c. Gene expression in gastruloids at 144 h AA showing their axial organization. a. Wnt3a and Cyp26a1 expression (arrowhead) at the posterior end, where Raldh2 is not transcribed (empty arrowhead). Double FISH staining of Meox1 and Cyp26a1 (a, right-most panel) showing antero-posterior segregation of mesodermal precursors. b. Dorso-ventral (D-V) axis revealed by the ventral expression of Shh and Krt18, and of Lnfg dorsally (empty arrowheads). Double FISH staining of Sox2 and Shh confirmed a dorso-ventral segregation, with Shh expressed exclusively in endoderm precursors (b, right panel). C. Medio-lateral (M-L) axis of symmetry (dotted line) revealed by the bilateral expression of Meox1 and Pax2, complementary to the central distribution of Sox2 transcripts (empty arrowheads). For each gene, the proportion of gastruloids displaying the reported pattern is shown. Scale bar: 100 μm. d. 3D renderings of confocal stacks of 120 h gastruloids containing a Nodal reporter gene, stained for SOX2 (white) and BRA (red) proteins and imaged from the dorsal (left) and ventral (right) orientations; insets details of the posterior region. Reporter gene expression within the Bra expressing domain on the ventral surface is suggestive of a node-like structure (middle panel; FIG. 10). Additional expression of Nodal as a bilaterally asymmetric cluster of cells (white open arrow) is reminiscent of the asymmetric Nodal expression in the embryo (middle panel). Right panel shows a posterior view of the 3D rendering. e. Bar graph showing the frequency distribution of asymmetric and symmetric expression of Nodal or Meox1 in 120 h gastruloids. ###(observed versus expected frequency, based on the embryonic gene expression pattern): p<0.0001; **(observed versus expected frequency in asymmetric Nodal expression based on the frequency of Meox1 asymmetry in gastruloids).

FIG. 4: Collinear Hox gene expression in gastruloids. a. PCA plot solely based on Hox transcripts datasets extracted from pooled gastruloid and embryonic data across time points. Replicate batches of organoids primarily cluster according to their age at collection. b. Transcript profiles over the HoxA cluster, using time-sequenced pooled gastruloids. A progressive wave of transcription through Hoxa genes is observed between the 72 h and 168 h time-points. c. In situ hybridizations of 168 h gastruloids using probes for various Hoxd genes. Expression becomes spatially restricted along the A-P axis along with the respective position of the genes in the cluster. For each gene, the proportion of gastruloids displaying the reported expression pattern is shown in the bottom right corner of the image, expressed as a fraction of the total number of gastruloids analyzed. Scale bar: 100 μm. d. Double FISH staining of Hoxd4 with Sox2 or Meox1 (respectively marking the neural and mesodermal precursors) showed that Hoxd4 expression colocalized with both markers, suggesting that gastruloid implement both neural and mesodermal Hoxd gene expression. Scale bar: 200 μm.

FIG. 5. a-d. Gastruloids produced using Gata6^{H2BVenus} mESCs treated with a pulse of the GSK3 inhibitor Chiron between 48 h and 72 h AA and fixed either at 48 hh (a), 72 (b), 96 h (c) or 120 h (d) and imaged by confocal microscopy. BRA and SOX2 proteins are stained in red and white, respectively. VENUS signal (green) reports Gata6 expression and Hoechst (Blue) marks the nuclei. Gastruloids corresponding to the 3D renderings shown in FIG. 1a. Each fluorescent channel is displayed to the right of each merged image. Gata6 or Gata6 and SOX2 signals were undetectable in a and b, respectively, and therefore not shown. Three z-sections are shown for each gastruloid. The bright field outline of each gastruloid is indicated by the dashed lines. Scale-bars as indicated.

FIG. 6. a. Heat map showing the temporal evolution of 97 out of the 250 most variable genes throughout embryonic development from E6.5 to E9.5 (left) and their corresponding expression over the gastruloid time-course, from 24 h to 168 h (right). Expression levels are highlighted by color scale from blue to red (bottom left). Genes were clustered according to their expression behaviour in the embryo and enriched GO term categories were identified for each cluster by using the Gorilla and Revigo tools. Finally, a functional classification of each cluster was established based on the identified GO term categories and literature-based evidences. b. Expression of markers for different embryonic tissues through the gastruloid time-course. The two replicates of each time point are represented by triangles and circles. The black dotted line in each plot represents the average behaviour of the different genes displayed in the plot. c. PCA analysis of RNAseq datasets from either pooled or individual gastruloids using the top 1000 most highly expressed genes. Despite different strategies used for pooled versus individual gastruloids RNAseq (accounting for the sample segregation across PC1), the clustering of pooled and single-gastruloid datasets illustrates both the homogeneity of gastruloid cultures and the representativeness of pooled samples to single gastruloid samples.

FIG. 7: Gastruloids display spatio-temporal organization in the expression profiles of neural, mesodermal and endodermal marker genes. a-f. The expression profiles of several genes normally expressed in the embryonic neural, mesodermal and endodermal domains was analyzed by plotting the RNAseq data in pooled gastruloids in heatmaps of scaled gene expression (a, c, e) and/or by WISH (b, d, f). a-b. Expression of different neural markers was detected in our RNAseq. Genes like Lnfg or Irx3 were detected forming continuous and homogenous domains located in the central and dorsal portion of the gastruloids, reminiscent of their expression domains in the embryo (b, upper panels).

Instead, genes involved in notch signaling in neural progenitors (Hes5, Dll1) and in the terminal differentiation of neural precursor (Phox2a, Mnx1) displayed a salt and pepper expression pattern, consistent with the lack of an organized neural tube structure (FIGS. 8-10). However, the latter mRNAs also displayed a graded distribution along the anterior to posterior extension of the gastruloid axis and were absent from its posterior half (empty red arrowheads). c-d. Genes normally expressed in different types of mesoderm precursors in the embryo (e.g. Tcf15 in paraxial somatic mesoderm, Osr1 in intermediate mesoderm, Bra in notochord and PSM and Pecam in lateral plate mesoderm) were expressed in reproducible and spatially restricted domains within the gastruloids. e-f. Endoderm specific genes were also expressed in gastruloids. Particularly, genes expressed in the embryonic digestive tract were consistently found on the ventral side of gastruloids. For each gene, the proportion of gastruloids displaying the reported expression pattern is shown in the upper right corner of the image, expressed as a fraction of the total number. Scale bar: 100 μm.

FIG. 8. a. Gastruloids formed from Sox1^{GFP} (green); Bra^{mCherry} (red) mESCs were fixed at 168 h AA and stained for SOX2. White arrowheads indicate tubular SOX2/SOX1 positive neural structures. Red arrowheads point to the presumptive digestive tube. b. Gastruloids at 144 h were WISHed for Sox2 and Meox1 antisense probes, cryo-sectioned in 8 μm thick transversal cross-sections and counter-stained with Nuclear Fast Red. Sox2 positive cells localized predominately in a compact dorsal domain, whereas Meox1 signals was found in two bilateral domains. The domain of expression of each gene is delimited with white dashed lines. c. Haematoxylin-Eosin staining of transversal paraffin sections of different gastruloids at 120 h AA, showing the cell type diversity and several degrees of tissue organization. d. Gastruloids formed from SOX1^{GFP} (green); Bra^{mCherry} (red) mESCs were fixed and stained at 168 h for OLIG2 (top-panel, white), PAX3 (mid-panel, red) and PAX7 (lower-panel, red). Scale bars as indicated. c, d. Gastruloids formed from Sox1^{GFP} (green); Bra^{mCherry} (red) mESCs collected at 168 h AA and stained for SOX17 (magenta in c) or CDX2 (magenta in d). Scale bars as indicated.

FIG. 9. a-e. Double FISH staining of gastruloids from Sox1^{GFP} (green); Bra^{mCherry} (red) mESCs at 144 h with Meox1 and Cyp26a1 (a), Sox2 and Shh (b), Sox2 and Meox1 (c), Meox1 and Hoxd4 (d) or Sox2 and Hoxd4 (e). Scale bar: 200 μm

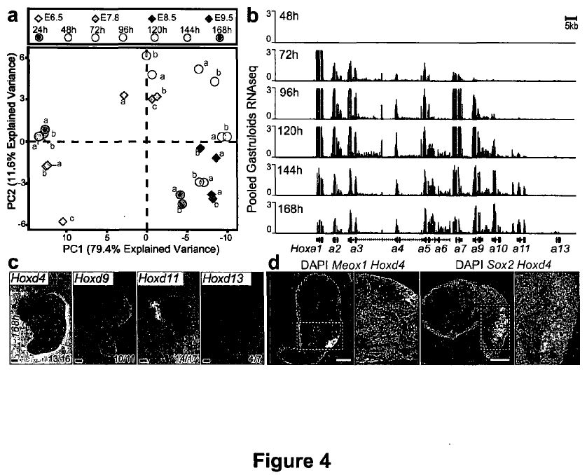

FIG. 10. a-b. Gastruloids formed from Nodal mESCs were fixed at 120 h AA. They were stained for CDX2, YFP (Nodal^{YFP}) and E-Cadherin (a, top panel), CDX2, YFP (Nodal^{YFP}; green) and Phalloidin (a, bottom panel) or CDX2, YFP and E-CADHERIN (both with an Alexa-488 secondary antibody) and SOX2 (b). Maximum intensity projection of a representative gastruloid in b, with the node-like structure highlighted. Hoechst marks the nuclei (greyscale in a, blue in b). c-d. In situ hybridizations of gastruloids at different time-points AA. Asterisk in d mark the presumptive node-like cells. White arrowheads point towards Nodal expressing cells distributed asymmetrically, on the lateral side of the gastruloid. e. Three dimensional renderings of confocal stacks of 120 h gastruloids containing a Nodal reporter gene (green) and stained for SOX2 (white) and BRA (red) proteins. SOX2 signal identifies dorsal cells. Left and right panels show the same gastruloid, imaged from two different polar directions i.e. top (dorsal) and bottom (ventral) or ‘left’ and ‘right’ depending on the orientation of the gastruloid. Insets in specific panels show a cross-section through the gastruloid at the indicated z plane. White arrowheads indicate the region of biased Nodal expression. Empty white arrowheads point to the node-like cells marked by the Nodal reporter gene. See also FIG. 4d. f. In situ hybridizations of gastruloids 120 h (left) and 144 h AA (right). The midline of the gastruloid is marked by a dashed white line. White arrowheads point towards Nodal expressing cells distributed asymetrically on the lateral side of the gastruloid. In c, d, and f, the proportion of gastruloids displaying the reported expression pattern is shown in the bottom left corner of each image, expressed as a fraction of the total number.

FIG. 11. a, b. Dorsal (a) and ventral (b) sections of the same representative gastruloid shown in the 3D renderings in FIG. 4d, fixed at 72 h AA and stained at 120 h for Nodal (green), BRA (red) and SOX2 (white). Hoechst was used to mark the nuclei. Scale bar indicates 100 μm.

FIG. 12. a. Heatmap of unscaled gene expression in E6.5-E9.5 mouse embryos, showing Hox gene transcript levels over time. b. RNAseq mapping showing Hoxa and Hoxd gene expression in these embryos. After a first wave of transcription of 5′ Hoxa and Hoxd genes, likely reflecting their activation in extraembryonic tissues, the HoxA and HoxD clusters were progressively transcribed from E7.8 until E9.5 when expression of Hox13 paralogs was detected. c. Heatmap of unscaled gene expression in pooled gastruloids, showing Hox gene transcript levels over time. d. RNAseq mapping showing Hoxd gene expression in pooled gastruloids at different time points. Hoxd genes sub-groups are progressively activated starting at 72 h until 168 h AA, when expression of Hoxd13 starts to be detected (e), thus resembling the temporal activation described in vivo (a, b). e. Whole mount in situ hybridization of gastruloids collected at different time points and showing the detectable initiation of different Hoxd genes expression. Each panel report the earliest stage where transcripts of the corresponding gene were detected (black arrowhead). Expression of Hoxd4 was already strong at 96 h AA indicating that its transcripts are rapidly upregulated compared to Hoxd9 which is faintly expressed at this stage. Scale bar: 100 μm.

FIG. 13. a. Principal component analysis (PCA) based on Hox transcripts datasets only, extracted from individually sequenced gastruloids across time points (10 individual organoids per time point). The analysis was carried out using the Loge transformed FPKM+1 value of all 39 Hox genes. Replicate batches of organoids primarily cluster according to their age at collection. The clustering revealed the low sample-to-sample variation. Instead, replicates were clearly separated through the temporal parameter, representing 93.6% of total sample variation. b. Comparison of Hoxa (top panel) and Hoxd (bottom panel) gene expression profiles amongst individual gastruloids confirmed the low inter-sample variation among time-points, illustrated with the 120 h condition. c. Whole mount in situ hybridization of 168 h AA gastruloids showing the expression of different Hoxa paralogs. The proportion of gastruloids displaying the reported expression pattern is shown in the upper right corner of the image, expressed as a fraction of the total number. Scale bar: 100 μm.

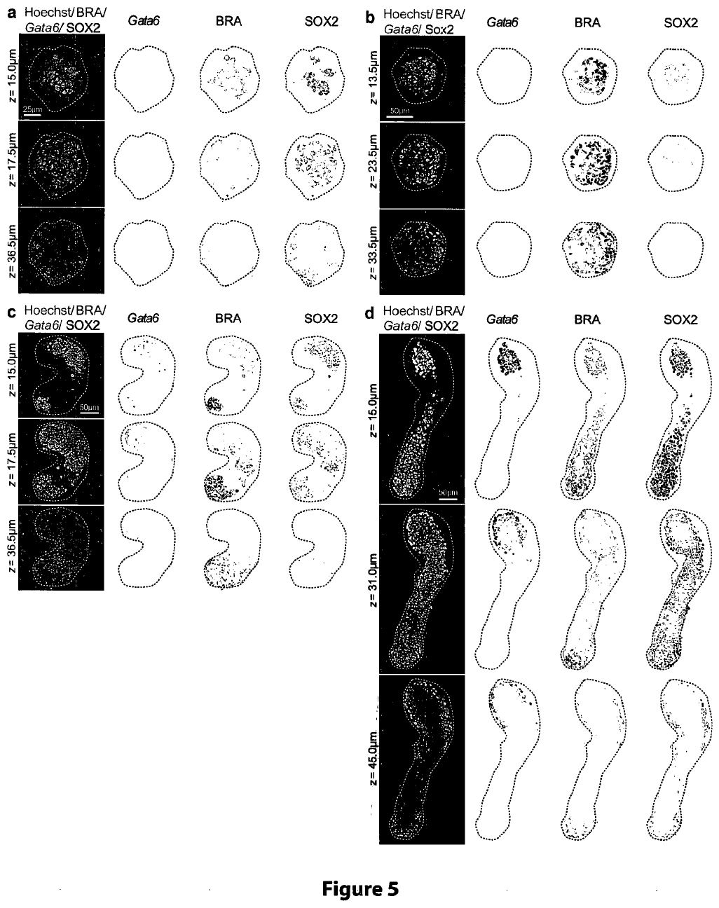

FIG. 14. a. Dot plot representing the progression in the measured longitudinal extension of gastruloids produced either from ES or from iPS cells. b. Light microscopy images showing representative examples of gastruloids at the different time-points analysed in (a). Zoom: 10×. Note that iPS derived gastruloid display delay in their longitudinal extension rate and at 120 h AA, they are significantly smaller than their ESC-derived counterparts. For this analysis, gastruloids were produced starting from the same number of cells (800 cells per well). c. Dot plots representing the mRNA levels of Bra, showing comparable dynamics of this gene in both types of gastruloids. d. Confocal images showing the expression of Oct4, SOX2 and BRA (upper panel) or of Oct4, SOX1 and CDX2 (bottom panel) in 120 h gastruloids derived from the iPS cell line Oct4::Gfp (IpSL40N). iPS-derived gastruloids were fixed at 120 h AA and stained for SOX2-BRA (left) and CDX2-SOX1 (right). Oct4::GFP signal is shown in greys. Scale bars: 200 μm. e. Dot plots representing the mRNA levels of Hoxd genes in ES or iPS cell-derived gastruloids collected at different time-points AA. Both gastruloids sequentially activated Hoxd gene expression. However, their temporal activation seemed to be delayed in iPS gastruloids (especially that of the most 3′ Hoxd paralogs). e. Whole mount in situ hybridization of 144 h AA gastruloids, showing the expression of different Hoxd paralogs. Even though iPS derived gastruloids reproduced the antero-posterior Hoxd collinear expression, Hoxd9 expression domain often extended more anteriorly when compared to ESs cell derived gastruloids (see FIG. 5e), occupying approximately the same domain than Hoxd4. Also patches of Hoxd negative cells were often found within the Hoxd4/Hoxd9 expression domain (white asterisks). Scale bar: 100 μm.

FIG. 15: Development of an improved protocol to generate gastruloid-based cardiac organogenesis models. (A) Scheme of the improved protocol to generate gastruloid-based cardiac organogenesis models. N2B27 represent the classic medium, while N2B27+++ is the medium with the addition of VEGF, bFGF and AA; (B) Representative pictures showing the development of beating structures in the anterior gastruloid portion. Red and white lines highlight the beating portion borders at time zero (red) and after 10 msec (white); (C) Quantification of the number of gastruloids developing a beating portion in absence (N2B27) or presence (N2B27+++) of the organ specifying factors; (D) Bright-field (upper panel) and Z-stack maximum intensity projection (lower panel) showing cardiac troponin T (cTnT) expression in gastruloids; (E) RT-qPCR analysis of the cardiac progenitor marker Mesp1, the early cardiac differentiation marker Nkx2.5 and the differentiated cardiomyocyte marker a-actinin in mESCs and gastruloids from Day 3 to Day 7.

FIG. 16: Development of an endothelial-like network in Gastruloids. (A) Analysis of Flk1 expression in gastruloids from a Flk1-GFP mESC reporter line from Day 4 to Day 7 in culture; (B) Z-stack maximum intensity projection showing the expression of Flk1 and cardiac troponin T in gastruloids from a Flk1-GFP mESC reporter line at day 7; (C) Z-stack maximum intensity projection showing co-localization of Flk1 and CD31/PECAM1 in gastruloids from a Flk1-GFP mESC reporter line at day 7.

FIG. 17: Production of haematopoietic cells from mouse ES gastruloid cultures in the presence of growth factors. A. Schematic representation of the haematopoietic-promoting gastruloid culture. ES cells are routinely kept in serum and LIF containing conditions, and passaged once in LIF and 2i prior to establishment of the culture in low adherence plates. After a 24 h-pulse with Activin and Chiron (48-72 h), gastruloids can be differentiated in FGF2+VEGF for up to 10 days with half-medium changes. When additional cytokines are added 75% of the medium is removed, and cytokines added in 150 ul of fresh medium for a total final volume of 200 ul. Different combinations of SCF, Shh and Noggin have been tested from day 5 onwards, with promising results SHH±SCF. B. Bright-field (top) and fluorescent (bottom) microscopy images of 3D gastruloid cultures at different days of differentiation in the presence of FGF2 and VEGF. C. Gene expression changes in day 5 gastruloids produced in Act/Chi and FGF2/VEGF conditions, and compared to the original ES cells. Pou5f1, Sox2 -pluripotency; Sox17 -endoderm; Runx1, Spfi1, Gata2, Gata1, Tal1—haematopoiesis; Flkl, Vecadh—endothelial (data summarises n=2 independent experiments); note that the relative gene expression changes are represented in logarithmic scale. D. Expression of both embryonic (Hbb-bhl) and adult (Hbb-bl) hemoglobin are enhanced in day 6 gastruloids by the FGF2/VEGF protocol; mean±SD of 3 independent experiments. E. Presence of haematopoietic progenitors in day 6 gastruloid cultures, which is strongly enhanced by the addition FGF2/VEGF during gastruloid formation (n=2, each with 2 technical replicates; mean±SD).

FIG. 18: Development of gastruloid culture conditions to enhance generation of early haematopoietic percursors. A. Representative flow cytometry plot showing the presence of CD41+CKit+cells within Flk1-GFP-expressing cells in day 6 gastruloids obtained in the presence of FGF2 and VEGF. B. The same populations are enhanced through addition of Shh alone (representative plot) or in combination with SCF (not shown). C. Representative mixed erythroid/myeloid colony reflecting the presence of multlineage progenitors in day 6 gastruloid cultures upon addition of Shh.

FIG. 19: Gastruloids can express markers of both first and second heart fields. (A-C) RT-qPCR graphs showing relative fold expression of first (Tbx5, C) and second (IsI1 and Tbx1, A, B) heart field markers in Gastruloids from 72 to 168 hours, compared to undifferentiated mESCs. GAPDH was used to normalize data. (D-F) Hematoxylin and eosin staining (D) and immunofluorescence (E-F) on consecutive 168 hours Gastruloid sections showing expression of Tbx1 in green (E′, E″, F, F″, F′″), Tbx5 in magenta (E, E″) and IsI1/2 in magenta (F, F′, F′″). Note that the area of Tbx5 expression (dashed line) is close but mutually exclusive with the area of Tbx1/IsI1-2 expression (arrowhead). Dapi was used to stain nuclei (blue). Scale bars 100 μm.

FIG. 20: The formation of a beating portion in Gastruloids passes through a cardiac crescent-like domain

(A-D) Immunofluorescence for cTnT (magenta) on non-beating (A, B) and beating Gastruloids (C, D) at 144 (A-C) and 168 (D) hours. Note that Gastruloids initially show a crescent-like domain of cTnT expression (A, B), reminiscent of the E7.5 cardiac crescent stage of mouse embryos. The expression of cTnT then progressively increases (C) and becomes more restricted to the beating portion (D). The bottom panel shows a representative picture (E) and its 3D reconstruction through the IMARIS software (F). Dapi was used to stain nuclei (blue). Scale bar 100 μm.

FIG. 21: Aortic clusters visible from prolonged culturing of gastruloids. (A) A section through the Aorta-gonad-mesonephros (AGM) of the E11.5 mouse embryo, showing the morphology of the aorta (Ao), and gene expression of the intra-aortic cluster which gives rise to the Haematopoietic Stem Cells. CV; Caudal Vein, UGR; Urogenital ridges. Taken from Medvinsky et. al. (2011). (B) Confocal images of 192 hour gastruloids cultured in the presence of VEGF and FGF2 show evidence of a cavitated structure positive for CD31 (yellow) with an intra-cavity cluster of cells that are cKit (blue) and CD45 (magenta) positive, suggesting that they are HSC-like.

FIG. 22: Transplantation of HSC-like gastruloid-derived cells to mouse. (A-D) Flow cytometric analysis of CD45.1 and CD45.2 cells from a week 9 mouse bone-marrow, following injection of unsorted populations of VEGF-treated gastruloids (A), unsorted populations of Shh-treated gastruloids (B), CD45+ sorted populations of VEGF-treated gastruloids (C) and control mice without injection (D), showing an increased engraftment of CD45.1+;CD45.2+ population of cells in the gastruloid injected animals compared to the control.

FIG. 23: Examples are shown of the of embedding gastruloids in 75% matrigel.

FIG. 24: Examples of 144 hrs old gastruloids embedded in Matrigel.

FIG. 25: Impact of preculture conditions on the frequency of elongated gastruloids. Left, quantification of different kinds of treatment before aggregation with two different starting numbers of cells. Right, some examples of a typical experiment at 120 hrs after aggregation.

FIG. 26: PGC-like cell formation in gastruloids. (a) Co-expression of Blimpl-GFP and AP2gamma in 120 h gastruloids made from the Blimpl-GFP cell line. (b) Flow cytometric analysis of Blimpl-GFP gastruloids at 120 h, showing co-expression of beta-Integrin, SSEA1 and Blimp1-GFP. (c) Gastruloids made from the Stella-GFP,Esg1-tomato (SGET) cell line at 120 h. (d) Flow cytometric analysis of co-expression of Stella-GFP and Esg1-Tomato in 120 h SGET gastruloids. (e) Co-expression of Stella and AP2gamma in 120 h SGET gastruloids.

FIG. 27: Embryonic organoids recapitulate heart organogenesis. a, Gastruloids form a beating portion on their anterior side at 168 h. White and red lines highlight displacement of the beating domain over 10 msec. b, Frequency of beating structures at different time points. n=6 independent experiments. c, Immunofluorescence for Gata4 and cTnT on gastruloids at 168 h. d-f Calcium imaging (d), representative spiking profile (e) and spiking frequency (f) of the gastruloid cardiac portion at 168 h. n=21 gastruloids. g-k, qPCR gene expression profiles of cardiac genes in gastruloids from 96 to 168 h. Data are expressed as relative fold expression compared to mESCs. n=4 independent experiments. I, Spatial localization of Mesp1⁺ cells (stained for GFP) at 96 h, compared to Brachyury expression. m, Tracking of Mesp1+ cells from 96 to 110 h. Red: tracked cells; light blue: cell tracks forward. n, Spatial localization of Gata6⁺ cells at 96 and 120 h, compared to Brachyury expression. Scale bars, 100 μm. A, anterior; P, posterior. +++, N2B27 with cardiogenic factors.

FIG. 28: Development of a vascular-like network. a, b, Spatial localization of Flk1⁺ cells at 96 (a) and 120 h (b), compared to Brachyury expression. c, Light sheet live imaging of the anterior portion of Flk1-GFP gastruloids from 120 to 144 h, highlighting the formation of a vascular-like network. d, The Flk⁺ vascular-like network is positive for CD31. e, angiogenesis assay showing tube formation in isolated Flier cells, compared to HUVEC. Scale bars, 100 μm. A, anterior; P, posterior.

FIG. 29: Embryonic organoids form a first and a second heart field. a-c, Expression levels of markers of FHF (a) and SHF (b) in gastruloids from 96 to 168 h. Data are expressed as relative fold expression compared to mESCs. n=4 independent experiments. d, RNA-scope showing spatial localization of FHF and SHF domains. e, Representative plot showing the gating strategy used to isolate SHF-enriched progenitors. f-k, Expression levels of markers of SHF (f-h) and FHF (i-k) in Gata6⁺/CXGR⁻ and Gata6⁺/CXGR⁺ cells isolated from gastruloids at 168 h. Data are expressed as relative fold expression compared to Gata6⁺/GXCR⁻ cells. n=4 independent experiments. Scale bar, 100 μm. A, anterior; P, posterior.

FIG. 30: Embryonic organoids recapitulate cardiac morphogenesis. a, Light sheet imaging of cleared gastruloids from 144 to 168 h show an initial crescent-like domain which is condensed in a beating bud at 168 h. b, Schematic illustrating the comparison between gastruloid stages of cardiac development and embryonic stages from cardiac crescent to linear heart tube. c, Quantification of geometrical properties (spareness) of the gastruloid cardiac domains compared to those of defined artificial shapes. n=31 gastruloids at 144 h and n=27 gastruloids at 168 h. d,e, the cardiac domain is localized in close proximity to the anterior epithelial gut tube-like structure, separated by a CD31⁺ endocardial layer. Scale bar, 100 μm. A, anterior; P, posterior.

FIG. 31: Comparison between N2B27 and N2B27+++ culture conditions. a, Schematics of the different culture conditions tested. b-e, Percentage of beating gastruloids from 120 to 168 h in the different conditions. f, Exposure to N2B27+++ induces beating at higher frequencies compared to exposure to single factors or couples. g, h Frequences of beating gastruloids grown in 96 well plates (g) and comparison with those grown in 24 well plates from 144 h (h). Each dot in b-h represent an independent experiment. i-j, quantification of area and axis at 168 h of gastruloids grown in N2B27 or N2B27+++. Mean of n=3 independent experiments.

FIG. 32: Development of the cardiac portion of gastruloids. a-c, Treatment of 168 h gastruloids with Nifedipine (n=9 gastruloids) (a) or Isoproterenol (b, c) abolish or fastens calcium spiking in the gastruloid cardiac portion, respectively. Graph in (b) shows representative calcium spikes of gastruloids before and after Isoproterenol treatment, and graph in (c) the percentage of increase over baseline frequency of gastruloids after Isoproterenol treatment. n=24 gastruloids d, e, FACS analysis of Mesp1-GFP gastruloids from 96 to 168 (d) and relative quantification (e). n=2 independent experiments. f, g, FAGS analysis of Gata6-Venus gastruloids from 96 to 168 (f) and relative quantification (g). n=2 independent experiments.

FIG. 33: Flk1 marks a vascular-like compartment. a, b FAGS analysis of Flk1-GFP gastruloids from 96 to 168 (a) and relative quantification (b). n=2 independent experiments. c, FACS analysis of Gata6-Venus gastruloids showing co-expression of Flk1 and CD31. n=2 independent experiments. d, angiogenesis assay showing inability to form vascular-like tubes by undifferentiated mESCs and Flk1⁻ cells. Scale bar, 100 μm.

### EXAMPLES

### Example 1

### Multi-Axial Self-Organisation Properties of Mouse Embryonic Stem Cells and Induced Pluripotent Stem Cells into Gastruloids

The emergence of multiple axes is an essential element in the establishment of the mammalian body plan. This process takes place shortly after implantation of the embryo within the uterus and relies on the activity of Gene Regulatory Networks (GRNs) that coordinate transcription in space and time. While genetic approaches have revealed important aspects of these processes, a mechanistic understanding is hampered by the poor experimental accessibility of early post-implantation stages. Here we show that small aggregates of murine Embryonic Stem cells (ESC) stimulated to undergo gastrulation-like events and elongation in vitro, are capable of organising a post-occipital pattern of neural, mesodermal and endodermal derivatives that mimic the embryonic spatial and temporal gene expression. The establishment of the three major body axes in such ‘gastruloids’ suggests that the mechanisms involved are interdependent. Specifically, gastruloids display the hallmarks of axial gene regulatory systems as exemplified by the implementation of Hox collinear transcriptional patterns along an extending anterior-posterior axis. These results reveal an unanticipated self-organising capacity for aggregated ESC and suggest that gastruloids may be used as a complementary system to study early developmental events in the mammalian embryo.

Materials and Methods

ES/iPS cells and gastruloid cultures: The culture conditions and a detailed protocol for ES/iPS cells culturing and gastruloid production are provided below.

Animal experimentation: wild-type CD1 mouse embryos were used for RNAseq experiments. All experiments were performed in agreement with the Swiss law on animal protection (LPA) under license number GE 81/14 (to D. Duboule).

Libraries and qPCR analysis: Purified RNA from iPS cell derived gastruloids was retrotranscribed using the Promega GoScript retrotranscription kit. Quantitative PCR analysis of mRNA levels for different Hoxd genes, Bra and the housekeeping gene Hmbs was performed using the Syber select master mix for CFX (Thermofisher) kit according to manufacturer instruction and specific primers. The Biorad CFX96 thermocycler was used. At least two technical (PCR) replicates and two biological replicates were analyzed per time-point after aggregation.

Data availability statement. All RNAseq datasets produced in this study are publicly available in the Gene Expression Omnibus (GEO) database under #GSE10622.

Reagents & Equipment

Routine Culture Medium:

ESLIF Medium (1)
500 mL Glasgow's Minimal Essential Medium (GMEM, Gibco 11710-035),
5 mL sodium pyruvate (Invitrogen 11360-039),
5 mL non-essential amino acids (Gibco 11140-035),
5 mL GlutaMAX (Gibco 35050-038),
1 mL β-mercaptoethanol (Gibco 31350-010),
50 mL Foetal Bovine Serum (FBS, Biosera FB-1090/500),
550 μL Leukaemia Inhibitory Factor (1000 units, Merck Millipore ESG1107).

or

ESLIF Medium (2) (e.g. for culture of Sox1^{eGFP}; Bra^{mcherry} double reporter (SBR) mESC line, Oct4:GFP miPSC line)
500 mL Dulbecco's Modified Eagle's Medium (DMEM, Gibco 11960044),
5 mL sodium pyruvate (Invitrogen 11360-039),
5 mL non-essential amino acids (Gibco 11140-035);
5 mL GlutaMAX (Gibco 35050-038);
1 mL β-mercaptoethanol (Gibco 31350-010);
50 mL Foetal Bovine Serum (FBS, Biosera FB-1090/500),
550 μL Leukaemia Inhibitory Factor (1000 units, Merck Millipore ESG1107),
3 μM CHIR99021 (Tocris Biosciences 4423),
2 μM PD0325901 (Tocris Biosciences 4192).

Differentiation medium:
NDiff227 (Takara, Y40002).

or

N2B27
500 mL Neurobasal (Thermo Fisher Scientific, 21103-049),
500 mL DMEM-F12 (Sigma-Aldrich, D6421),
5 mL N2 (see below, or Merck Millipore SCM012),
5 mL B27 (Thermo Fisher Scientific, 17504-044),
10 mL glutamine (Thermo Fisher Scientific 25030081),
1 mL β-mercaptoethanol (Gibco 31350-010).

Reagents:
CHIR99021 (Chiron, 10 mM in dimethyl sulphoxide (DMSO), Tocris Biosciences 4423)
Gelatin (Sigma-Aldrich G1890); dissolved in distilled water to a 1% (w/v) stock solution, autoclaved and further diluted to 0.1% in 1×PBS (see below) prior to use.
1× Phosphate buffered saline (PBS, with Mg²⁺ and Ca²⁺; Sigma-Aldrich D8662).
Trypsin-EDTA (0.05%, Gibco25300-1010).

Plastics:
15 mL or 50 mL Centrifuge tubes (Grenier Bio-One 188271 or 227261)
25 cm² tissue culture flask (Grenier Bio-One 690-175).
Sterile reservoir (55 mL, STARLAB E2310-1010).
U-bottomed non-tissue culture-treated 96-well plate (Grenier 650185 or CLS 7007).
Optional: Low-adherence 24-well plate (Sigma Aldrich CLS3473) for extended culture.

Equipment:
BSL-2 biosafety cabinet.
Benchtop centrifuge for 15 mL/50 mL centrifuge tubes.
Haemocytometer (e.g. Improved Neubauer haemocytometer, Hawksley AS1000) or automated cell counter (e.g. TC20, Bio-Rad 1450102 or Moxi Z Mini, ORFLO Technologies MXZ002).
Humidified cell culture incubator (37° C., 5% CO₂).
Inverted benchtop microscope for examination of cultures.
Multichannel micropipette (30-300 μL is an ideal range).
Water bath (37° C.).
Optional: Incubator compatible orbital shaker (Infors Celltron 69222) for extended culture.

Procedure

Culture Conditions Prior to Aggregation:
Maintain mESCs in ESLIF medium (see Reagents & Equipment) on 0.1% gelatin-precoated tissue culture-treated plastic (e.g. 25 cm² flasks) in a humidified incubator (37° C., 5% CO₂).
Passage the mESCs to new flasks every other day; exchange 50-66% of the culture medium for fresh, pre-warmed medium on alternating days.
Culture the mESCs for at least two passages post-thawing before experimental use. Stocks that have been maintained in vitro for more than 30 passages may produce more variable results.
Culture cells to 40-60% confluence at the point of passaging or experimental use. A density of 1.8×10⁴ cells/cm² is recommended, but this should be optimised for each cell line.
Routinely test all cell lines for Mycoplasma contamination and maintain stocks under antibiotic free conditions to quickly identify microbial infections.

0 hours: Preparation of Gastruloids from mESCs and miPSCs (one 96-well plate)

Note 1: The following protocol describes generation of gastruloids from mESCs and miPSCs. Gastruloids can be reproducibly generated with mESC and miPSC lines from genetic backgrounds that include the 129 strain. The culture requirements may differ for mESCs and miPSCs (e.g. the use of ES+LIF Medium1 or 2, respectively). Such differences can also be observed between different mESC lines from different genetic backgrounds.

Note 2: Gastruloids derived from miPSCs generally require higher starting cell numbers (e.g. 600-800 cells/well) compared to mESC-derived gastruloids (e.g. 300 cells/well). The optimum starting cell number should be defined empirically.

Note 3: Gastruloids can be formed successfully from cells cultured in 2i conditions (N2B27+3 μM Chi+1 μM PD0325901+LIF), although the process of elongation is slightly delayed with respect to cells from ESL medium.
1. Pre-warm PBS, ESLIF, N2B27 and Trypsin-EDTA in a 37° C. waterbath.
a. Optional: If maintaining the cell stock, pre-coat a T25 tissue culture flask with 5 mL 0.1% Gelatin in PBS.
2. Aspirate the medium from the T25 tissue culture flask and rinse gently with 5 mL PBS, twice.
3. Aspirate the PBS and add 1 mL of pre-warmed Trypsin-EDTA.
4. Rock the flask to detach the colonies of cells. If required, strike the wall of the flask gently a few times to aid detachment, or incubate the flask at 37° C. for 30 seconds.
5. Dissociate the colonies into a single cell suspension with a P1000 micropipette tip by ejecting the suspension forcefully against a wall of the flask.
a. CRUCIAL: A single cell suspension is essential for accurate cell counting. Errors in counting affect the size of the gastruloids, which is known to affect the level of axial organisation.
6. Neutralise the Trypsin-EDTA with 5 mL ESLIF and transfer to a centrifuge tube.
7. Centrifuge the suspension for 3 minutes at approximately 170×g.
8. Aspirate the supernatant and add 5 mL warm PBS.
a. Tip: The pellet should become resuspended by the addition of the PBS. In order to avoid the loss of cells through transfer errors, drawing the suspension into the pipette is discouraged.
9. Centrifuge the suspension for 3 minutes at approximately 170×g. 10. Aspirate the supernatant and add 5 mL warm PBS.
11. Centrifuge the suspension for 3 minutes at approximately 170×g.
12. Aspirate the PBS, minimising carry-over by tilting the tube to remove as much as possible while leaving the pellet intact.
13. Fully resuspend the pellet in 1 mL N2B27 using a P1000 micropipette.
a. Optional: If required, dilute this suspension further in N2B27 to facilitate cell counting. Work with the newly diluted suspension for subsequent steps.
14. Load the haemocytometer (or the counting slide of an automated cell counter) with
10 μL of cell suspension and determine the density of the suspension.
15. Determine the volume of suspension required to produce a cell concentration of 7.5 mESCs/4 or 20 miPSCs/4 in N2B27.
E.g. 3.75×10⁴ mESCs or 10×10⁴ miPSCs in a final volume of 5 mL N2B27 is sufficient for a single 96-well plate, plus a small amount of dead volume; this gives 300 mESCs or 800 miPSCs per 40 μL drop after plating.
Tip: The number of cells per aggregate is adjusted empirically for each cell line to give aggregates of around 150 μm diameter at the 48 hour time point.
16. Add the calculated volume of suspension to the required amount of N2B27, mix well and transfer to a sterile reservoir.
a. Tip: Vortexing the suspension prior to plating or pipetting it up and down within the reservoir can ensure that the cells are well mixed.
17. Pipette 404 of plating suspension into each well of a sterile, U-bottomed non-tissue culture-treated 96-well plate with a multichannel micropipette.
a. Tip: Take care to position the droplets in the bottom of each well and not clinging to the walls, as U-shaped droplets are required for efficient aggregation.
18. Confirm that cells can be seen within each well using the inverted benchtop microscope.
19. Return the plate to the incubator for 48 hours.
a. Optional: If maintaining the cell stock, aspirate the Gelatin from the T25 and add 6 mL pre-warmed ESLIF medium. Calculate the required volume of the cell suspension for 4.5×10⁵ cells and add this to the ESLIF medium (there is a small carry-over of N2B27). 48 hours: Addition of Secondary Medium
20. Pre-warm N2B27 in the 37° C. waterbath.
21. Prepare secondary medium as a 3 μM Chiron solution in N2B27. 16 mL is ample for a single plate.
a. Tip: Using secondary media with different compositions will yield different results that are often apparent as changes in morphology.
22. Transfer 150 μL of secondary medium to each well using the multichannel micropipette and a sterile plastic reservoir.
23. Return the plate to the incubator for a further 24 hours. 72 hours: Removal of Secondary Medium
24. Pre-warm N2B27 in the 37° C. waterbath.
25. Carefully remove 150 μL of the secondary medium from each well using the multichannel micropipette, holding it at an angle to aspirate slowly from the side of each well.
26. Add 150 μL of fresh, pre-warmed N2B27 to each well using the multichannel micropipette.
a. Tip: Add the fresh medium with sufficient force to move the gastruloids within the well, thereby avoiding adhesion to the plastic.
27. Return the plate to the incubator for a further 24 hours. 96 hours: Change of Medium
28. Repeat steps 24-27 to exchange 150 μL culture medium for fresh, pre-warmed N2B27. 120 hours: Change of Medium
29. If maintaining the culture, repeat steps 24-27 to exchange 150 μL culture medium for fresh, pre-warmed N2B27.
Extended Culture (120-168 h):
30. To prolong the culture, transfer the gastruloids individually into low-attachment 24-well plates in 700 μL volumes of fresh N2B27 at 120 hours.
Tip: Cut a P1000 micropipette tip approximately 5 mm from the end and use this to collect and transfer the gastruloids with minimal damage.
31. Incubate on an incubator-compatible orbital shaker for 48 hours at 40 RPM.
32. Replenish the medium after 24 hours (144 hours) by exchanging 400 μL medium for fresh N2B27.

Troubleshooting

Aggregation Failure.

Aggregation failure might originate from the U-bottomed 96-well plate of choice. Make sure to use the aforementioned plates for efficient aggregation. The culture is also sensitive to the starting state of the cells. Failure to aggregate has been observed in stocks that have been maintained at high confluence (>90%) or under stress (e.g. from missing daily medium changes or pH<6.5), resulting in the formation of large embryoid bodies but not gastruloids.

The cells aggregate, but the gastruloids disintegrate during the culture.

This is often observed as a progressive decompaction of the cells, starting at the edge of the tissue and proceeding inwards. A possible cause is batch-to-batch variability in the N2B27 medium, which needs to be prepared carefully and checked for the presence of any precipitates before use. When using commercially available N2B27, follow the manufacturer's protocol for storage and thawing to prevent precipitation. Other causes could be environmental; start by confirming that the CO₂ concentration and temperature are stable within the incubator.

Gastruloids fail to form, or disintegrate during culture.

The droplet volumes are initially small (40 μL) and so the plates are sensitive to evaporation during the first 48 hours. Check that the incubator is adequately humidified and, where possible, avoid areas of rapid air circulation (e.g. close to the fan, if present). This problem becomes evident as a reduction in droplet volume and changes in pH in the peripheral wells.

Many small satellite aggregates form, or the gastruloids are very small. These observations indicate problems in cell counting. In the former case, underestimation of the true cell density results in too many cells being plated in each well. This can produce many small satellite aggregates that can fuse with the main gastruloid, producing spurious elongated morphologies. Ensure that the suspension is fully dissociated to single cells before counting and that the plating suspension is well-mixed prior to use. Satellite aggregates may also form in sub-optimal U-bottomed 96-well plates. Make sure to use mentioned plates for efficient aggregation.

The gastruloids adhere to the plastic and lose their shape.

This becomes a common problem if the gastruloids are maintained beyond 120 hours in the non-tissue culture treated U-bottomed 96-well plates. It can be alleviated by adding the culture media with some force to move the gastruloids within the wells, or by using low-attachment 96-well plates. The extended culture protocol described above is recommended as a means of maintaining the gastruloids to 168 hours.

Time Taken

The total duration is 5-7 days, depending on whether the culture is extended. The hands-on time breaks down as follows:

0 hours: Approx. 30-45 minutes per cell line.

48 hours: Approx. 10 minutes per cell line.

72, 96 and 120 hours: Approx. 15 minutes per cell line.

120+ hours: Approx. 30 minutes per cell line to transfer the gastruloids to 24-well plates.

Anticipated Results

A brief overview of gastruloid development is detailed below:

Within the first 24 hours, the cell suspension sediments to the bottom of the wells, forming a single cellular aggregate in each well. Individual cells within the aggregates should be indistinct, indicating that they are fully adherent to their neighbours.

At the 48 hour time point, the aggregates should be smooth spheroids that have increased slightly in size over the preceding 24 hours.

At the 72 hour time point, the aggregates should have grown further, with some of the population remaining as spheroids and others showing regions of local narrowing, giving an ovoid appearance. The surface should no longer be smooth, with loose extruded cells forming a rough, but thin, coating on the tissue.

At the 96 hour time point, many of the aggregates will have proceeded from a spheroid or ovoid shape to become elongated along a clear long axis. One end of this axis should have a smooth border and appear bright under phase contrast microscopy, which corresponds to the elongating posterior end. The other end of the axis should be round and dark and be covered in a loose layer of extruded cells; corresponding to the more anterior tissue.

The optimal time to observe the elongations appears to be around 114-120 hours, by which point they should appear as long extensions from the darker, round-shaped anterior tissues. At later time points, some of the aggregates may start to adhere to the bottom surface of the wells and the tissue organisation will be disrupted, unless the extended culture technique is used.

Extended Cultures

In these cases, shaking the gastruloids in a larger volume extends the length of the elongations, allowing them to form very long and thin tissues. It is also be possible to observe internal epithelial tissues by phase contrast microscopy.

Results

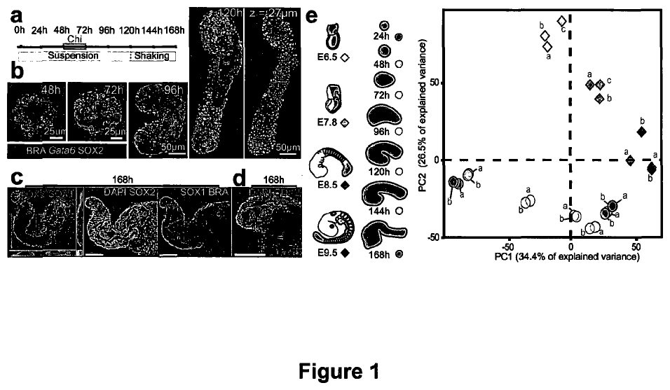

When ca. 250 ESCs are aggregated, given a pulse of the Wnt agonist CHIR99021 (Chi) between 48 and 72 h of culture, and returned to N2B27 medium (FIG. 1a), a pole of T/Brachyury (Bra) expression, resembling the elongating embryonic tail bud emerges reproducibly (FIG. 1b, FIG. 5). The aggregates keep elongating up to 120 h after aggregation (AA), when they display a ‘rostral’ cell-dense region and a polar extension towards a ‘caudal’ extremity reaching up to 500 μm in size (FIG. 1b). Shaking the culture allows to reach 850-1000 micron in length at 168 hrs AA (FIG. 1c,d). At these late stages, a Gata6-positive domain is detected at the opposite side of a Bra and Cdx2 expressing region, likely corresponding to the cardiac crescent, which delimits the embryonic post-occipital region (FIG. 1b-d, FIG. 5). In contrast, Sox1/Sox2 positive cells localised centrally, with the exception of the rostral-most portion (FIG. 1c, d).

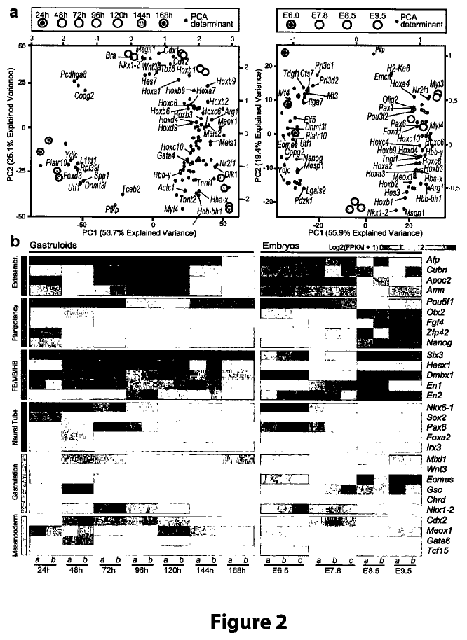

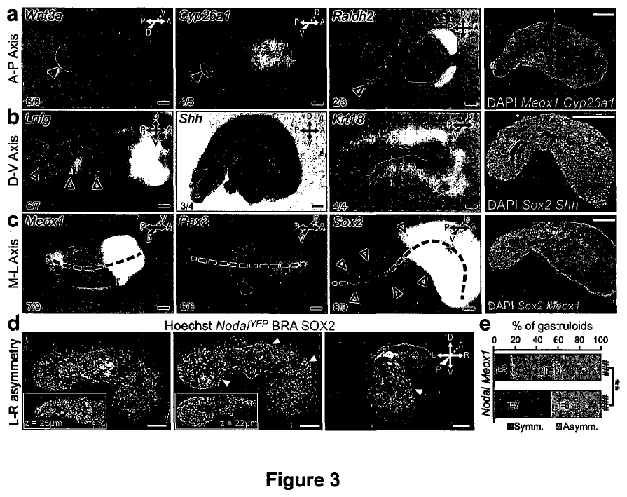

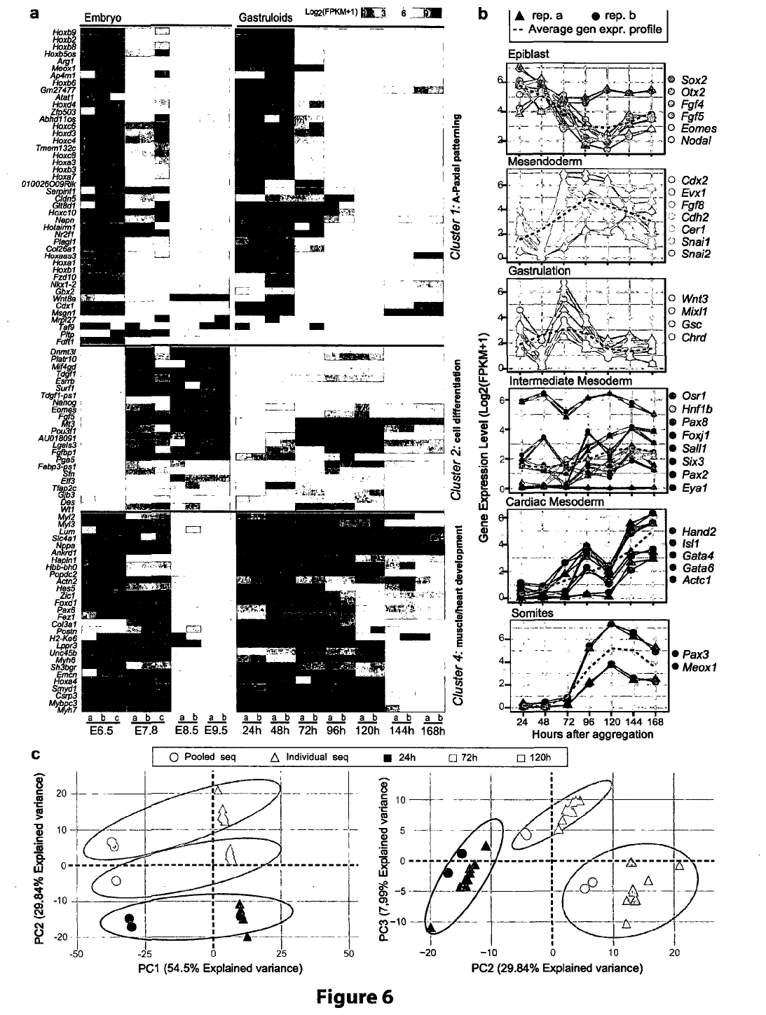

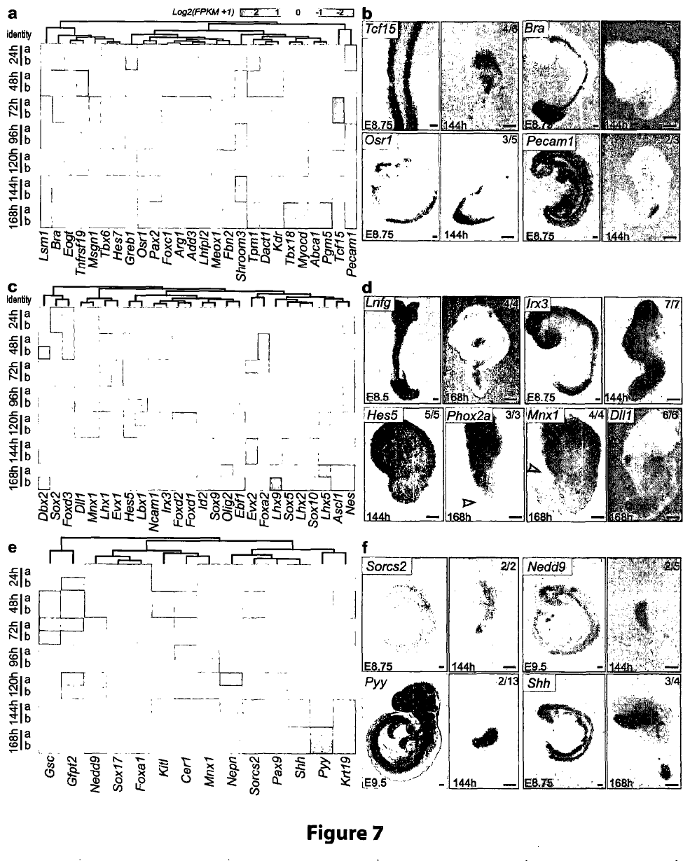

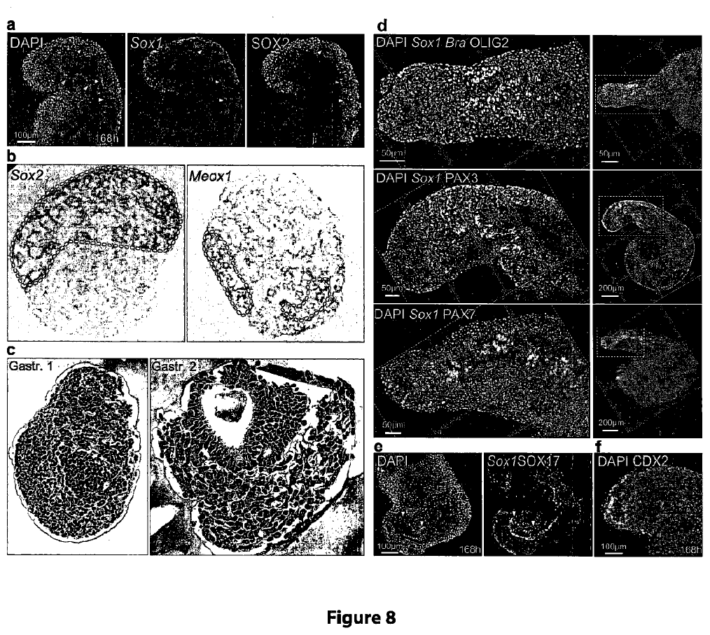

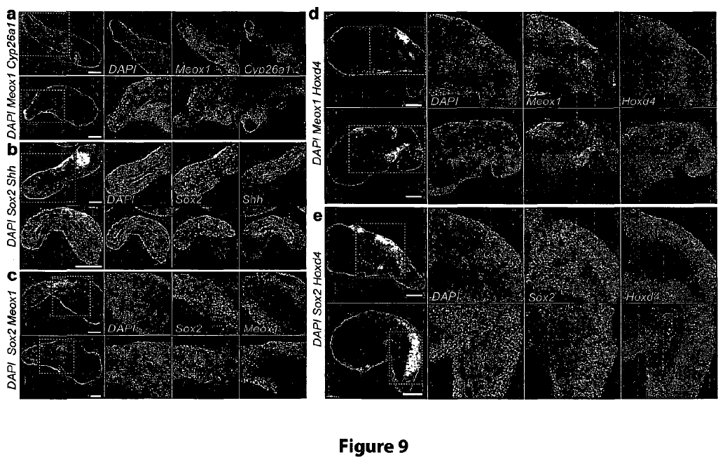

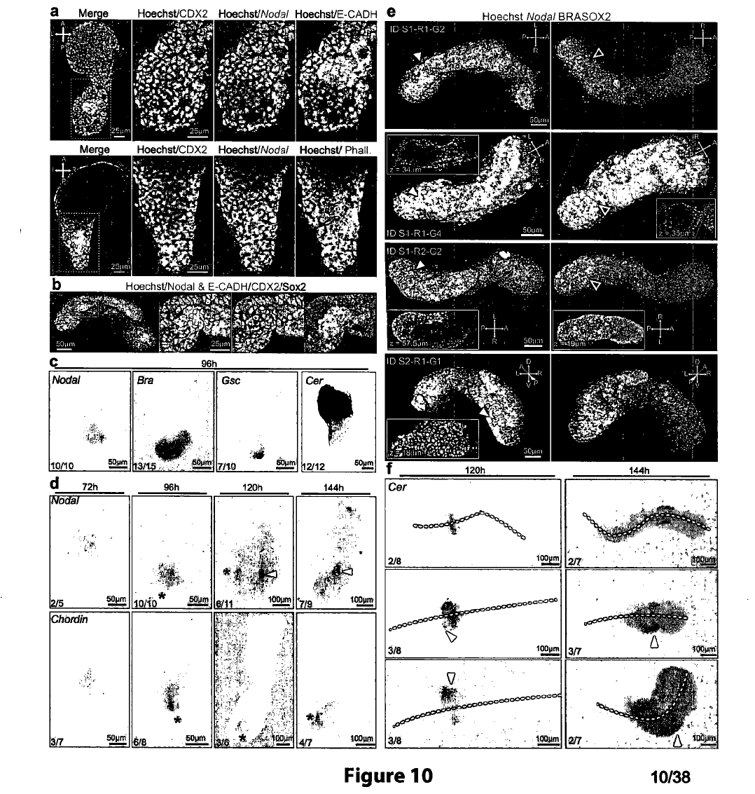

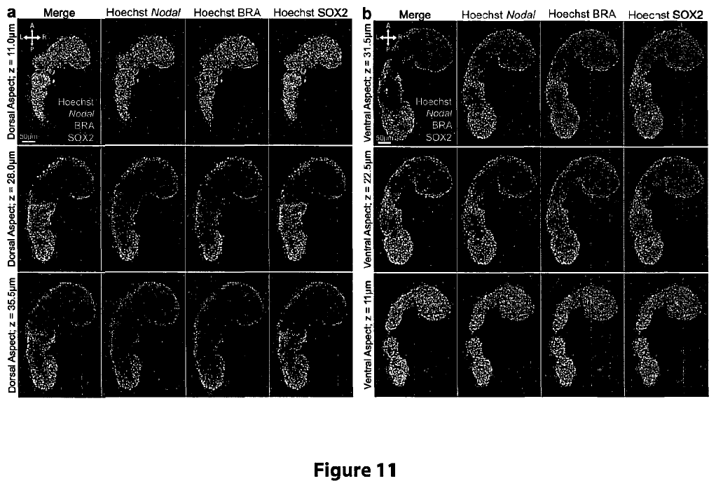

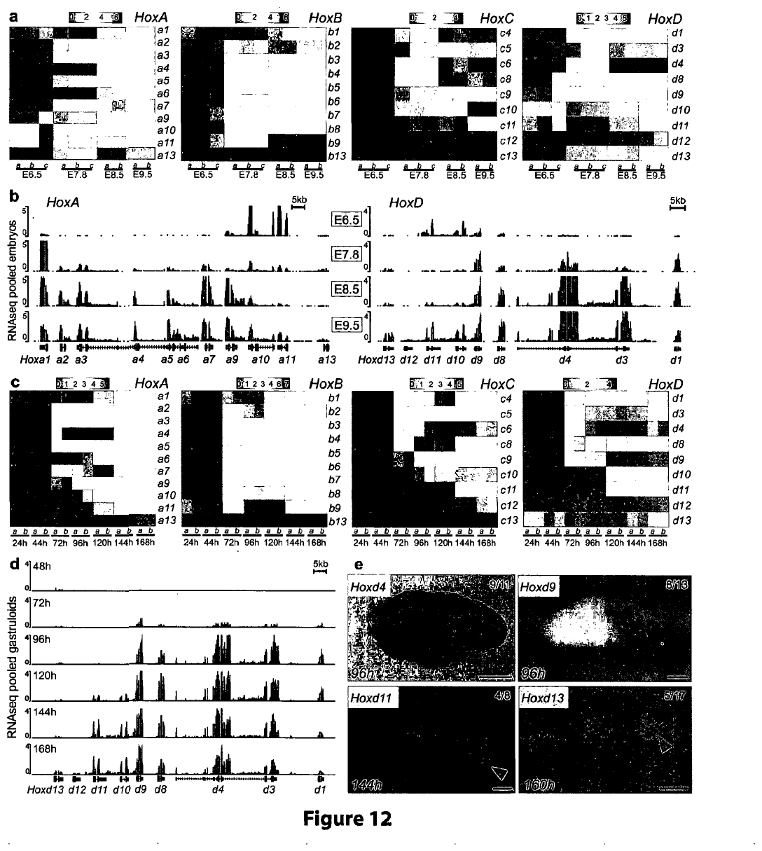

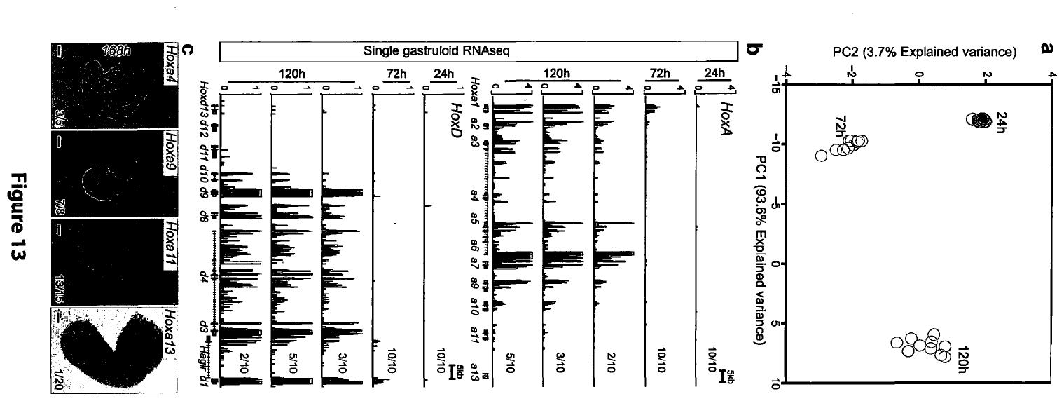

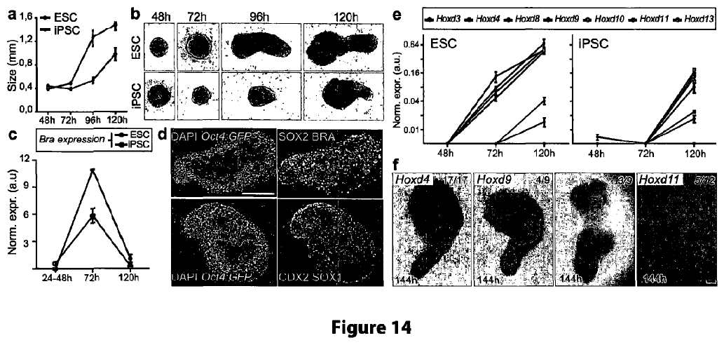

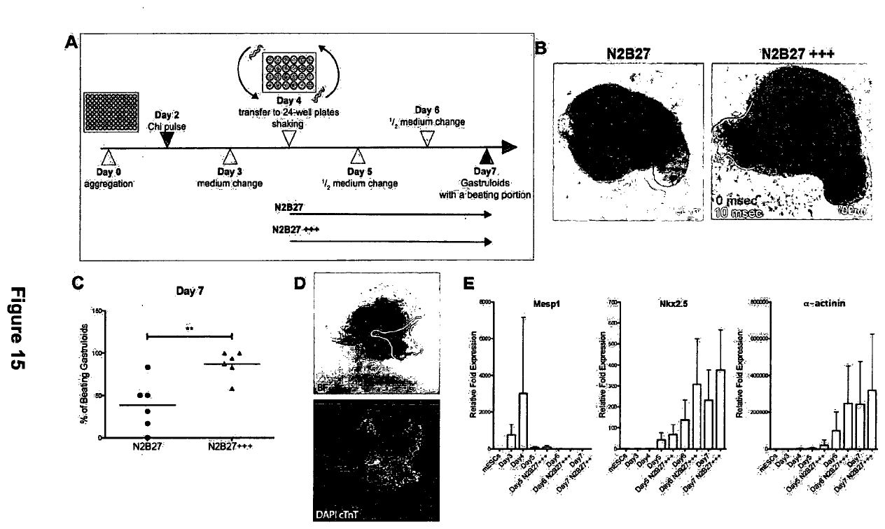

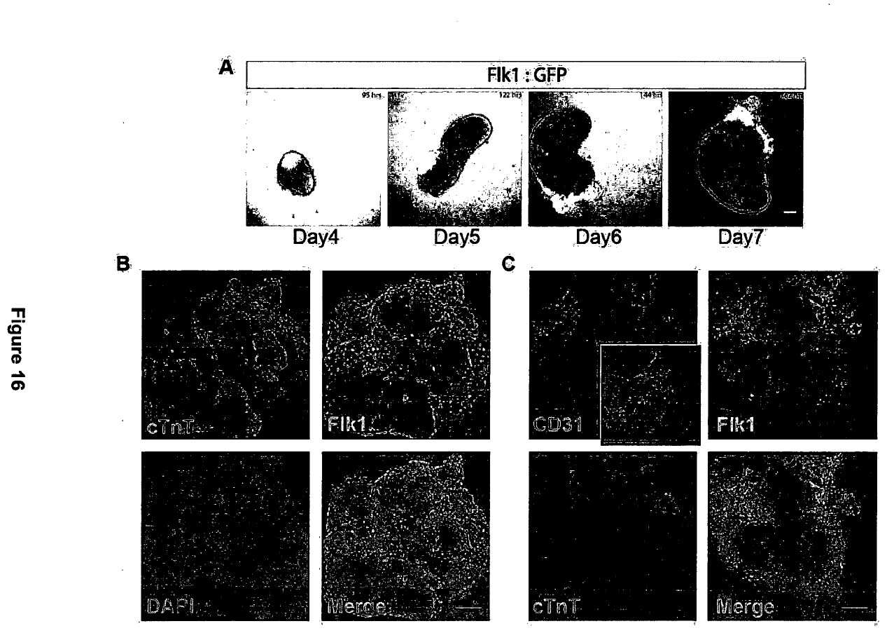

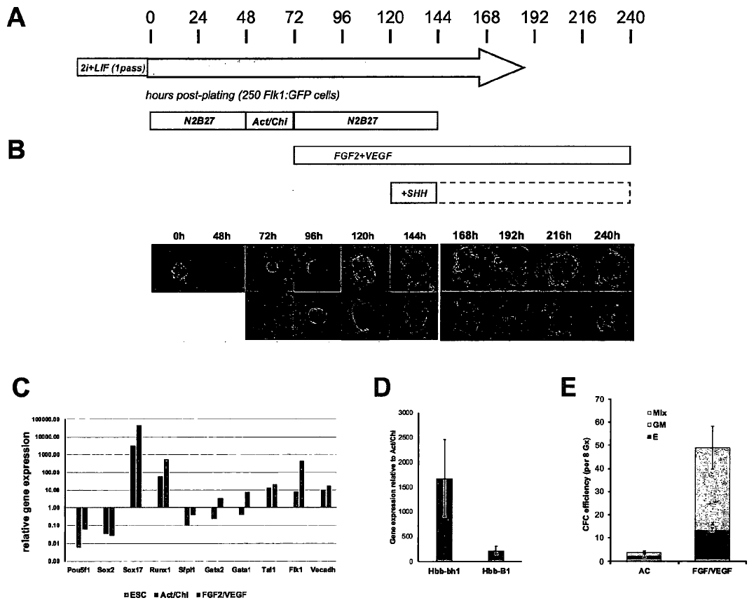

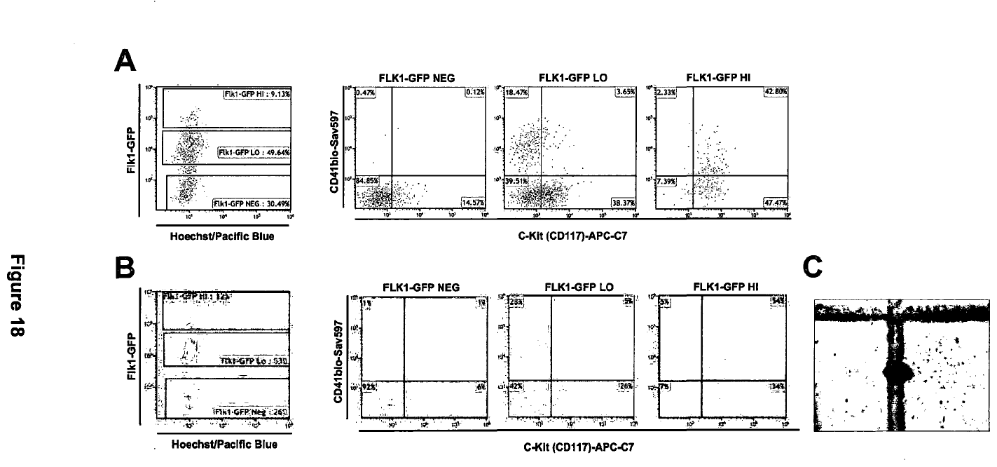

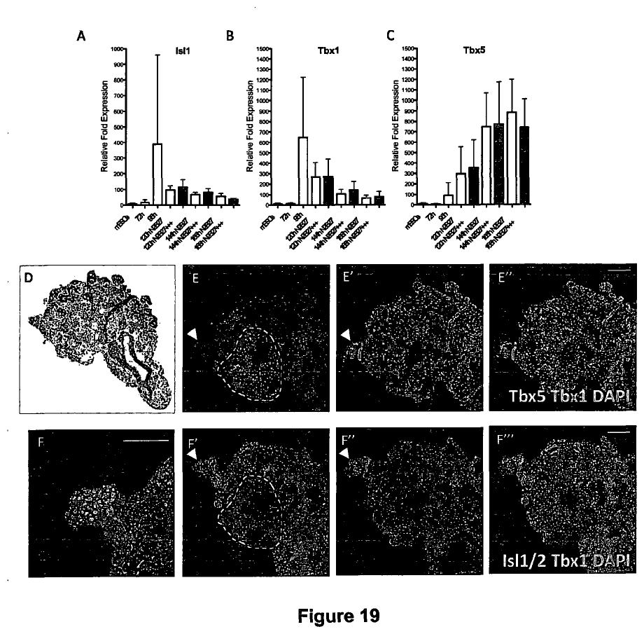

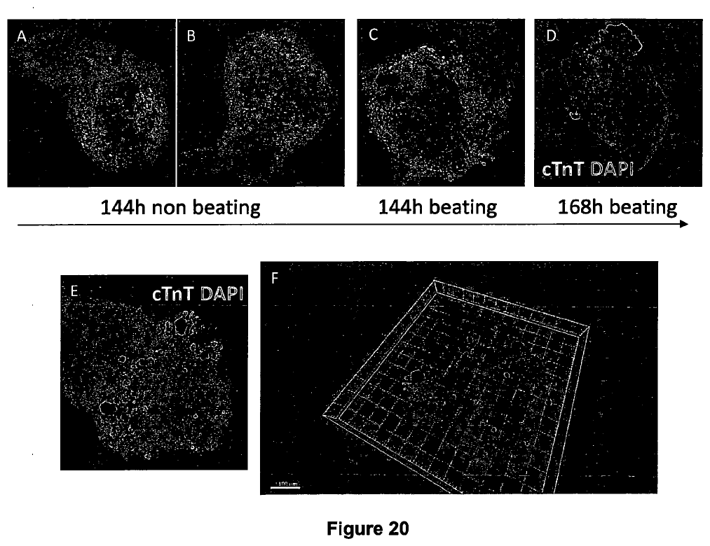

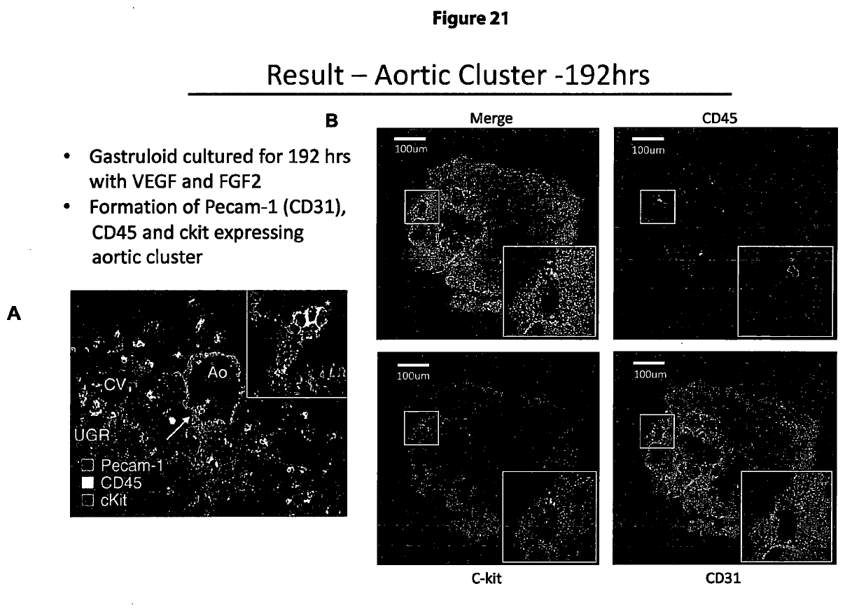

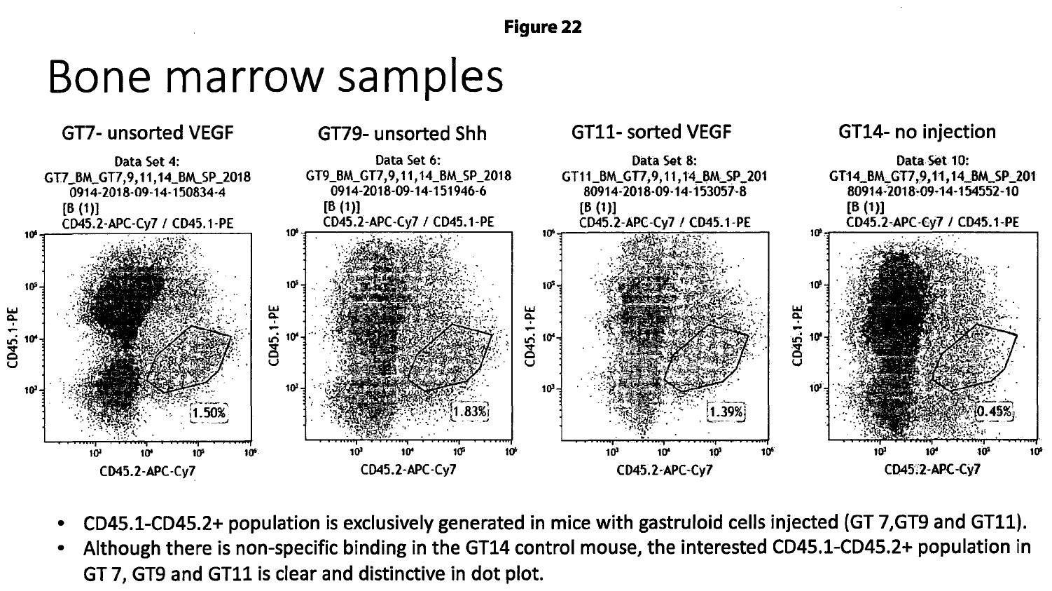

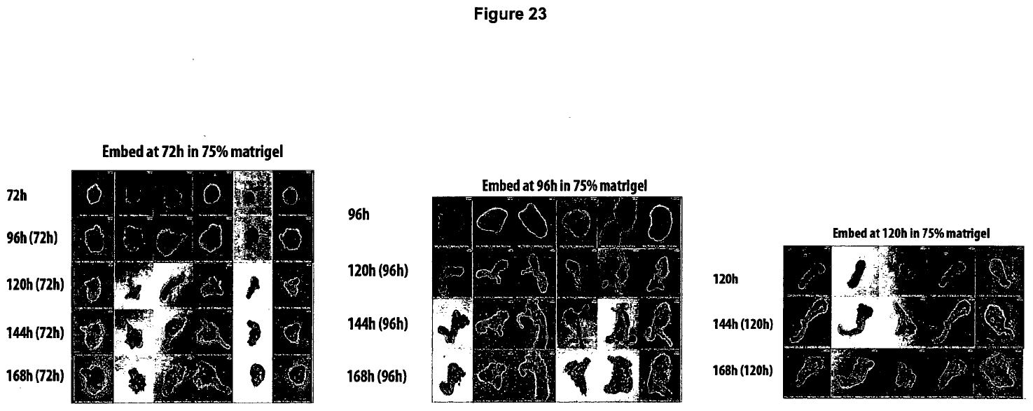

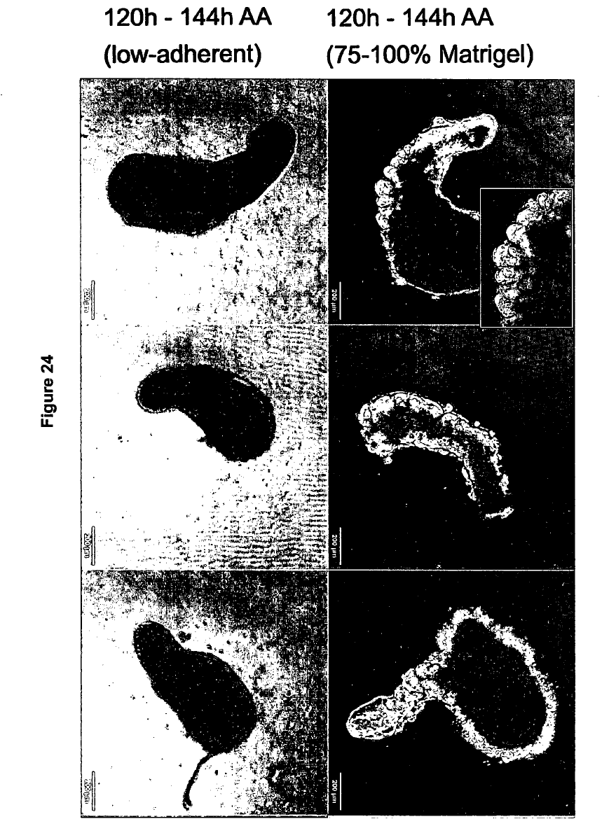

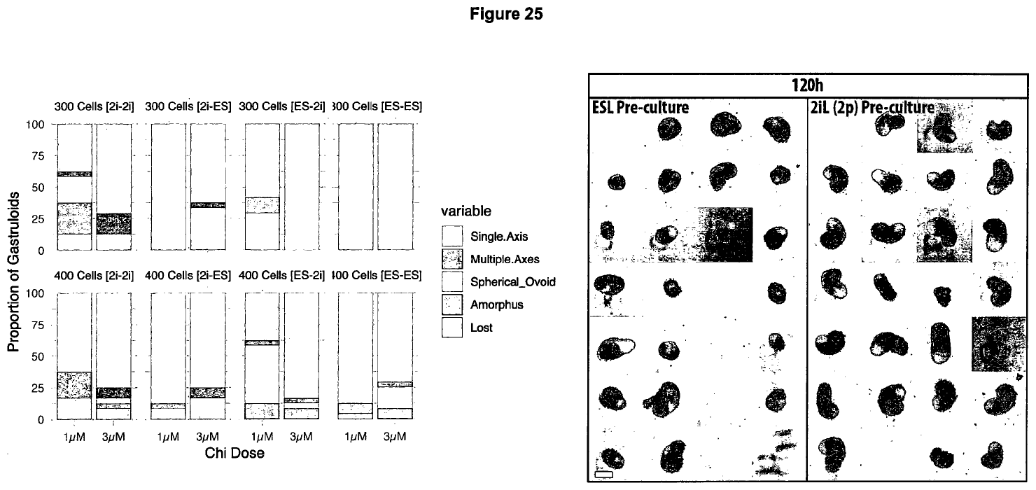

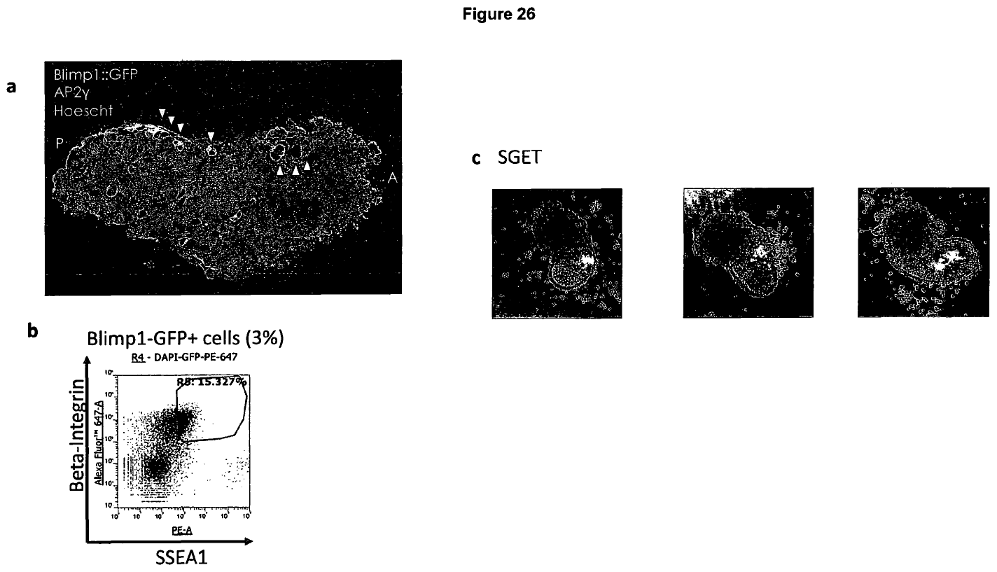

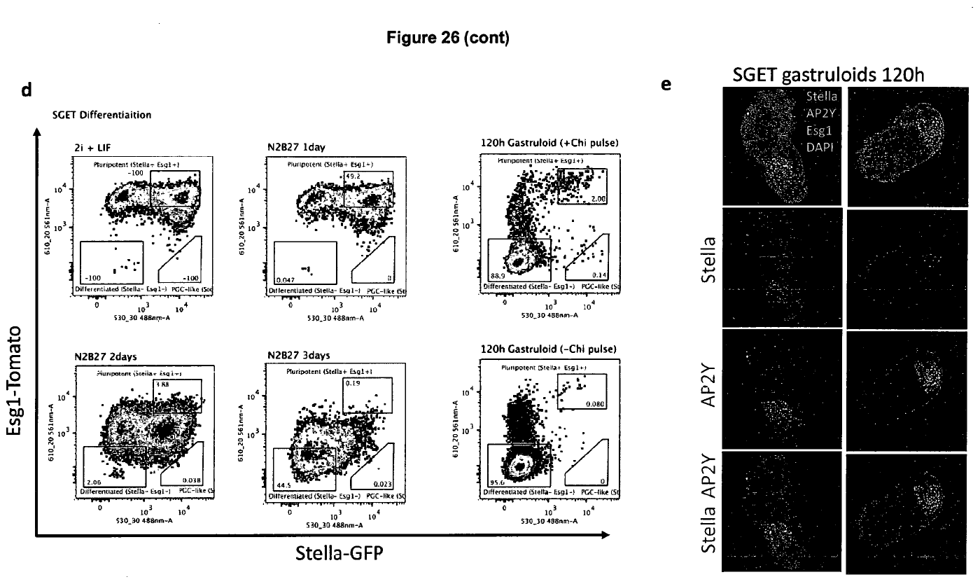

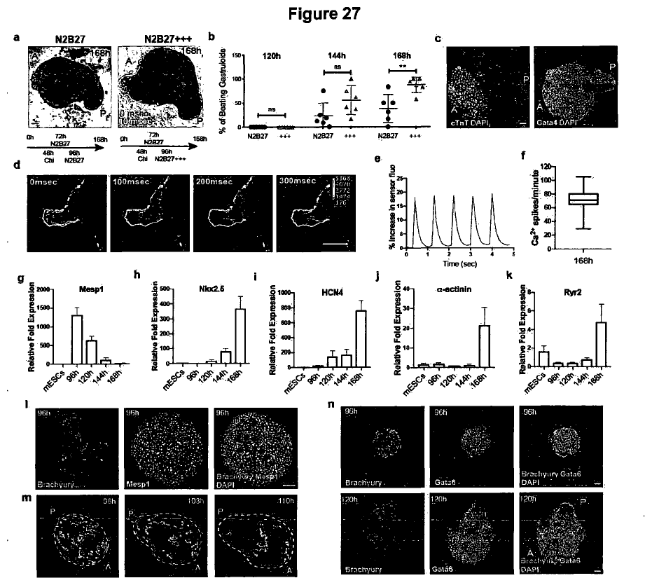

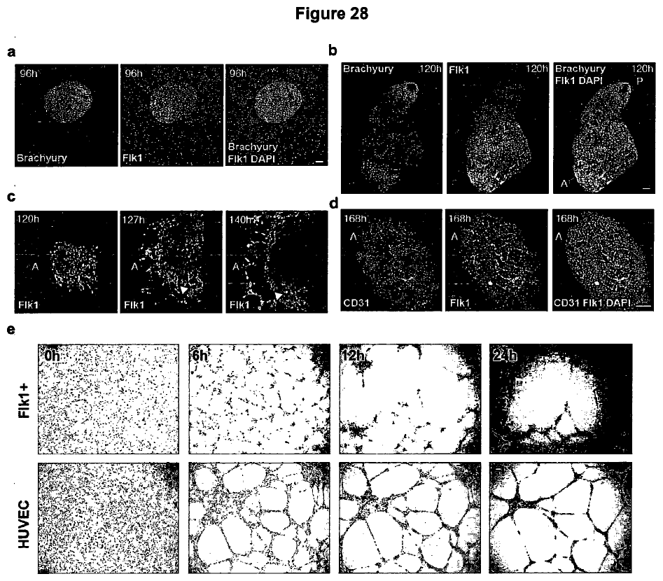

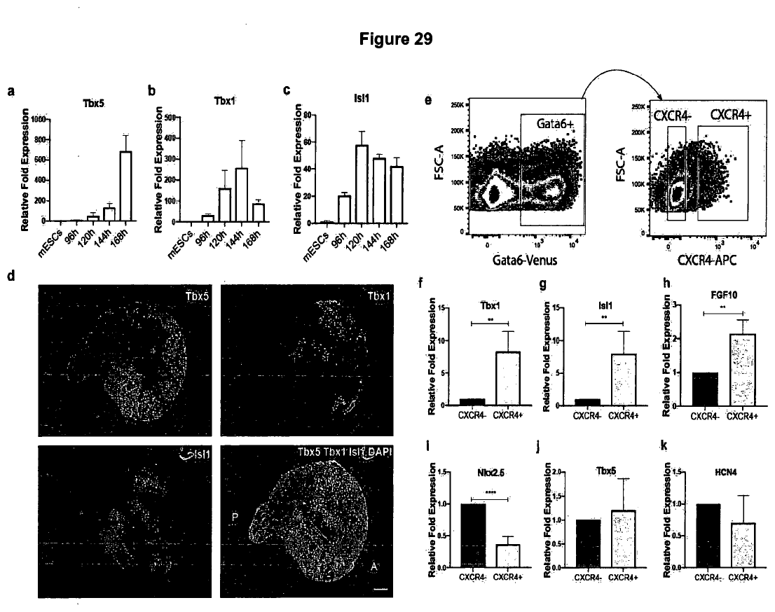

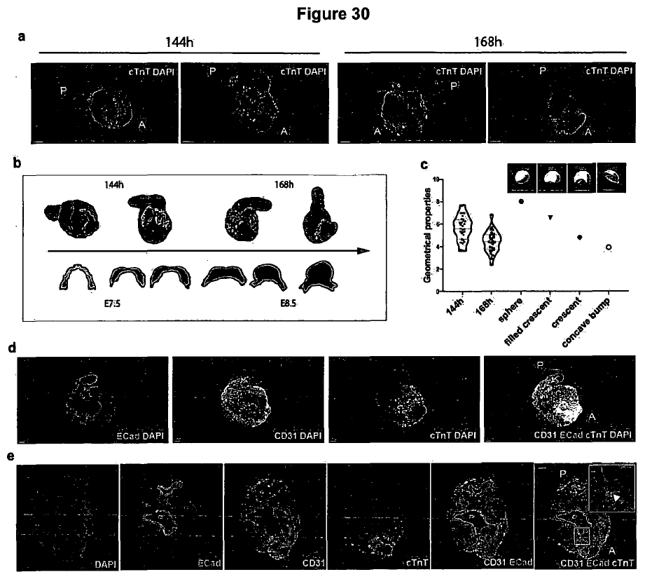

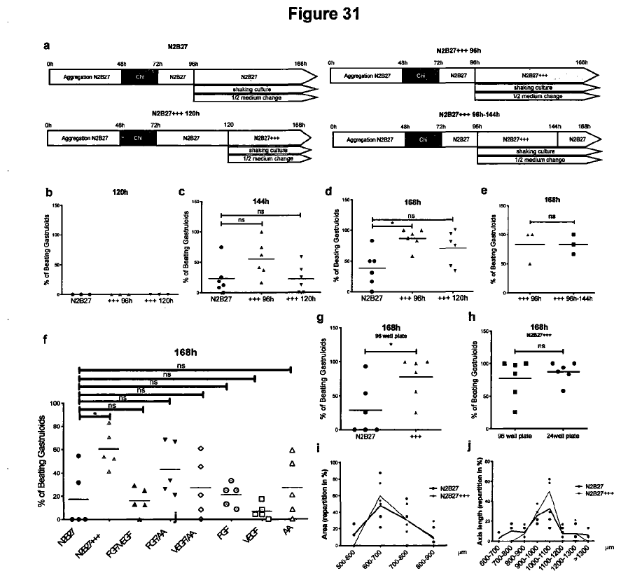

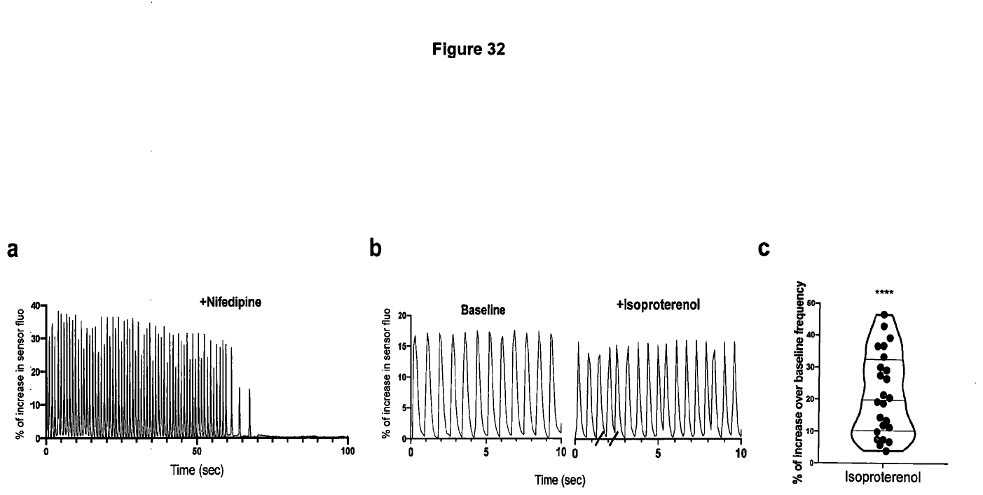

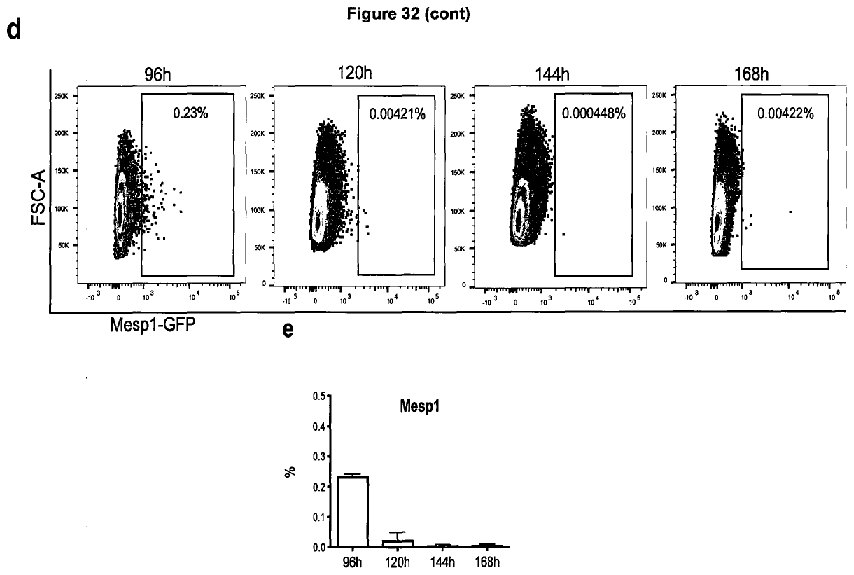

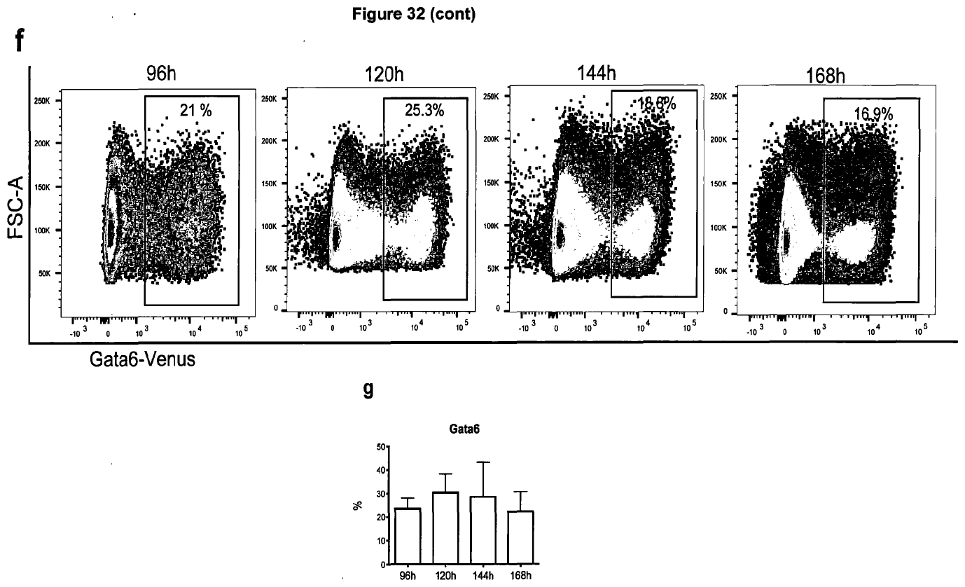

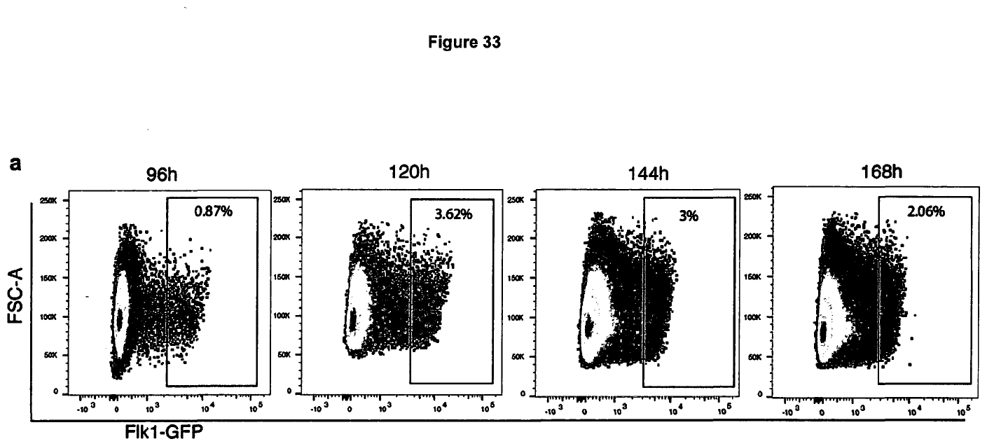

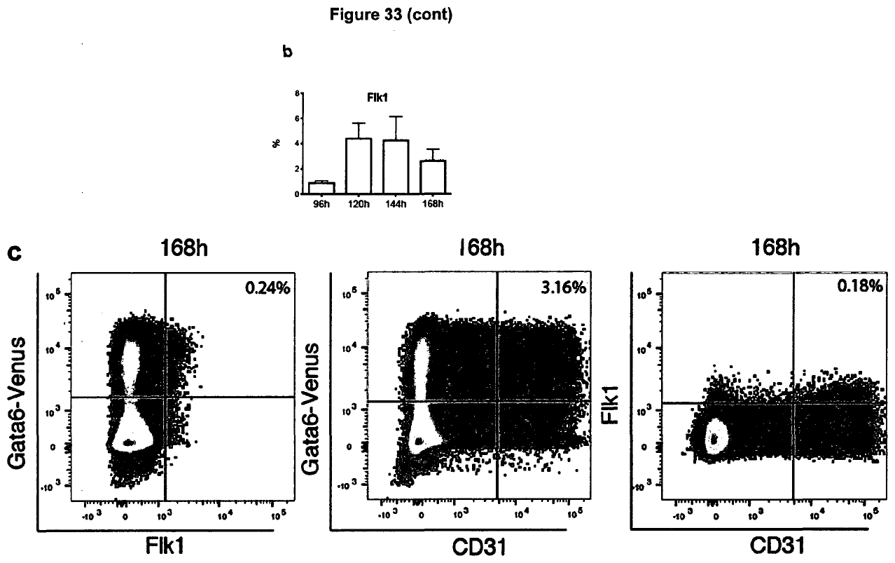

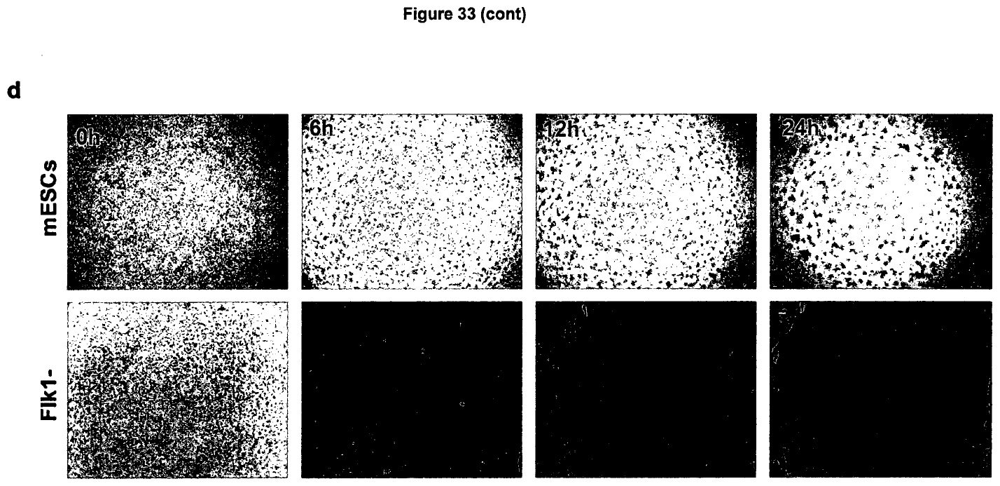

To characterize the transcriptional programmes of these gastruloids, we carried out RNAseq on duplicated pools and compared their profiles with those of developing mouse embryos from E6.5 to E9.5. Since gastruloids display hallmarks of post-occipital embryos (FIG. 1b-d) we excluded the anterior portion of E7.5-E9.5 embryos (FIG. 1e, top). Principal Component Analysis (PCA) showed reproducibility between samples and a clear clustering along PC1 corresponding to the temporal order of samples (FIG. 1e), while embryo samples segregated from gastruloids in PC2 only. The main (top 100) clustering determinants of gastruloid samples included several pluripotency-related genes, epiblast markers and genes involved in gastrulation, as well as Hox genes and other transcription factors such as Cdx1/2, Meis1/2, Meox1, Bra, and Gata4 (FIG. 2a). These genes are normally expressed in post-occipital structures of the developing mouse embryo. 25 out of 100 of these PCA determinants were identified independently in both gastruloids and embryos temporal series, (FIG. 2a, red-labelled genes) supporting the idea that gastruloids and embryos elongate by implementing similar transcriptional programs. The analysis of specific genes associated with particular developmental landmarks confirmed this point (FIG. 2b, FIG. 6b). For instance genes associated with gastrulation like Mix11, Eomes, Gsc or Chrd were transiently and orderly transcribed at around 48 h AA (FIG. 2b and FIG. 6), suggesting that at this stage the gastruloids transcriptome resembles that of mouse epiblast at the onset of gastrulation. By 72 h AA, we observed an increase in the complexity of gene expression profiles, with the appearance of markers for different embryonic lineages including mesendoderm and neuroectoderm and the transcription of Hox gene clusters (FIG. 6 and FIG. 2a, b; see below). Genes associated either with extraembryonic structures or with anterior neural plate derivatives were not (or poorly) expressed in gastruloids (FIG. 2b). PCA analysis using single gastruloids revealed a robust clustering for any developmental stage assessed and with the pooled RNAseq datasets (FIG. 6c) showing that the population of gastruloids was rather homogenous at the time points analysed and hence that the pooled RNAseq datasets reflected the transcription status of single gastruloids. Gastruloids transcriptome analyses revealed mRNAs normally associated with neural, endodermal and mesodermal derivatives, including paraxial, cardiac, intermediate and hematopoietic progenitors as well as neural crest (e.g.^{8,9}) (FIG. 2b; FIGS. 6b and 7). We also observed an antero-posterior pattern of differentiation along these lineages, reminiscent to what occurs in the embryo. For example, the sequential expression of Bra, Msgn1, Meox1/Tcf15 recapitulates the spatio-temporal differentiation pattern of paraxial mesoderm (FIG. 7a, b). In neural tissue, while Hes5 and Dll1 were strongly expressed during gastruloid extension, the density of terminally differentiating Phox2a/Mnx1 positive cells formed an apparent anterior to posterior gradient, almost completely absent from the posterior-most aspect (FIG. 7c, d). These ordered patterns of gene expression nevertheless did not correlate with any precise morphogenesis. Neural markers (Sox1, Sox2, Lnfg) were expressed in a continuous domain, yet without forming a proper neural tube-like structure (FIGS. 7d and 8a-c; FIG. 3) even though sporadic tubular structures were scored along this domain (FIG. 8a, white arrowheads, c, right panel). Also, clumps of cells positive for either SOX1 and OLIG2, SOX1 and PAX3 or SOX1 and PAX7, indicative of dorsal and ventral neural tube progenitors, were observed (FIG. 8d), yet without a clear segregation along the dorso-ventral extension of the SOX1 domain. Similarly, Tcf15 expressing cells did not condense into somites (FIG. 7b).

The analysis of different endodermal markers revealed temporal dynamics also reminiscent of the embryonic situation¹⁰ (FIGS. 7e, f and 8e-f). Gsc and Cdx2 transcripts, markers of definitive endoderm, were upregulated soon (72 h AA) after Chiron induction, followed by Cer1 (96-120 h AA) and by Sorcs2, Pax9 or Shh subsequently (120 h-144 h AA). All endoderm expressed genes assayed (Sox17, Sorcs2, Nedd9, Pyy, Cdx2 and Shh) were active in the ventral-like domain of gastruloids (FIG. 3, FIGS. 7f, 8e-f), resembling the embryo situation. Of note, Cdx2 transcripts were confined to the posterior most gastruloid endoderm (FIG. 1d, FIG. 8f), in agreement with this gene specifying the hindgut domain. In a majority of cases, gut-endoderm progenitors appeared as a continuous tubular structure (FIG. 8a, e-f; red arrowheads), often spanning the entire antero-posterior extension, reminiscent of an embryonic digestive tract.

This unanticipated level of organization and capacity to self-organize an integrated axial system reminiscent of the embryo was further explored by assessing the expression of genes associated with the developing embryonic axes (FIG. 3). Wnt3a and Cyp26a1 transcripts were scored at the caudal extremity of gastruloids similar to Bra and Cdx2 (FIG. 1c, d; FIGS. 7a, d and 8a, d, FIG. 9a), complementary to the localization of Raldh2 mRNAs further supporting the existence of an antero-posterior axis. On the other hand, Lnfg, Sox1 and Sox2 were transcribed in a central and dorsal domain at 144 h AA (FIGS. 1b, 3b and FIGS. 7a and 8a), complementary to the ventrally located intestinal tract markers (FIGS. 3b, FIGS. 7d and 8c, d, FIG. 9b). Additional signs of multi-axial organisation were provided by the expression of mesoderm specific genes Osr1, Pecam, Meox1 and Pax2 in a medio-lateral symmetry flanking the centrally located Sox2 positive domain (FIG. 3c). Double staining of Sox2 and Meox1 (FIG. 3c, FIG. 9c) and cross-sections (FIG. 8b) confirmed the medio-lateral and dorso-ventral distribution of neural and mesodermal progenitors.

Nodal expression was found confined to a small and compact region on the ventral most posterior aspect at 120 h AA (FIGS. 10 and 11). These cells displayed high levels of E-cadherin and a dense phalloidin staining (FIG. 10a, b) suggestive of a node-like identity, a hypothesis supported by the presence of Nodal mRNAs in a domain comparable to that of Goosecoid, Bra and Chordin at 96 h AA (FIG. 10c,d). Time course analysis of Chrd and Nodal indicated that such putative node-like cells were detected at 96 h and persisted until 144 h at least (FIG. 10d). Nodal mRNAs in these cells nevertheless rapidly decreased and were almost undetectable at 144 h AA. Despite these evidences for a node-like structure, we did not observe any notochord derivatives, which normally originate from the node, raising questions as to whether this putative structure may excert an organizing activity reminiscent to that of its embryonic counterpart. The downregulation of Nodal in presumptive node-like cells at 120 h AA coincided with patches of Nodal-expressing cells along the posterior half of extending gastruloids, often distributed in an asymmetric manner (FIG. 3d, FIG. 10d, e). This pattern was maintained at 144 h AA (FIG. 10d) but was not observed with Meox1, which was predominantly expressed on both sides (FIG. 3d, e). Accordingly, the Nodal target gene Cerberus was also expressed asymmetrically at both 120 h AA and 144 h AA (FIG. 10f). Altogether these data suggest that besides the establishment of a medio-lateral axis, gastruloids may implement the start of a left-right asymmetry,

The formation and patterning of post-occipital embryonic territories is tightly linked to the sequential activation of the 39 Hox gene, which are clustered at four distinct genomic loci in mammals. As Hox genes appeared differentially regulated in the RNAseq time-course (FIG. 2a, b, FIG. 6a), we assessed whether their sequential activation in time and space was recapitulated too. A pooled PCA analysis considering exclusively Hox genes transcripts, revealed robust clustering along the time axis (81% variance) and a close correspondence with the dynamic activation of these genes in embryo (FIG. 4a, FIG. 12a-c). The variability in Hox RNAs content amongst gastruloids was then evaluated using ten individual specimens from three different stages (FIG. 13a). Gastruloids from the same time-point tightly clustered together solely based on their Hox transcripts. Transcript profiles over Hox clusters revealed signs of collinear activation, the hallmark of this gene family. In E6.5 embryo, some Hoxa and Hoxd genes are expressed before gastrulation in extraembryonic tissues (FIG. 12a). From E7.8 to E9.5, Hox genes start to be transcribed in an order which reflect their 3′ to 5′ position within each cluster (FIG. 12a, b). The RNAseq profiling revealed an activation dynamic comparable to that observed in embryo (FIG. 2a and FIG. 12c). For instance while Hoxa RNAs were not detected until 48 h AA, Hoxa1 to Hoxa3 expressions were robust at 72 h, followed by sustained transcription of Hoxa5, Hoxa7 and Hoxa9 at 96 h to 120 h. Hoxa10 and Hoxa11 RNAs appeared at 144 h AA, at the same time Hoxa1, Hoxa2 and Hoxa3 transcripts started to disappear (FIG. 4b, FIG. 12c).

Similar dynamics were observed for Hoxd genes (FIG. 12c), which were activated in a sequence starting from 72 h AA until 168 h AA (FIG. 12c-e). The early transcription of 5′ Hoxa/Hoxd genes (FIG. 12a-b) was not observed in gastruloids (FIG. 13 and FIG. 4b), in agreement with the absence of extraembryonic derivatives.

Comparable profiles were also scored when single organoids were examined (FIG. 13a, b), again revealing the high reproducibility of this activation process. In the embryo this temporal activation is paralleled by a collinear distribution of transcripts in space. Likewise, Hoxa4/Hoxd4 displayed an AP boundary close to the anterior aspect of the gastruloid, whereas Hoxa9/Hoxd9, Hoxa11/Hoxd11 and Hoxd13 showed successively more posterior boundaries (FIG. 4c, FIG. 13c). Notably, Hoxd13 transcripts appeared in a population of cells located centrally at the posterior extremity, resembling its normal expression in the embryonic cloacal area (FIG. 4c). Hoxa13 expression was also detected at 168 h AA in the posterior aspect, yet rarely (1/20), in agreement with the low transcript levels detected in the pooled RNAseq analysis (FIG. 13c). Double staining for Hoxd4 and either Sox2 or Meox1 revealed expression of Hox genes in both neural and mesodermal derivatives (FIG. 4d, FIG. 9d, e). The implementation in space and time of the Hox gene network confirmed the surprisingly high level of organisation in the processing of gene regulatory networks, in particular without any extraembryonic component. We tested the ability of several induced pluripotent stem cells (iPSC) lines to produce gastruloids (FIG. 14) and a similar elongation process was observed in one of them. iPSCs can thus generate gastruloids. However, these gastruloids showed a reduced elongation rate, particularly between 48 h and 96 h (FIG. 14a, b). The expression dynamics of Bra in these iPSC gastruloids was nevertheless similar to their ESC counterparts. The neural markers Sox1 and Sox2 as well as Cdx2 were also expressed as in ESC-derived gastruloids (FIG. 14d, compare with FIG. 1b, c). Furthermore, iPSC gastruloids implemented Hoxd temporal and spatial collinear expressions, though with a delay in the expression onset and a spatial collinearity not as clearly organized as in ESC-derived gastruloids.

When compared to single tissue organoids, gastruloids exhibit an integrated structure, which seems to specify all major embryonic axes in a coordinated manner. The remarkable autonomy in the patterns of gene expression reported here highlights the potential of gastruloids in the study of complex regulatory circuits, particularly during early post-implantation development and the emergence of body axes.

### Example 2

### Organogenesis in Gastruloids

Materials and Reagents

25 cm² cell culture flask (Grenier Bio-One 690-175).

Activin A (10 mM, use at 100 ng/mL; Peprotech 120-14B). CHIR99021 (Chiron, 10 mM, Wellcome Trust-Medical Research Council Cambridge Stem Cell Institute).

ESLIF medium: 500 mL Glasgow's Minimal Essential Medium (GMEM, Gibco 11710-035); 5 mL sodium pyruvate (Invitrogen 11360-039); 5 mL non-essential amino acids (Gibco 11140-035); 5 mL GlutaMAX (Gibco 35050-038); 1 mL R-mercaptoethanol (Gibco 31350-010); 50 mL Foetal Bovine Serum (FBS, Biosera FB-1090/500) and 550 μL Leukaemia Inhibitory Factor (1000 units, Merck Millipore ESG1107).

Gelatin (Sigma-Aldrich G1890-100G), dissolved in distilled water to a 1% (w/v) solution, autoclaved and diluted to 0.1% in PBS (see below).

NDiff227 (N2B27, Takara Y40002).

Phosphate Buffered Saline (PBS with calcium and magnesium, Sigma-Aldrich D8662). SB431542 (100 mM SB43, use at 10 μM; Tocris Bioscience 1614).

Sterile reservoir (55 mL, STARLAB E2310-1010).

Trypsin-EDTA (0.05%, Gibco 25300-054).

U-bottomed non-tissue cultured treated 96-well plate (Grenier 650185).

XAV939 (10 mM TIN, use at 1 μM; Tocris Bioscience 3748).

Basic Method

Day 0: Culture Conditions Prior to Aggregation
Maintain mESCs in ESLIF medium (see Materials and Reagents) on gelatin-coated tissue culture-treated plastic (25 cm²flasks) in a humidified incubator at 37° C. and 5% CO₂.
Grow cells for at least 2 passages post-thawing before use.
Grow cells at 40-60% confluency.

Day 1: Generation of Aggregates (0-24 h)
Pre-warm PBS (+Ca²⁺, +Mg²⁺), ESLIF, N2B27 and Trypsin-EDTA in a 37° C. water bath.
Aspirate medium from the tissue culture flask and rinse gently with 5 mL PBS, twice.
Aspirate the PBS and add 1 mL of pre-warmed Trypsin-EDTA (0.05%) to dissociate the cells.
Incubate the flask at 37° C. for 60 seconds. Agitate the flask to detach the cells and strike the wall of the flask a few times with the palm of your hand if required.
Dissociate the colonies into a single cell suspension by drawing the cells into a P1000 micropipette tip and ejecting them against the base of the flask. This is crucial for accurate counting.
Neutralise the Trypsin-EDTA with 5 mL ESLIF and transfer to a 50 mL centrifuge tube.
Centrifuge for 5 minutes at ˜170×g.
Aspirate the supernatant and add 5 mL warm PBS.
Centrifuge for 5 minutes at ˜170×g.
Aspirate the supernatant and add 5 mL warm PBS.
Centrifuge for 5 minutes at ˜170×g.
Aspirate the PBS, minimising carry-over by tilting the tube and resuspend the pellet in 1 mL warm N2B27 using a 1 mL micropipette.
If required: Take a 100 μL aliquot of the cell suspension and dilute it in 900 μL N2B27 in a 1.5 mL Eppendorf tube (1:10) for counting.
Remove 10 μL from the (diluted) suspension and count the cells using a haemocytometer (or an automated cell counter, if available).
Determine the volume required for 3.75×10⁴ cells¹ for the SBR cell line and 5.00×10⁴ cells (For 400-cell aggregates, use 5.00×10⁴ cells/5 mL; for 300-cell aggregates, use 3.75×10⁴ cells/5 mL; for 200-cell aggregates, use 2.50×10⁴ cells/5 mL) for the Nodal-YFP line. Also calculate the volume of N2B27 required to give a final volume of 5 mL in each case.
Add the calculated volume of (diluted) cell suspension to the N2B27 and transfer the final 5 mL plating suspension to a sterile reservoir.
Pipette 40 μL of cell suspension into each well of a sterile, U-bottomed non-tissue culture-treated 96-well plate using a multichannel pipette.
Cover the plate with a lid and confirm the presence of single cells using an inverted bench-top microscope.
Incubate the cells in a humidified incubator at 37° C. and 5% CO₂ for 48 hours.

Day 2: Observation (24-48 h)
No action is required at this point, but the gastruloids should be inspected to confirm that the cells have formed single aggregates.
Small, satellite aggregates can indicate that too many cells have been plated in each well.

Day 3: Applying Stimuli (48-72 h)
Pre-warm N2B27 in a 37° C. waterbath.
Prepare secondary media in N2B27 to the following concentrations: Chiron, 3 μM; SB431542, 10 μM; XAV939, 1 μM.
Transfer 150 μL of secondary medium to each well of the 96-well plate using a multichannel pipette and a sterile plastic reservoir.
Incubate the gastruloids in a humidified incubator at 37° C. and 5% CO₂ for 24 hours.

Day 4: Changing Medium (72-96 h)
Pre-warm N2B27 in a 37° C. waterbath.
Remove 150 μL of culture medium from each well using a multichannel pipette, holding it at an angle and using a sterile reservoir for waste.
Add 150 μL of fresh, warm N2B27 to each well using a multichannel pipette.
Observe the fluorescence under the microscope (equipped for epifluorescence imaging).
Return the plate to the humidified incubator at 37° C. and 5% CO₂.

Day 5: Changing Medium (96-120 h)
Repeat the medium change as for Day 4, using pre-warmed N2B27.
Observe the fluorescence under the microscope (equipped for epifluorescence imaging).

Day 6: Observation (120 h)
Repeat the medium change as for Day 4, using pre-warmed N2B27.
Observe the fluorescence under the microscope (equipped for epifluorescence imaging).

Cardiac Organogenesis Method and Results

The “Cardiac Method” was based on the “Basic Method” provided above with the following modifications.

Gastruloids were transferred from 96-well plates to low-adherence 24-well plates at day 4. This brings a more than 4-fold higher volume of medium, resulting in an improved availability of nutrients and growth factors. Second, we modified the medium exchange procedure, introducing a half-medium change instead of a full change every day from day 5 onwards. This step is useful in maintaining a “conditioned” environment that promotes in vitro organogenesis. Third, we introduced shaking of cultures to extend gastruloid lifespan. When gastruloids are transferred to a shaking platform located in a classic cell culture incubator from day 4 from aggregation, they look healthier and can be grown for extended periods of time.

Gastruloids are by definition composed of cells from the three germ layers from which all organs of the body will develop. To elicit and/or enhance the first steps of organogenesis in culture, we applied organ-specifying factors to our cultures. As a proof of principle, we concentrated on the heart and blood, which are the first developing organs in the embryo.

We formulated a medium enriched in VEGF, Ascorbic Acid (AA) and basic FGF (bFGF), in which gastruloids are grown from day 4. We find that the combination of increased medium volume, shaking, modified medium exchange and addition of VEGF, AA and bFGF to our cell culture results in a very robust development of beating portions at the gastruloid anterior side 7 days after aggregation (FIG. 15B, C). These portions are positive for cardiac-specific Troponin T, a pan-cardiomyocyte marker (FIG. 15D). Notably, the development of the beating portion follows the same timing of expression of cardiac progenitor and differentiation genes of in vivo embryos, suggesting that cardiac developmental steps are faithfully reproduced (FIG. 15E).

Perhaps most importantly, we also observe the reproducible development of a network of Flk1/VEGFR2 positive cells in the anterior portion, resembling the formation of a vascular network (FIG. 16A,B). This aspect is particularly relevant since in vivo the heart develops in a concerted way with the vascular system. Cells start to express detectable levels of Flk1, an early marker of the cardiovascular system, at day 4 in culture, at the anterior side of the gastruloid, in a region which is always opposite to Brachyury expression. The vascular network shows positivity for CD31/PECAM1, a marker of endothelial cells (FIG. 16C). The network is formed around day 5 to day 7 from Flk1+ cells at the most anterior region of the gastruloid. This process is mediated by migratory movements accompanied by changes in cell shape, similarly to what happens in endothelial cell-mediated vasculogenesis. Furthermore, when plated on Matrigel™, isolated Flk1+ cells show a sprouting behavior akin to the vasculogenic behavior typically observed with endothelial cells.

Haematopoietic Organogenesis Method and Results

We have developed protocols to promote blood specification and capture both primitive and definitive haematopoiesis, with the potential to produce haematopoietic stem cells (HSC) in the absence of genetic manipulation.

The “Haematopoietic Method” was based on the “Basic Method” provided above with the following modifications, as illustrated in (FIG. 17A), with respect to:
(i) pre-treatment of ESC for 1 passage in na\ddot{v}e pluripotency conditions (PDO3, Chi and Leukaemia Inhibitory Factor, LIF) for reproducible production of elongating gastruloids;
(ii) usage of low adherence 96-well plates throughout the assay, so as to sustain gastruloid cultures for 7-10 days;
(iii) seeding of 250 ES cells and plate centrifugation to initiate aggregation;
(iv) pulsing with Activin A (100 ng/ml) as well as Chi 3 uM for 24 h after 2 days of aggregation;
(v) maintaining cells in FGF2 (5 ng/mL) and VEGF (5 ng/mL) with daily half-medium changes from day 3 onwards
optionally (vi) adding SCF (100 ng/ml) from day 5, Shh (20 ng/mL) from day 7 and/or Noggin (400 ng/ml) from days 8-9 of culture.

The resulting gastruloid cultures exhibit polarised expression of a Flk1-GFP reporter from 96 h, with subsequent nucleation of the Flk1-GFP expression and the formation of luminal-like structures, that are evident as a more complex network from 144 h onwards, and could correspond to vasculature (FIG. 17B).

Differentiating gastruloid cultures maintained in FGF2 and VEGF significantly up-regulate expression of haematopoietic, as well as endothelial markers (FIG. 17C), including production of adult haemoglobin (Hbb-b1) (FIG. 17D), suggesting the capacity to establish definitive haematopoiesis. From day 5 onwards of gastruloid culture in FGF2/VEGF conditions, it was possible to elicit the activity of haematopoietic progenitors (FIG. 17E), by testing dissociated gastruloids in classical colony-forming assays in methylcellulose-based semi-solid medium in the presence of a minimum cocktail of growth factors (SCF 100 ng/mL, IL3 10 ng/mL, hIL6 10 ng/ml and hEPO 3U/mL).

Flow cytometry analysis of dissociated Flk1-GFP-expressing gastruloids revealed the presence of CD41+ C-Kit+ cells and CD45+, cKIT+ and CD31+ cells, compatible with the development of early haematopoietic percursors from haemogenic endothelium. These cells were apparent from day 5 of gastruloid culture amongst Flk1-GFP+ cells, in particular at higher levels of GFP detection (FIG. 18A). Addition of Shh±SCF at day 5 of culture promoted both Flk1-GFP expression and the prevalence of CD41+ C-Kit+ cells (FIG. 18B). Consistent with the specification of an early haematopoietic progenitor, colony-forming assays generated from Shh±SCF-supplemented gastruloid cultures consistently contain a small percentage of mixed-lineage colonies that give rise to mixed erythroid/myeloid colony reflecting the presence of multlineage progenitors in day 6 gastruloid cultures upon addition of Shh. (FIG. 18C).

### Example 3

### Further Data Regarding Cardiac Organogenesis

During embryogenesis, the formation of the heart is mediated by waves of different progenitors, which are typically defined as first and second heart field (FHF and SHF, respectively). FHF and SHF progenitors differ in their kinetic of proliferation and differentiation, in the expression of typical markers and, most importantly, in their prospective contribution to the different heart portions (Miquerol and Kelly , w2013). Since progenitors from the two different heart fields will give rise to different portions of the heart and outflow tract, it is an important point that features of both FHF and SHF are recapitulated in Gastruloids. As shown in FIG. 19, analysis of FHF (Tbx5) and SHF (IsI1-Tbx1) markers in Gastruloids show a markedly higher expression compared to undifferentiated mESCs (FIG. 19A-C). Moreover, histological section analysis revealed expression of these markers in the anterior portion of the Gastruloid, in a mutually exclusive fashion (FIG. 19D-F). This feature suggests that both FHF and SHF progenitors can be recapitulated in Gastuloids.

A second remarkable feature of Gastruloids is that the formation of a beating portion can pass through spatially organized domains which are similar to those observed in embryos. Specifically, in mouse embryos at E7.5, cardiac progenitors are located in a crescent-like domain, namely the cardiac crescent (Miquerol and Kelly). From this crescent, progenitors will fuse at the midline to give rise to the heart tube, the first beating structure which develops during cardiogenesis (around E8-E8.5 in the mouse). As shown in FIG. 20, Gastruloids can develop a beating portion passing through a crescent-like domain (FIG. 20A, B, E, F). These cTnT positive crescent-like structures will then condense (FIG. 20C) into a smaller domain of expression (FIG. 20D), corresponding to the beating portion. This feature is reminiscent to the formation of a cardiac crescent in mouse embryos (Ivanovitch et al., 2017).

### Example 4

### Further Data Regarding Hematopoiesis

Detection of Gastruloid Derived Aortic Clusters

FIG. 21 shows gastruloid derived aortic clusters of pre HSCs (hematopoietic HSCs). The Figure shows an example of an aortic cluster from a mouse embryo from the indicated reference and, on the right, images from cluster like structures from 192 hrs gastruloids in which there is an attached cluster of cells co-expressing CD31 (Pecam), CD45 and c-Kit. The expression of these markers together and in the illustrated cellular configuration provides strong evidence supporting the generation of pre-HSCs.

Engraftment Experiment

FLK1-GFP cells were originally generated in a C57131/6 background, and express the CD45.2 isoform of the pan-haematopoietic marker CD45.

Dissociated cells from 192 h gastruloids cultured under FGF2/VEGF, with or without a 24 h pulse of SHH between 144 and 168 h, were washed and resuspended in PBS/BSA and injected intra-tail vein into irradiated CD45.1 recipient mice. Animals were irradiated with a total of 8Gy 1-2 h prior to injection and gastruloid-derived cells were injected in a mixture with accessory bone marrow CD45.1 cells to support the early haematopoietic recovery post-irradiation. In some cases, 192 h-gastruloids from both culture conditions were pre-sorted by flow cytometry on CD45 expression with the aim of injecting a pure population of cells in the haematopoietic lineage. Animals injected with whole gastruloid culture received the same equivalent amount of CD45+ cells.

Engraftment was monitored in the peripheral blood at 8 weeks by staining of a cell suspension pre-treated with red blood cell lysis buffer, with antibodies against CD45.1 and CD45.2 so as to distinguish host (CD45.1) from graft (CD45.2) cells. Some animals were sacrificed at 10 weeks and bone marrow and spleen collected and stained as described for peripheral blood.

All experiments were performed against a control CD45.1 animal that did not receive gastruloid cells.

Results

Week 9 Mouse GT7 (unsorted VEGF), GT9 (unsorted Shh), GT11 (sorted VEGF) and GT14 (no injection) have been terminated and FACS analysis was performed on CD45.1-PE and CD45.2-APC-Cy7.

Bone marrow cells were harvested for FACS. The results of which are shown in FIG. 22.

### Example 5

### Epi-gastruloids

Epiblast Stem Cells (EpiSCs) are epithelial and have several properties that make them different from naïve ESCs. These cells are in a state of what is known as ‘primed pluripotency’ and part of the interest in these cells is derived from their similarity to hESCs. EpiSCs are unable to form Gastruloids under standard conditions. However, the invenotors have developed a protocol that allows EpiSCs to form a different kind of Gastruloid structure, that we call EpiGastruloid. This protocol required an adaptation to the conditions of the EpiSCs and involves a pretreatment that lasts three days in adherent culture and aggregation in a combination of the Wnt agonist CHI and FGF2. Under these conditions, EpiSCs form a polarized structure that grows over time. We find that ESCs do not produce EpiGastruloids. This differential behaviour allows the two types of Gastruloids to discern between different states of pluripotency and we have shown this.

EpiGastruloids can be used, in parallel with standard Gastruloids, to test the state of different stem cell populations and as a source of more posterior fates.

Method

Pretreatment: Start with EpiSC (ES cells after 3 passages in bFgf/Activin) (bFGF: 12 ng/mL and Activin 25 ng/mL).

Day 0: Plate 5.10⁵ EpiSC in a well of 6WP coated with Fibronectin in bFgf/Activin medium. (bFGF: 12 ng/mL and Activin 25 ng/mL)

Day 1: Change medium with N2B27+bFGF (bFGF:20 ng/mL concentration).

Day 2: Change medium with N2B27+bFGF+CHI (bFGF:20 ng/mL and CHI:3 uM).

Aggregation: Low adherent 96w plates (Greiner) were used.

Confluent cells were washed with PBS and Accutase was used to detach the cells. The cells were spun, counted and 350 cells were plated per 96w plate in 40 ul of Fgf and CHI medium (bFGF: 20 ng/mL and CHI: 3 uM). 150 ul FGF+CHI medium was added after 48 hours of plating cells and after that, 150 ul of medium was changed every day. The cells were imaged every 24 hours, from 24 hours-120 hours.

### Example 6

### Effects of Embedding Gastruloids in Matrigel

Materials and Methods—See Example 1, Materials and Methods, varied as described below.

Results

FIG. 23. Embedding gastruloids in Matrigel triggers different outcomes depending on the age of embedding. Some examples are shown of the outcome of embedding in 75% matrigel, which is representative. Embedding at 72 hrs after aggregation aborts or leads to defects in patterning. Embedding at 96 hrs after aggregation tends to split the axially elongating structure and produce twinning. Embedding at 120 hrs after aggregation leads to long aggregates and the frequent (>50%) appearance of epithelial cysts periodically arranged from posterior to anterior.

FIG. 24. Examples of 144 hrs old gastruloids embedded in Matrigel as indicated, at 120 hours after aggregation exhibiting periodically arranged epithelial cysts (top) compared to non-embedded (suspension cultured) gastruloids. Inset top right shows a magnification and detail of these structures which have been shown to express somite specific genes (van den Brink et al. 2019)

### Example 7

### Impact of Preculture Conditions on the Frequency of Elongated Gastruloids

Materials and Methods—See Example 1, Materials and Methods, varied as described below.

Results

FIG. 25. Impact of preculture conditions on the frequency of elongated gastruloids. Left, quantification of different kinds of treatment before aggregation with two different starting numbers of cells. Notice that in all cases, pretreatment with 2i (Chiron and PD03) just before aggregation reduces significantly the number of improperly patterned gastruloids with the best results if cells grown in ES LIF (ES) are then transferred before forming the aggregate to 2i; addition of LIF to the 2i, provides additional help. On the right, some examples of a typical experiment at 120 hrs after aggregation.

### Example 8

### PGC-Like Cell Formation in Gastruloids

Materials and Methods—See Example 1, Materials and Methods, varied as described below.

Results

FIG. 26—PGC-like cell formation in gastruloids. (a) Co-expression of Blimpl-GFP and AP2gamma in 120 h gastruloids made from the Blimpl-GFP cell line. (b) Flow cytometric analysis of Blimp1-GFP gastruloids at 120 h, showing co-expression of beta-Integrin, SSEA1 and Blimp1-GFP. (c) Gastruloids made from the Stella-GFP,Esg1-tomato (SGET) cell line at 120 h. (d) Flow cytometric analysis of co-expression of Stella-GFP and Esg1-Tomato in 120 h SGET gastruloids. (e) Co-expression of Stella and AP2gamma in 120 h SGET gastruloids.

### Example 9

### Embryonic Organoids Recapitulate Early Heart Organogenesis

Summary

Organoids are powerful in vitro models for the study of tissue development, physiology and disease. However, existing organoids are derived using methodologies that disrupt inductive 3D tissue-tissue interactions that play an indispensable role during native organogenesis (Tom et al., 1997; and Harvey et al., 2002). It has thus not yet been possible to recreate organogenesis approximating the spatial and temporal fidelity found in the embryo (Rossi et al., 2018). Here we show that when stimulated with key cardiogenic factors, embryonic organoids (van den Brink et al., 2014; and Beccari et al., 2018) can be coaxed to robustly undergo fundamental steps of early heart organogenesis (Miquerol and Kelly 2013) in a spatiotemporally accurate manner. In particular, these organoids support the formation of Mesp1⁺ cardiac progenitor cells that persist anteriorly, the generation of Flk1⁺ bipotent cardiovascular progenitors, and the formation of first and second heart field compartments. Cardiac progenitors self-organize into an anterior domain reminiscent of a crescent, which further condenses to form a beating cardiac tissue. Similar to what happens in vivo, in embryonic organoids the heart primordium forms anteriorly, in close proximity to an epithelial tissue akin to the primitive gut tube, from which it is separated by an endocardial-like layer. These findings highlight the surprising potential of aggregated, free-floating embryonic stem cells to progress beyond the previously characterized early developmental stages preceding organogenesis. This platform provides new perspectives to study heart development in vitro with unprecedented detail and throughput.

Methods

Cell Culture

mESCs were cultured at 37° C., 5%CO₂ in DMEM supplemented with 10% Embryonic Stem Cell qualified FBS (Gibco), NEAA, Sodium Pyruvate, β-mercaptoethanol, 3 μM CHI99201 (Chi), 1 μM PD025901 and 0.1 μg ml⁻¹ LIF. Gata6-Venus (Freyer et al., 2015), Flk1-GFP (Brutsaert 2003), and Mesp1-GFP (Bondue et al., 2011) cells were cultured on gelatinised tissue-culture flasks; Soxl-GFP::Brachyury-mCherry (Deluz et al., 2016) cells on tissue-culture flasks without coating. If not differently specified, Sox1-GFP::Brachyury-mCherry (Deluz et al., 2016) cells were used for our experiments. HUVECs were cultured in EGM-2 medium (Lonza). All cells were routinely tested for Mycoplasma with Mycoalert mycoplasma detection kit (Lonza) or by PCR.

Gastruloid Culture

Gastruloids were generated as previously described (Baillie-Johnson et al., 2015). Briefly, 300-700 mESCs were plated in 40 μl N2B27 in 96-well Clear Round Bottom Ultra-Low Attachment Microplates (7007, Corning). After 48 h, 150 μl of N2B27 containing 3 μM Chi were added to each well. After 72 h, medium was changed with N2B27. Starting from 96 h, the protocol was optimized as described in FIG. 31a. At 96 h, gastruloids were transferred in Ultra-Low Attachment 24-Well Plates (3473, Corning) in 100 μl of medium, plus 700 μl of fresh N2B27 containing 30 ng ml⁻¹ bFGF (PMG0034, Gibco), 5 ng ml⁻¹ VEGF 165 (PHC9394, Gibco) and 0.5 mM L-ascorbic acid phosphate (013-12061, Wako) (N2B27+++) and cultured on an orbital shaker placed at 37° C., 5%CO₂ at 100 rpm (VWR mini shaker). From 120 h on, half medium was changed daily. Unless differently specified, N2B27+++was applied from 96 to 144 h, while from 144 h to 168 h N2B27 was used for medium change.

Live Imaging and Cell Tracking

Bright-field live imaging of beating gastruloids was performed with a Nikon Ti inverted microscope equipped with an incubation chamber at 37° C., 5% CO2. Light sheet live imaging of Flk1-GFP and Mesp1 gastruloids was performed with a prototype of LS1 live inverted light sheet microscope (Viventis Microscopy Sari, Switzerland), at 37° C., 5%CO₂. A volume of 150-200 μm was acquired with a Z spacing of 2-3 μm between slices and pictures were captured every 20 min for Flk1 gastruloids and every 10min for tracking of Mesp1 cells. Flk1 light sheet video montages were obtained with the Arivis Vision4D software. To track Mesp1⁺ cells in Gastruloids from 96 to 120 h, LS1 live light sheet images were processed with the Fiji Mastodon plugin, using a semi-automatic tracking. Subsequently, Mastodon files were exported for Mamut, and the Fiji Mamut plugin was used to display cell tracks as shown in FIG. 27.

Immunofluorescence, Confocal and Light Sheet Imaging on Fixed Samples

Immunofluorescence on whole mount gastruloids was performed as previously described (Baillie-Johnson et al., 2015). Briefly, gastruloids were washed in PBS and fixed in 4% PFA for 2 hours at 4° C. while shaking. Samples were washed 3 times in PBS and 3 times (10 minutes each) in blocking buffer (PBS, 10%FBS, 0.2%Triton X-100), then blocked for 1 h at 4° C. in blocking buffer. Gastruloids were then incubated O/N with primary antibodies in blocking buffer, at 4° C. while shaking. The day after, gastruloids were washed 4 times (20 minutes each) with blocking buffer, at 4° C. while shaking, and incubated O/N with secondary antibodies and DAPI (2 μg ml⁻¹, Sigma-Aldrich) in blocking buffer, at 4° C. while shaking. The day after, gastruloids were washed for 1 h with blocking buffer, at 4° C. while shaking, then rinsed in PBS, 0.2%FBS, 0.2% Triton X-100 and mounted on Superfrost plus glass slides (ThermoFisher) with Floromount-G for confocal imaging (Southern Biotech). The following primary antibodies were used: mouse anti-Gata4 (1:500, Santa Cruz Biotechnology, G-4); chicken anti-GFP (1:750, Ayes Labs); goat anti-Brachyury (1:300, Santa Cruz Biotechnology, C-19); rat anti-CD31 (1:100, BD, MEC 13.3), mouse anti-cardiac troponin T (1:100, ThermoFisher, 13-11), rabbit anti-E-Cadherin (1:500, Cell Signaling, 24E10). The following secondary antibodies were used: donkey anti-chicken 488 AlexaFluor (1:500, Jackson ImmunoResearch); donkey anti-goat AlexaFluor 568 (1:500, ThermoFisher); goat anti-rat AlexaFluor 568 (1:500, ThermoFisher); goat anti-mouse AlexaFluor 647 (1:500, ThermoFisher); donkey anti-rabbit 568 (1:500, ThermoFisher). Confocal pictures were acquired with a Zeiss LSM 700 inverted confocal microscope equipped with a Axiocam MRm black and white camera in the EPFL bioimaging and optics facility. For light sheet imaging (FIG. 30), samples were mounted in 1% low-melt agarose and cleared overnight with CUBIC mount solution (Lee et al., 2016). Light sheet imaging was performed on a Zeiss Light-sheet Z1 microscope equipped with a Plan-Neofluar 20×/1.0 Corr nd=1.45 objective. Light sheet images were further processed with Imaris software, for 3D rendering and surface generation.

RNA Extraction and qRT-PCR

RNA was extracted from gastruloids with the RNeasy Micro kit (Qiagen), according to manufacturer's instructions and quantified with a spectrophotometer (ND-1000, Nanodrop). 1 μg of RNA was reverse-transcribed with the iScript cDNA Supermix kit (Biorad). cDNA was diluted 1:10 and 1.5p1 of cDNA per reaction were used, in a total volume of 10 μl. 384 plates were prepared using a robotized liquid handling platform (Hamilton Microlab Star). qPCR was run with a 7900HT Fast PCR machine (Applied Biosystems), using Power SYBR Green PCR Master Mix (Applied Biosystems), with an annealing temperature of 60° C. Gene expression was normalized on β-actin expression. Relative fold expression was calculated with the 2-AACT method. 500 nM of the following primers were used: Mesp1 FOR GTCTGCAGCGGGGTGTCGTG; Mesp1 REV CGGCGGCGTCCAGGTTTCTA; Nkx2.5 FOR CACATTTTACCCGGGAGCCT; Nkx2.5 REV ACCAGATCTTGACCTGCGTG; HCN4 FOR GTGGGGGCCACCTGCTAT; HCN4 REV GTCGGGTGTCAGGCGGGA; a-actinin FOR GGGCTATGAGGAGTGGCTATT; α-actinin REV AGTCCTTCTGCAGCAAGATCT; RyR2 FOR TGCATGAGAGCATCAAACGC; RyR2 REV CGCGGAGAGAGGCATTACAT; Tbx5 FOR GGCATGGAAGGAATCAAGGTG; Tbx5 REV TTTGGGATTAAGGCCAGTCAC; Tbx1 FOR CTGTGGGACGAGTTCAATCAG; Tbx1 REV TTGTCATCTACGGGCACAAAG; IsI1 FOR ATGATGGTGGTTTACAGGCTAAC; IsI1 REV TCGATGCTACTTCACTGCCAG; FGF10 FOR TCAGCGGGACCAAGAATGAAG; FGF10 REV CGGCAACAACTCCGATTTCC; β-actin FOR CTGTCGAGTCGCGTCCACC; β-actin REV CGCAGCGATATCGTCATCCA.

RNAscope

For RNAscope, gastruloids were washed in PBS and fixed 0/N in 4% PFA, at 4° C. while shaking. The day after, samples were washed 3 times in PBS and included in HistoGel (ThermoFisher) blocks. HistoGel blocks were then processed with a Tissue-Tek VIP 6 Al Vacuum Infiltration Processor (Sakura) and included in paraffin. Paraffin blocks were cut at 4□m with a Hyrax M25 microtome (Zeiss). RNA-scope was performed with the ACDBio Manual assay kit using RNAscope Probe-Mm-Tbx1 (481911), RNAscope Probe-Mm-IsI1-C3 (451931-C3) and RNAscope Probe-Mm-Tbx5-C2 (519581-C2) probes, according to manufacurer's instructions. Polr2a-C1, Ppib-C2 and Ubiquitin-C3 probes were used as positive and negative controls. Pictures were acquired with an upright Leica DM5500 microscope equipped with a CCD DFC 3000 black and white camera.

FACS Analysis and Cell Sorting5

In order to perform FACs analysis and cell sorting, gastruloids were collected, washed in PBS, and digested in 4 mg ml⁻¹ dispase I (Roche), 3 mg ml⁻¹ collagenase IV (Gibco) and 100 μg ml⁻¹ DNase I (Roche) in PBS (2 digestion cycles at 37° C., 5 min each; gentle pipetting was applied between the two cycles to mechanically dissociate the gastruloids). Digestion was blocked with DMEM containing 10% FBS, then samples were centrifuged and the cell pellet was resuspended in sorting buffer (PBS, 5%FBS, 1 mM EDTA, 1%P/S) for antibody staining. Samples were incubated for 1 h on ice with antibodies, and 30 min on ice with Aqua live/dead fixable dead cell stain kit (405/525 nm, Invitrogen) or 10 min on ice with DAPI. Unstained, FMO and single color samples were used as controls. The following antibodies were used: anti CD31-PE 1:1200 (BD, MEC 13.3), anti VEGFR2/Flk1-APC 1:200 (Biolegend, Avas12); anti CXCR4-APC 1:100 (BD, 2611). Samples were analysed with a BD LSR II flow cytometer. Cell sorting was performed using a BD FACSAria Fusion cell sorter.

Angiogenesis Assay

For the angiogenesis assay, Flk1-GFP gastruloids at 168 h were collected and digested as described for FACs analysis. Flk1⁺ and Flk1⁻ cells were isolated through cell sorting with a BD FACSAria Fusion cell sorter. 5 10⁴ cells per condition were plated in IBIDI μ-angiogenesis slides pre-coated with 10 μl reduced growth factor Matrigel (Corning) in the lower chamber. Undifferentiated mESCs and HUVEC were used as negative and positive controls, respectively. Live imaging of tube formation was performed with a Nikon Ti inverted microscope equipped with an incubation chamber at 37° C., 5% CO₂, with acquisitions every 15 minutes.

Calcium Imaging

To image calcium fluxes, gastruloids were incubated for 1 h with 8 μM Cal-520 (AAT Bioquest) at 37° C., 5% CO₂. Gastruloids were then transferred to fresh medium before imaging. Imaging was performed with a Light sheet Z1 microscope (Zeiss) equipped with an environmental chamber to maintain gastruloids at 37° C. and 5% CO₂. For imaging, gastruloids were embedded in 1% low melt agarose and the chamber was filled with culture medium. Nifidepine (Sigma Aldrich, 10 μM) and Isoproterenol (Isoprenaline hydrochloride, Sigma 15627, 1 μM) were added with a syringe directly to the imaging chamber during acquisition. The analysis of calcium spikes was performed with the Fiji Stacks-plot Z-Axis profile plugin. The baseline intensity was normalized to the minimum value over 10 sec. The ratio of fluorescence intensity to baseline intensity was calculated and results are shown as the percentage of increase over the baseline, which shows the relative changes in intracellular Ca²⁺.

Crescent Analysis

Analysis of crescent geometrical properties was performed using a custom-made Matlab script for Imaris files with a Imaris XT feature and EasyXT. Initially, we generated artificial shapes to be used as reference for defined geometrical metrics. Using a custom script (crescent generator.m), 3D objects were generated to mimic crescent structures with different characteristics. To do so, the script creates two spot objects, makes channels from these spots and finally subtracts one channel to the other. To analyse surfaces, 3D images from non-beating organoids at 144 h and beating organoids at 168 h were acquired with a Light sheet Z1 microscope (Zeiss) after clearing, as described above. For each individual stack, a surface was created using a dedicated user interface (GUI DetectAndAnalyze.m), defining the object that should be created for each channel (settings .m). Due to variability in background and signal intensity, the threshold (absolute intensity) was adjusted manually for each surface. Using a custom script (makeCroissantMeasure_final.m) geometrical measurements were computed and the results exported in csv table. In the graph, we plot the measure of Spareness, which is described as the ratio between the volume of the object and the volume of the best fitted ellipsoid. All scripts and settings used for analysis are available at Zenodo.org.

Statistics

All data shown in column graphs are expressed as mean±SD, apart from the graph showing Calcium spikes frequency, which is expressed as mean±whiskers from min to max. All other graphs show single data points. Statistical analysis between two columns was performed using two-tailed unpaired Student's t test, whereas data containing more than two experimental groups were analyzed with one-way analysis of variance followed by Bonferroni's test. To calculate the significance of the percentage of increase over baseline frequency after Isoproterenol administration, we applied a one sample t test. Statistical significance was calculated using the Graphpad Prism software, that was also used to generate all graphs. *P<0.05; **P<0.01; ***P<0.001; confidence intervals 95%; alpha level 0.05.

Results

Stem cell-derived organoids self-organize into complex structures mimicking aspects of the architecture, cellular composition and functionality of tissues found in real organs (Rossi et al., 2018; Sasai 2013; Clevers 2016; and Lancaster and Knoblich 2014). While most organoids are focused on the recapitulation of specific features of adult organs, embryonic organoids can capture key processes happening during early embryonic development, from the pre-implantation blastocyst (Rivron et al., 2018) to early post-implantation development (Harrison et al., 2017; Shao¹ et al., 2017; Shao² et al., 2017; and Sozen et al., 2018) and gastrulation (as shown in this disclosure).

When cultured for 144 h or longer in N2B27 medium, gastruloids occasionally form a beating domain that is exclusively located within their anterior region (38.5±29.3% at 168 h) (FIG. 27a). The location and activity of the structure suggested that it might correspond to a cardiac primordium. We tested whether well-known cardiogenic factors (Rajala et al., 2011) could increase the frequency of this event by adding basic fibroblast growth factor (bFGF), ascorbic acid and vascular endothelial growth factor 165 (VEGF) (Kattman et al., 2006), singly or in combination, and increasing nutrient and growth factor availability through volume optimization and shaking (FIG. 27a, FIG. 31a). In these culture conditions (termed N2B27+++), the frequency of beating gastruloids increased by more than a factor of two (87.2±15.6% at 168 h) (FIG. 27a,b). We noticed that exposure to cardiogenic factors was most effective when applied in combination and between 96 and 144 h (FIG. 31a-f). We thus kept this protocol for the following experiments. Importantly, culture in N2B27+++ did not alter the polarization of the gastruloids, the extent of their elongation (FIG. 31i-j), nor the timing of the emergence of the beating domains (FIG. 27a, FIG. 31b-d) compared to standard conditions. Staining for Gata4 and cardiac troponin T (cTnT) confirmed that the beating structure was related to a cardiac structure (FIG. 27c).

In cardiomyocytes, physical contraction is coupled to electrical excitation through intracellular changes of Ca²⁺ (Tyser et al., 2016). To evaluate the functionality of the gastruloid cardiac domain, we thus assessed calcium transients by live gastruloid imaging via light sheet microscopy (at 168 h). Image analysis revealed rhythmic calcium spiking in beating areas, with a frequency that is comparable to beating rates observed in embryos from the crescent stage to the linear heart tube (Tyser et al., 2016) (FIG. 27d-f). Exposure to drugs interfering with calcium transport, including the I-type calcium channel blocker nifidepine and the β-adrenergic agonist isoproterenol, resulted in a complete inhibition of transients (FIG. 32a) or an increase in spiking frequency (FIG. 32b,c), respectively. These data demonstrate that the cardiac compartments found in late gastruloids (168 h) exhibit Ca²⁺-handling properties compatible with functional cardiomyocytes.

To understand if the cardiac portion forms through the recapitulation of developmentally relevant processes, we first analyzed the temporal expression of key genes involved in cardiovascular specification (FIG. 27g-k). Similarly to what happens during embryonic development (Saga et al., 1996; and Lescroart et al., 2014), the first cardiac gene upregulated was Mesp1, which was expressed around 96 h and then rapidly downregulated (FIG. 27g, FIG. 32d,e). Using a Mesp1-GFP reporter ESC line (Bondue et al., 2011), we observed that Mesp1 expression started in a mosaic-like manner at 96 h, with Mesp1 positive cells first being confined at the anterior side before slowly disappearing after 120 h (FIG. 27m). At the same time, and coincidently with elongation and formation of an anterior-posterior axis, Gata6-expressing cells in gastruloids generated from Gata6-Venus ESCs21 localized anteriorally, opposite to the Brachyury positive pole (FIG. 27n). Gata6 expression was maintained over time (FIG. 32f,g). From this stage onwards, the early differentiation genes Nkx2-5 and HCN4 were increasingly expressed (FIG. 27h,i), followed by genes marking mature cardiomyocytes (α-actinin, Ryr2) (FIG. 27j,k). This sequence of gene expression shows that gastruloids, stimulated with cardiogenic factors, recapitulate the temporal and spatial gene expression dynamics of cardiac development, from the specification of cardiac progenitors to the formation of a beating cardiac structure.

During embryonic development, endothelial cells are a prerequisite for cardiomyocyte maturation, function and survival, through their continuous cross-talk in the developing heart (Brutsaert et al., 2003). For this reason we tested whether such tissue-tissue interaction could potentially take place in developing gastruloids, focusing on cardiovascular progenitors expressing the well-known marker Flk1 (also known as Kdr or Vegfr2) (Kattman et al., 2006). In 96 h gastruloids derived from a Flk1-GFP reporter ESC line (Jakobsson et al., 2010), Flk1 was expressed at the anterior pole opposite to Brachyury (FIG. 28a). Over time, Flk1 expression persisted in the anterior portion of the gastruloids (FIG. 28b, FIG. 33a,b), and Flk1 positive cells started to form a vascular-like network of spindle-shaped cells (FIG. 28c) staining positive for the endothelial marker CD31 (FIG. 28d, FIG. 33c). In an in vitro angiogenesis assay, Flk1 positive cells, isolated by fluorescence activated cell sorting (FACS) from 168 h gastruloids and plated on Matrigel, formed vascular-like networks similar to human umbilical vein endothelial cells (HUVECs) (FIG. 28e, FIG. 33d). Collectively, these results suggest that gastruloids comprise regions that develop into a vascular-like compartment, concomitant to undergoing cardiac development.

A key feature of cardiogenesis is a requirement for a coordinated interaction between two distinct mesodermal progenitor populations: the first heart field (FHF), that contributes to the left ventricle and part of the atria, and the second heart field (SHF) that gives rise to the outflow tract, right ventricle and part of the atria (Harvey et al., 2002; and Miquerol and Kelly 2013). After migration from the posterior primitive streak, FHF progenitors form the cardiac crescent and early heart tube anteriorly. SHF progenitors, originating from cardiopharyngeal mesoderm (Cortes et al., 2018), are characterized by delayed differentiation and are located medially to the crescent to then be involved in heart tube elongation. Each of these populations is characterized by a specific pattern of gene expression that we see recapitulated in our embryonic organoids, where we observed expression of markers of the FHF (Tbx5) and SHF (Tbx1) (FIG. 29a-c) in mutually exclusive cell populations, and IsI1 mostly overlapping with Tbx1 expression (FIG. 29d). To corroborate the presence of progenitor populations from both heart fields in our organoids, we used a protocol by Andersen et al. (2018) for FACS-isolating SHF progenitors based on the expression of the C—X—C chemokine receptor type 4 (CXCR4) (FIG. 29e). We observed that Gata6+/CXCR4+ cells express higher levels of SHF markers (Tbx1, IsI1 and FGF10), but low or unchanged levels of FHF markers (Nkx2-5, Tbx5 and HCN4) compared to Gata6+/CXCR4−, confirming their SHF identity (FIG. 29f-k). Together, these data show that late gastruloids contain key cell types that in vivo are involved in first and second heart field-based cardiogenesis. Importantly, unlike what was previously shown for precardiac spheroids (Andersen et al., 2018)), these cell populations emerge in an embryo-like spatially organized organoid.

In the mouse embryo, after their specification, cardiac progenitors migrate antero-laterally and progressively fuse at the midline to define the first morphologically identifiable heart structure, the cardiac crescent around E7.5 (Miquerol and Kelly 2013). Then, morphogenetic movements associated with foregut closure result in formation of a linear heart tube by E8.5 (Miquerol and Kelly 2013). We explored whether gastruloids stimulated with cardiogenic factors can capture these morphological hallmarks of cardiogenesis. Remarkably, in gastruloids cultured for 144 h to 168 h, we observed a recapitulation of these events. Around 144 h, cTnT positive cardiomyocytes were organized in crescent-like domains (FIG. 30a). Further development led to the formation of denser crescent-like structures with beating areas, leading to beating epithelial protrusions on the anterior portion of gastruloids at around 168 h. Similarly to mouse embryos (Tyser et al., 2016; Ivanovitch et al., 2017; and Le Garrec et al., 2017), these phenotypes can be observed in gastruloids within a time window of 24 h, and can be aligned to progressive morphological stages of cardiac development of the embryo between E7.5 and E8.5 (FIG. 4b). Indeed, a comparative volume analysis of the cTnT positive gastruloid domains with defined artificial shapes reveals a gradual transition from an almost spherical to a crescent-like (144 to 168 h) and then concave shape (168 h) (FIG. 30c). These results highlight a remarkable capacity of embryonic organoids to promote spatially and temporally orchestrated morphogenetic processes involved in the early stages of heart development.

The presence of a crescent-like structure and its evolution to a coherent beating group of cells, led us to test whether this was accompanied with an association between the crescent and anterior endoderm, as is the case in the embryo (Ivanovitch et al., 2017; and Lough and Sugi 2000). In gastruloids, primitive gut tube-like expression patterns (marked by Sox17, Shh, Sox2, CDX2 and E-cadherin) have already been described (Beccari et al., 2018). Strikingly, the cTnT positive cardiac domain in 168 h gastruloids was exclusively located in proximity to the anterior side of the gut tube (n=14/14), with a CD31 positive endocardial-like layer in between (FIG. 30d,e). This is highly reminiscent of the spatial arrangement of anterior structures in the developing embryo ((Ivanovitch et al., 2017), and it will be interesting to study in the future whether interactions between these tissues play a functional role in the development of the cardiac structures in this system.

These results show that embryonic organoids can be stimulated to recapitulate key steps of early cardiac development in vitro that in vivo require polarized interactions between different primordia. These interactions are critical for achieving architectural and compositional complexity that are absent in conventional organoids, but appear to be provided by the multi-axial properties and spatial organization of different embryonic tissues characteristic of gastruloids. In our case, we have steered these interactions to generate a cardiac primordium that serves as a basis for the development of an embryonic heart.

### REFERENCES

Andersen, P. et al. Precardiac organoids form two heart fields via Bmp/Wnt signaling. Nat. Commun. 9, 3140 (2018).

Baillie-Johnson, P., et al., Generation of aggregates of mouse ES cells that show symmetry breaking, polarisation and emergent collective behaviour. JOVE, 2015. doi: 10.3791/53252(105).

Beccari, L. et al. Multi-axial self-organization properties of mouse embryonic stem cells into gastruloids. Nature 562, 272-276 (2018).

Berge, ten, D. et al. Wnt Signaling Mediates Self-Organization and Axis Formation in Embryoid Bodies. Cell Stem Cell 3, 508-518 (2008).

Bondue, A. et al. Defining the earliest step of cardiovascular progenitor specification during embryonic stem cell differentiation. J. Cell Biol. 192, 751-765 (2011).

Brutsaert, D. L. Cardiac endothelial-myocardial signaling: its role in cardiac growth, contractile performance, and rhythmicity. Physiol. Rev. 83, 59-115 (2003).

Clevers, H. Modeling Development and Disease with Organoids. Cell 165, 1586-1597 (2016).

Cortes Claudio, Francou Alexandre, De Bono Christopher & Kelly Robert G. Epithelial Properties of the Second Heart Field. Circ. Res. 122, 142-154 (2018).

Deluz, C. et al. A role for mitotic bookmarking of SOX2 in pluripotency and differentiation. Genes Dev. 30, 2538-2550 (2016).

Freyer, L. et al. A loss-of-function and H2B-Venus transcriptional reporter allele for Gata6 in mice. BMC Dev. Biol. 15, 38 (2015).

Harrison, S. E., Sozen, B., Christodoulou, N., Kyprianou, C. & Zernicka-Goetz, M. Assembly of embryonic and extraembryonic stem cells to mimic embryogenesis in vitro. Science 356, (2017).

Harvey, R. P. Patterning the vertebrate heart. Nat. Rev. Genet. 3, 544-556 (2002).

Ivanovitch, K., Temiño, S., and Torres, M. (2017). Live imaging of heart tube development in mouse reveals alternating phases of cardiac differentiation and morphogenesis.

Jakobsson, L. et al. Endothelial cells dynamically compete for the tip cell position during angiogenic sprouting. Nat. Cell Biol. 12, 943-953 (2010).

Kattman, S. J., Huber, T. L. & Keller, G. M. Multipotent flk-1+ cardiovascular progenitor cells give rise to the cardiomyocyte, endothelial, and vascular smooth muscle lineages. Dev. Cell 11, 723-732 (2006).

Lancaster, M. A. & Knoblich, J. A. Organogenesis in a dish: Modeling development and disease using organoid technologies. Science 345, 1247125 (2014).

Le Garrec, J.-F. et al. A predictive model of asymmetric morphogenesis from 3D reconstructions of mouse heart looping dynamics. eLife 6,

Lee, E. et al. ACT-PRESTO: Rapid and consistent tissue clearing and labeling method for 3-dimensional (3D) imaging. Sci. Rep. 6, 18631 (2016).

Lescroart, F. et al. Early lineage restriction in temporally distinct populations of Mesp1 progenitors during mammalian heart development. Nat. Cell Biol. 16, 829-840 (2014).

Lough, J. & Sugi, Y. Endoderm and heart development. Dev. Dyn. 217, 327-342 (2000). Miquerol, L., and Kelly, R. G. (2013) Organogenesis of the vertebrate heart. Wiley Interdiscip. Rev. Dev. Biol. 2, 17-29.

Rajala, K., Pekkanen-Mattila, M. & Aalto-Setala, K. Cardiac Differentiation of Pluripotent Stem Cells. Stem Cells Int. 2011, (2011).

Rivron, N. C. et al. Blastocyst-like structures generated solely from stem cells. Nature 557, 106-111 (2018).

Rossi, G., Manfrin, A. & Lutolf, M. P. Progress and potential in organoid research. Nat. Rev. Genet. 19, 671 (2018).

Saga, Y. et al. MesP1: a novel basic helix-loop-helix protein expressed in the nascent mesodermal cells during mouse gastrulation. Dev. Camb. Engl. 122, 2769-2778 (1996).

Sasai, Y. Cytosystems dynamics in self-organization of tissue architecture. Nature 493, 318-326 (2013).

Shao¹, Y. et al. Self-organized amniogenesis by human pluripotent stem cells in a biomimetic implantation-like niche. Nat. Mater. 16, 419-425 (2017).

Shao², Y. et al. A pluripotent stem cell-based model for post-implantation human amniotic sac development. Nat. Commun. 8, 208 (2017).

Sozen, B. et al. Self-assembly of embryonic and two extra-embryonic stem cell types into gastrulating embryo-like structures. Nat. Cell Biol. 20, 979 (2018).

Tam, P. P. L. & Behringer, R. R. Mouse gastrulation: the formation of a mammalian body plan. Mech. Dev. 68, 3-25 (1997).

Turner, D. A., et al., Wnt/beta-catenin and FGF signalling direct the specification and maintenance of a neuromesodermal axial progenitor in ensembles of mouse embryonic stem cells. Development, 2014. 141(22): p. 4243-53.

Turner, D., et al., Gastruloids develop the three body axes in the absence of extraembryonic tissues and spatially localised signalling. bioRxiv, 2017¹. doi.org/10.1101/104539.

Turner, D., et al., Anteroposterior polarity and elongation in the absence of extra-embryonic tissues and of spatially localised signalling in gastruloids: mammalian embryonic organoids. Development, 2017². 144(21): p. 3894-3906.

Tyser, R. C. et al. Calcium handling precedes cardiac differentiation to initiate the first heartbeat. eLife 5, (2016).

van den Brink, S. C. et al. Symmetry breaking, germ layer specification and axial organisation in aggregates of mouse embryonic stem cells. Development 141, 4231-4242 (2014).

1. A polarised three-dimensional cellular aggregate generated in vitro from one or more pluripotent stem cells, wherein:
(a) the polarised three-dimensional cellular aggregate comprises
i. cells comprising one or more markers characteristic of endodermal cells or derivatives thereof,
ii. cells comprising one or more markers characteristic of mesodermal cells or derivatives thereof, and
iii. cells comprising one or more markers characteristic of ectodermal cells or derivatives thereof; and
(b) the polarised three-dimensional cellular aggregate is polarised along the anterior-posterior, dorsal-ventral and medio-lateral axes, and wherein
i. the anterior-posterior axis is defined by at least an anterior region of cells and a posterior region of cells, wherein the cells of the anterior region express a higher or lower level of one or more genes than the cells of the posterior region,
ii. the dorsal-ventral axis is defined by at least a dorsal region of cells and a ventral region of cells, wherein the cells of the dorsal region express a higher or lower level of one or more genes than the cells of the ventral region, and
iii. the medio-lateral axis is defined by at least a medial region of cells and two lateral regions of cells, wherein the cells of the medial region express a higher or lower level of one or more genes than the cells of the lateral regions.

2. The polarised three-dimensional cellular aggregate of claim 1, wherein the cells of the anterior region express a lower level of one or more genes than the cells of the posterior region, and wherein the one or more genes are selected from Bra, Cdx2, Wnt3a, Cyp26a1, Fgf8, Wnt5a Tbx6, Msgn, Hes3, Chrd, Greb1, Rspo, Notum, Sall3, Spy, Sp8 and Fgf4.

3. The polarised three-dimensional cellular aggregate of claim 1 or claim 2, wherein the cells of the anterior region express a higher level of one or more genes than the cells of the posterior region, and wherein the one or more genes are selected from Gata6, Raldh2, Pax3, Tbx1, Uncx4.1, Pax1, Six1, Meis1, Crabp1, Foxc2, Eya1 and Lmo4.

4. The polarised three-dimensional cellular aggregate of any one of claims 1-3, wherein the anterior-posterior axis is further defined by a central region of cells between the anterior region of cells and the posterior region of cells, wherein the cells of the central region express a higher or lower level of one or more genes than the cells of the anterior or posterior regions.

5. The polarised three-dimensional cellular aggregate of claim 4, wherein the cells of the central region express a higher level of one or more genes than the cells of the anterior or posterior regions, and wherein the one or more genes are selected from Cer1, Jag2, Lefty1, Utf1, Tbx3, Ripply2, Mesp1, and Mesp2.

6. The polarised three-dimensional cellular aggregate of any one of claims 1-5, wherein the polarised three-dimensional cellular aggregate exhibits spatial collinearity of Hox gene expression along the anterior-posterior axis.

7. The polarised three-dimensional cellular aggregate of any one of claims 1-6, wherein the cells of the dorsal region express a lower level of one or more genes than the cells of the ventral region, and wherein the one or more genes are selected from Shh, Krt18, Pgg, Nedd9, Noda1, Lefty1, 2, Tbx6, Msgn, and Kdr.

8. The polarised three-dimensional cellular aggregate of any one of claims 1-7, wherein the cells of the dorsal region express a higher level of one or more genes than the cells of the ventral region, and wherein the one or more genes are selected from Sox2, Lnfg, Irx3, Sox1, and Pax7.

9. The polarised three-dimensional cellular aggregate of any one of claims 1-8, wherein the cells of the medial region express a lower level of one or more genes than the cells of the lateral regions, and wherein the one or more genes are selected from Osr1, Pecam, Meox1, Pax2, Lefty1, and Pitx2.

10. The polarised three-dimensional cellular aggregate of any one of claims 1-9, wherein the cells of the medial region express a higher level of one or more genes than the cells of the lateral regions, and wherein the one or more genes are selected from Sox2, Lfng, FoxA2, and Noto1.

11. The polarised three-dimensional cellular aggregate of any one of claims 1-10, wherein the one or more markers characteristic of endodermal cells are one or more genes the expression of which is characteristic of endodermal cells.

12. The polarised three-dimensional cellular aggregate of claim 11, wherein the one or more genes the expression of which is characteristic of endodermal cells are selected from Gsc, Cdx2, Nedd9, Pyy, Shh, Sores, Cer1, Sox17 and FoxA1.

13. The polarised three-dimensional cellular aggregate of any one of claims 1-12, wherein the one or more markers characteristic of mesodermal cells are one or more genes the expression of which is characteristic of mesodermal cells.

14. The polarised three-dimensional cellular aggregate of claim 13, wherein the one or more markers characteristic of mesodermal cells are selected from, Bra, Meox1, Msgn, Osr1, Pax2, Pecam, Raldh2, Ripply1/2, Tbx6, Tcf15, Uncx4.1, Kdr, and Pecam.

15. The polarised three-dimensional cellular aggregate of any one of claims 1-14, wherein the one or more markers characteristic of ectodermal cells are one or more genes the expression of which is characteristic of ectodermal cells.

16. The polarised three-dimensional cellular aggregate of claim 15, wherein the one or more markers characteristic of ectodermal cells are one or more markers characteristic of neural cells.

17. The polarised three-dimensional cellular aggregate of claim 16, wherein the one or more markers characteristic of neural cells are one or more genes the expression of which is characteristic of neural cells, optionally wherein the one or more genes are selected from Dll1, HesS, Lnfg, Olig2, Pax3, Pax7, Sox1, Sox2, Irx3, Mnx1, Phox2a, Evx2, Ascl1, Id2, and Lhx9.

18. The polarised three-dimensional cellular aggregate of any one of claims 1-17, wherein the polarised three-dimensional cellular aggregate comprises primordial germ cell-like cells (PGCs), optionally wherein the PGCs express Blimp1 and/or AP2g.

19. The polarised three-dimensional cellular aggregate of any one of claims 1-18, wherein the three-dimensional cellular aggregate comprises at least 50 cells, at least 100 cells, at least 200 cells, at least 300 cells, at least 400 cells, at least 500 cells, at least 600 cells, at least 800 cells, at least 900 cells, at least 1000 cells, at least 1500 cells, at least 2000, at least 2500 cells, at least 5000 cells, at least 10,000 cells, at least 15,000 cells, at least 20,000 cells, at least 30,000 cells, at least 40,000 cells or at least 50,000 cells.

20. The polarised three-dimensional cellular aggregate of any one of claims 1-19, wherein the polarised three-dimensional cellular aggregate has a length of at least 0.05 mm, at least 0.1 mm, at least 0.2 mm, 0.3 mm, at least 0.4 mm, at least 0.5 mm, at least 0.6 mm, at least 0.7 mm, at least 0.8 mm, at least 0.9 mm, at least 1 mm or at least 1.5 mm.

21. The polarised three-dimensional cellular aggregate of any one of claims 1-20, wherein the polarised three-dimensional cellular aggregate is elongate from anterior to posterior.

22. The polarised three-dimensional cellular aggregate of any one of claims 1-21, wherein the three-dimensional cellular aggregate comprises one or more progenitor cells or derivatives thereof.

23. The polarised three-dimensional cellular aggregate of any one of claims 1-21, wherein the three-dimensional cellular aggregate comprises one or more haematopoietic progenitors or derivatives thereof.

24. The polarised three-dimensional cellular aggregate of claim 22, wherein the haematopoietic progenitors or derivatives thereof express one or more of Flk1, Scl, Runx1, Gata2, Cxcr4, cKit and CD41.

25. The polarised three-dimensional cellular aggregate of any one of claims 1-21, wherein the three-dimensional cellular aggregate comprises one or more progenitors of the vascular system or derivatives thereof.

26. The polarised three-dimensional cellular aggregate of claim 25, wherein the progenitors of the vascular system express one or more of Flk1, Scl, Runx1, Gata2, Cxcr4, cKit and CD41.

27. The polarised three-dimensional cellular aggregate of any one of claims 1-26, wherein the polarised three-dimensional cellular aggregate comprises one or more cardiac progenitor cells or derivatives thereof.

28. The polarised three-dimensional cellular aggregate of claim 27, wherein the cardiac progenitor cells express one or more cardiac specific genes.

29. The polarised three-dimensional cellular aggregate of claim 28, wherein the one or more cardiac specific genes are selected from Flk1, cTnT, Mic2a CD31, Mesp1, Nkx2-5, Tbx5 and Pitx2.

30. The polarised three-dimensional cellular aggregate of any one of claims 1-29, wherein the polarised three-dimensional cellular aggregate is generated in vitro from one or more embryonic stem cells (ESCs).

31. The polarised three-dimensional cellular aggregate of any one of claims 1-29, wherein the polarised three-dimensional cellular aggregate is generated in vitro from one or more induced pluripotent stem cells (iPSCs).

32. The polarised three-dimensional cellular aggregate of any one of claims 1-29, wherein the three-dimensional cellular aggregate is generated in vitro from one or more epiblast stem cells (EpiSCs).

33. The polarised three-dimensional cellular aggregate of any one of claims 1-32, wherein the three-dimensional cellular aggregate is generated in vitro from a single pluripotent stem cell.

34. A method for obtaining a polarised three-dimensional cellular aggregate, the method comprising:
(a) obtaining a cell suspension, wherein the cell suspension comprises one or more disassociated pluripotent stem cells;
(b) culturing the cell suspension under conditions that promote the transformation of at least one of the disassociated pluripotent stem cells into a three-dimensional cellular aggregate; and
(c) culturing the three-dimensional cellular aggregate under conditions that promote the transformation of the three-dimensional cellular aggregate into a polarised three-dimensional cellular aggregate; and
wherein the polarised three-dimensional cellular aggregate is a polarised three-dimensional cellular aggregate as defined in any one of claims 1-33.

35. A method for obtaining a polarised three-dimensional cellular aggregate, the method comprising:
(a) obtaining a cell suspension, wherein the cell suspension comprises one or more disassociated pluripotent stem cells;
(b) culturing the cell suspension under conditions that promote the transformation of at least one of the disassociated pluripotent stem cells into a three-dimensional cellular aggregate; and
(c) culturing the three-dimensional cellular aggregate under conditions that promote the transformation of the three-dimensional cellular aggregate into a polarised three-dimensional cellular aggregate; and
(d) culturing the polarised three-dimensional cellular aggregate under conditions that promote the differentiation of one or more cells of the polarised-three dimensional cellular aggregate, wherein the conditions comprise shaking the polarised three-dimensional cellular aggregate.

36. A method for obtaining a polarised three-dimensional cellular aggregate, the method comprising:
(a) obtaining a cell suspension, wherein the cell suspension comprises one or more disassociated pluripotent stem cells;
(b) culturing the cell suspension under conditions that promote the transformation of at least one of the disassociated pluripotent stem cells into a three-dimensional cellular aggregate;
(c) culturing the three-dimensional cellular aggregate under conditions that promote the transformation of the three-dimensional cellular aggregate into a polarised three-dimensional cellular aggregate; and
(d) culturing the polarised three-dimensional cellular aggregate under conditions that promote the differentiation of one or more cells of the polarised-three dimensional cellular aggregate into progenitor cells or derivatives thereof.

37. A method for obtaining a polarised three-dimensional cellular aggregate, the method comprising:
(a) obtaining a cell suspension, wherein the cell suspension comprises one or more disassociated pluripotent stem cells;
(b) culturing the cell suspension under conditions that promote the transformation of at p least one of the disassociated pluripotent stem cells into a three-dimensional cellular aggregate;
(c) culturing the three-dimensional cellular aggregate under conditions that promote the transformation of the three-dimensional cellular aggregate into a polarised three-dimensional cellular aggregate; and
(d) culturing the polarised three-dimensional cellular aggregate under conditions that promote the emergence of spatially organized regions comprising differentiated cells and progenitor cells within the polarised-three dimensional cellular aggregate.

38. The method of claim 36 or 37, wherein the one or more progenitor cells or derivatives thereof are:
a. haematopoietic progenitor cells and/or derivatives thereof;
b. cardiac progenitor cells and/or derivatives thereof;
c. paraxial mesoderm and/or derivatives thereof;
d. somites and/or derivatives thereof;
e. neural crest and/or derivatives thereof;
f. neural ectoderm and/or derivatives thereof;
g. placodal ectoderm and/or derivatives thereof;
h. intermediate mesoderm progenitor cells and/or derivatives thereof;
i. axial mesoderm progenitor cells;
j. neuromesodermal progenitor cells and/or derivatives thereof;
k. lateral plate mesoderm and/or derivatives thereof;
l. primordial germ cells and/or derivatives thereof;
m. node cells and/or derivatives thereof; and/or n. endoderm and/or derivatives thereof.

39. The method of any one of claims 34-38, wherein the step of culturing the polarised three-dimensional cellular aggregate comprises culturing the polarised three-dimensional cellular aggregate in a medium comprising FGF2 and VEGF.

40. The method of claim 39, wherein the progenitor cells or derivatives thereof are haematopoietic progenitor cells or derivatives thereof, or cardiac progenitor cells or derivatives thereof.

41. The method of any one of claims 34-40, wherein the step of culturing the three-dimensional cellular aggregate comprises culturing the three-dimensional cellular aggregate in a medium comprising an activator of Wnt signalling.

42. The method of any one of claims 34-41, wherein the one or more disassociated pluripotent stem cells are one or more embryonic stem cells (ESCs).

43. The method of any one of claims 34-41, wherein the one or more disassociated pluripotent stem cells are one or more induced pluripotent stem cells (iPSCs).

44. The method of any one of claims 34-43, wherein the one or more disassociated pluripotent stem cells is a single pluripotent stem cell.

45. The method of any one of claims 34-44, wherein prior to the step of disassociating the pluripotent stem cells, the method further comprises the step of culturing the pluripotent stem cells in a medium comprising one or more of an inhibitor of ERK signalling, an inhibitor of FGF signalling, an inhibitor of MEK signalling, an activator of Wnt signalling and/or Leukaemia Inhibitory Factor.

46. The method of any one of claims 34-45, wherein step (b) comprises culturing the cell suspension for 24-72 hours, 30-66 hours, 36-60 hours, 42-54 hours, 44-52 hours, 46-50 hours, 47-49 hours or 48 hours.

47. The method of any one of claims 34-46, wherein step (c) comprises culturing the three-dimensional cellular aggregate for 1-48 hours, 6-42 hours, 12-36 hours, 18-30 hours, 20-28 hours, 22-26 hours, 23-25 hours or 24 hours.

48. The method of any one of claims 34-47, wherein step (d) comprises culturing the polarised three-dimensional cellular aggregate for 24-120 hours, 30-96 hours, 36-72 hours, 42-54 hours, 44-52 hours, 46-50 hours, 47-49 hours or 48 hours.

49. The method of any one of claims 34-48, wherein one or more of steps (b)-(d) further comprise shaking the three-dimensional cellular aggregate or polarised three-dimensional cellular aggregate.

50. The method of any one of claims 34-49, wherein the cell suspension, three-dimensional cellular aggregate and polarised three-dimensional cellular aggregate are cultured for a total of at least 120 hours, at least 130 hours, at least 140 hours, at least 150 hours, at least 160 hours, at least 170 hours, at least 180 hours, at least 190 hours, at least 200 hours, at least 210 hours, at least 220 hours, at least 230 hours, at least 240 hours or at least 250 hours.

51. A method for obtaining a polarised three-dimensional cellular aggregate, the method comprising:
(a) culturing one or more epiblast pluripotent stem cells, wherein the step of culturing comprises
a. culturing the epiblast pluripotent stem cells in a medium comprising FGF and Activin,
b. culturing the epiblast pluripotent stem cells in a medium comprising FGF, and
c. culturing the epiblast pluripotent stem cells in a medium comprising FGF and an activator of Wnt signalling;
(b) generating a cell suspension from the cultured epiblast pluripotent stem cells, wherein the cell suspension comprises one or more disassociated epiblast pluripotent stem cells; and
(c) culturing the cell suspension under conditions that promote the transformation of at least one of the disassociated epiblast pluripotent stem cells into a polarised three-dimensional cellular aggregate.

52. The method of claim 51, wherein step (c) comprises culturing the cell suspension in a medium comprising an activator of Wnt signalling.

53. The method of claim 51, wherein step (c) comprises culturing the cell suspension in a medium comprising an activator of Wnt signalling and FGF.

54. A method for obtaining one or more progenitor cells or derivatives thereof, the method comprising
(a) obtaining a polarised three-dimensional cellular aggregate according to the method of any one of claims 34-53; and
(b) isolating from the polarised three-dimensional cellular aggregate one or more progenitor cells or derivatives thereof.

55. A polarised three-dimensional cellular aggregate obtainable by the method of any one of claims 34-53.

56. A progenitor cell or derivative thereof obtainable by the method of claim 54.
